(19) United States
(12) Patent Application Publication
(10) Pub. No.: US 2014/0161720 A1
(43) Pub. Date: Jun. 12, 2014

(54) TREATMENT OF ANGIOGENIC- OR VASCULAR-ASSOCIATED DISEASES

(71) Applicants: Igor Garkavtsev, Cambridge, MA (US); Rakesh K. Jain, Wellesley, MA (US)

(72) Inventors: Igor Garkavtsev, Cambridge, MA (US); Rakesh K. Jain, Wellesley, MA (US)

(73) Assignee: THE GENERAL HOSPITAL CORPORATION, Boston, MA (US)

(21) Appl. No.: 14/119,532

(22) PCT Filed: May 25, 2012
(86) PCT No.: PCT/US2012/039622

§ 371 Date: Feb. 3, 2014

Related U.S. Application Data
(60) Provisional application No. 61/490,525, filed on May 26, 2011.
(60) Provisional application No. 61/497,813, filed on Jun. 16, 2011.

(51) Int. Cl. C07D 311/92; A61K 36/185; A61K 31/352; A61K 45/06; A61K 47/30

(52) U.S. Cl. CPC C07D 311/92; A61K 45/06; A61K 47/30; A61K 31/352; A61K 36/185
USPC 424/1.11; 424/450; 514/454; 514/455

(57) ABSTRACT

Described herein are methods and compositions comprising a compound of formula (I), e.g., dehydro-alpha-lapachone, or an analog, derivative, isomer, prodrug, or pharmaceutically acceptable salt thereof, for treatment and/or prevention of angiogenic- or vascular-associated diseases or disorders. The compound has anti-vascular activity. In some embodiments, the compound has anti-vascular activity that targets pathways other than VEGF pathways. In some embodiments, the compound or the composition further comprises anti-tumor activity. In some embodiments, the compound or the composition can decrease adhesion or motility of at least one cell (e.g., endothelial cells or cancer cells).

### CROSS-REFERENCE TO RELATED APPLICATIONS

This application claims benefit under 35 U.S.C. §119(e) of the U.S. Provisional Application Nos. 61/490,525 filed May 26, 2011, and 61/497,813 filed Jun. 16, 2011, the contents of which are incorporated herein by reference in their entirety.

### GOVERNMENT SUPPORT

This invention was made with federal funding under Grant No. W81XWH-10-1-0016, awarded by the Department of Defense, and Grant No.: PO1 CA080124 awarded by National Institutes of Health. The U.S. government has certain rights in the invention.

### TECHNICAL FIELD

Provide herein relates generally to compositions and methods for the treatment of diseases or disorders involving abnormal vasculatures or angiogenesis.

### BACKGROUND OF THE DISCLOSURE

Blood vessels are the means by which oxygen and nutrients are supplied to living tissues and waste products are removed from living tissue. Angiogenesis refers to a process by which new blood vessels are formed. It is essential in reproduction, development and wound repair. However, abnormal or excessive angiogenesis can have adverse consequences. For example, solid tumors are vascularized as a result of angiogenesis, enabling them to obtain oxygen and nutrients that permit them to grow rapidly and metastasize. Because maintaining the rate of angiogenesis in its proper equilibrium is so critical to a range of functions, it must be carefully regulated in order to maintain health. The angiogenesis process is believed to begin with the degradation of the basement membrane by proteases secreted from endothelial cells (EC) activated by mitogens such as vascular endothelial growth factor (VEGF) and basic fibroblast growth factor (bFGF). The cells migrate and proliferate, leading to formation of endothelial cell sprouts into a stromal space, then, vascular loops are formed and capillary tubes develop with formation of tight junctions and deposition of new basement membrane.

The rate of angiogenesis involves a change in the local equilibrium between positive and negative regulators of the growth of microvessels. In adults, the proliferation rate of endothelial cells is typically low compared to other cell types in the body. Physiological exceptions in which angiogenesis results in rapid proliferation typically occur under tight regulation, such as in the female reproduction system and during wound healing.

However, unregulated angiogenesis resulting in abnormal vasculatures or neovascularization can cause various different diseases. For example, in arthritis, new capillary blood vessels invade the joint and destroy cartilage. In diabetes, new capillaries invade the vitreous, bleed, and cause blindness. Ocular neovascularization is the most common cause of blindness. Tumor growth and metastasis are angiogenesis-dependent.

Current treatments of these angiogenesis-dependent diseases are inadequate. While antiangiogenic agents, most of which target VEGF or its receptors, are emerging as standard therapies for several major human cancers (3-5), antiangiogenic therapy unfortunately leads to modest efficacy, inherent or acquired resistance, and rare but life-threatening toxicity (6-7). In addition, patients treated with some antiangiogenic agents, e.g., AVASTIN®, can acquire resistance to the anti-angiogenic agent over time. As such, there is a strong need for development of better antivascular agents that target pathways other than VEGF, and are potent with relatively low or no toxicity.

### SUMMARY

Embodiments provided herein are based on, at least in part, the discovery of an anti-vascular agent derived from Tabebuia Avellaneda tree, e.g., for use in treatment of conditions involving abnormal vasculatures or angiogenesis. For example, the inventors have demonstrated that dehydro-α-lapachone (DAL), one of the natural products derived from Tabebuia Avellaneda tree, inhibited normal vascular development or neovascularization in an in vivo zebrafish model. Further, the inventors have demonstrated that DAL reduced vascular density and thus tumor growth in an in vivo mouse model. In addition, the inventors have demonstrated low toxicity of DAL, e.g., up to at least about 100 mg/kg, in an in vivo mouse model. Thus, the findings provide methods and compositions for treating a condition involving abnormal vasculatures or angiogenesis.

Accordingly, provided herein is a method of treating or preventing a condition involving abnormal vasculatures, including, but not limited to, tuberculosis, autoimmune diseases, wound repair, hypertrophic scar or keloid, neointimal hyperplasia, psoriasis, macular degeneration, age-related macular degeneration (AMD), thyroid hyperplasia, preeclampsia, hemangiomas, rheumatoid arthritis and osteoarthritis, Alzheimer's disease, obesity, pleural effusion, atherosclerosis, endometriosis, diabetic retinopathies, other retinopathies, ocular neovascularizations, edemas, chronic obstructive pulmonary diseases, asthma, cystic fibrosis, transplant rejection, allergic reaction, multiple sclerosis, epithelial infection, and conditions involving or characterized by vascular hyperpermeability, inflammation, angiogenesis or vascular hyperproliferation. In some embodiments, the methods provided herein can be used to treat or prevent cancer. In some embodiments, the methods provided herein can be used to treat or prevent metastatic tumors (e.g., a metastatic breast tumor). In one embodiment, the methods provided herein can be used to treat or prevent triple-negative breast tumor (e.g., estrogen receptor-negative, progesterone receptor-negative and C-erbB-2-negative; ER-PR-HER2-).

In another aspect, provided herein is a method of treating or preventing a condition involving abnormal vasculatures, wherein the condition excludes cancer or metastatic tumors.

In a further aspect, provided herein relates to a method of treating or preventing an ocular disease or disorder involving abnormal vasculatures. Exemplary ocular diseases or disorders involving abnormal vasculatures include, but are not limited to, age-related macular degeneration (AMD), proliferative diabetic retinopathy (PDR), diabetic macular edema (DME), neovascular glaucoma, corneal neovascularization (trachoma), and pterygium.

In any aspects of any embodiment of the methods described herein, the method includes administering to a subject a composition comprising a compound of formula (I) or an analog, derivative, isomer, prodrug, or pharmaceutically acceptable salt thereof, wherein the compound of formula (I) has anti-vascular activity with a structure:

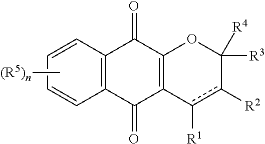

wherein:
R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R³ and R⁴ are each independently selected from hydrogen, alkyl, alkenyl, cyclyl, heterocyclyl, aryl, heteroaryl, each of which can be optionally substituted;
R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R⁶ is independently selected for each occurrence, hydrogen, alkyl, alkenyl, alkynyl, cyclyl, heterocyclyl, aryl, or heteroaryl, each of which can be optionally substituted;
the dotted line between the carbons to which R¹ and R² are bonded represents an optional double bond; and
n is 0, 1, 2, 3, or 4.

Some embodiments described herein provide a method of inhibiting or reducing angiogenesis in a subject in need thereof, the method comprising administering to the subject a composition comprising an anti-vascular compound described herein. In some embodiments of any methods described herein, the subject in need thereof can be diagnosed with or at risk of having an angiogenic-associated disease or a condition involving abnormal vasculatures described herein. In some embodiments, the subject in need thereof can have undergone treatment or is now receiving treatment. In some embodiments, the subject in need thereof can have the disease or disorder described herein in remission.

In some embodiments, the inventors have demonstrated that DAL can interfere with adhesive properties of endothelial cells and/or in vitro vascular network formation. Accordingly, a method of decreasing adhesion or motility of at least one cell (e.g., endothelial cell or cancer cell) is also provided herein. Such method comprising administering a composition comprising an anti-vascular compound described herein.

In some embodiments, the inventors have demonstrated that DAL can inhibit or reduce activity of an immunomodulatory enzyme (e.g., indoleamine 2,3-dioxygenase (IDO)). Accordingly, one aspect provided herein relates to an immunomodulatory composition comprising an effective amount of a compound of formula (I) described herein, or an analog, derivative, isomer, prodrug, or pharmaceutically acceptable salt thereof. In some embodiments, the compound included in the immunomodulatory composition can inhibit or reduce activity of an immunomodulatory enzyme (e.g., but not limited to, indoleamine 2,3-dioxygenase (IDO)), e.g., by at least about 10%, or more. In some embodiments, the compound included in the immunomodulatory composition can be DAL or an analog, derivative, isomer, prodrug, or pharmaceutically acceptable salts thereof.

The immunomodulatory compositions described herein can increase activity of an immune system in a subject. Thus, methods for increasing activity of an immune system in a subject comprising administering to a subject in need thereof the compound or immunomodulatory composition described herein are also provided herein. In some embodiments where the subject is diagnosed with or at risk of having cancer or metastatic tumor, increasing the activity of the immune system in the subject can produce an anti-tumor effect.

In some embodiments of any aspects of the methods described herein, the composition (including anti-vascular composition and/or immunomodulatory composition) can be administered in a combination therapy. In some embodiments, the combination therapy can comprise administering to the subject at least one active agent. Examples of such active agent include, but are not limited to, therapeutic agents, anti-angiogenic or anti-vascular agents, anti-inflammation agents, VEGF inhibitors, antibiotics, anti-viral agents, anti-fugal agents, anti-protozoal agents, hormones, radioactive agents, toxins, and any combinations thereof.

In some embodiments of the methods described herein, e.g., for treatment of cancer or metastatic tumors, the composition described herein can be administered in combination with at least one anti-cancer agent.

In some embodiments of the methods described herein, e.g., for treatment of ocular diseases or disorders involving abnormal vasculatures, the composition described herein can be administered in combination with an anti-angiogenic therapy, e.g., for ophthalmic applications. Non-limiting examples of anti-angiogenic therapy that can be used for ophthalmic applications can include an anti-VEGF aptamer (e.g., pegaptanib, MACUGEN®); a Fab fragment of a monoclonal antibody directed against VEGF-A (ranibizumab, LUCENTIS® or bevacizumab, AVASTIN®); or a combination thereof.

In various embodiments of any aspects of the methods described herein, the composition can be administered in pulses or by sustained release.

Further provided herein is an anti-vascular composition comprising an anti-vascular compound of formula (I) described herein or an analog, derivative, isomer, prodrug, or pharmaceutically acceptable salt thereof, wherein the anti-vascular compound is adapted for a controlled release for at least 2 months in a subject in need thereof.

In some embodiments, the anti-vascular composition adapted for a controlled release can be encapsulated in one or more polymeric vehicles. Non-limiting examples of polymeric vehicles include polylactic and/or polyglycolic acids, poly(lactic-co-glycolic acid), polyethylene glycol, polyanhydrides, poly(sebacic acid-co-ricinoleic acid), polycaprolactones, copolyoxalates, polyesteramides, polyorthoesters, polyhydroxybutyric acid, and any combinations thereof.

In some embodiments, the anti-vascular composition can further comprise at least one active agent, for example, selected from the group consisting of therapeutic agents, anti-angiogenic or anti-vascular agents, anti-inflammation agents, VEGF inhibitors, antibiotics, anti-viral agents, anti-fugal agents, anti-protozoal agents, hormones, radioactive agents, toxins, and any combinations thereof. In some embodiments, the anti-vascular composition can further comprise at least one anti-cancer agent or anti-neoplastic agent.

For some embodiments of the methods and/or compositions provided herein, the compound described herein can have anti-tumor activity, and/or inhibit or decrease immune tolerance to the condition being treated.

In some embodiments of any aspects provided herein, the compound described herein can be a product from Tabebuia Avellaneda tree. In one embodiment, such compound is dehydro-alpha-lapachone or an analog, derivative, isomer, prodrug, or pharmaceutically acceptable salt thereof. In various embodiments, the compound can be further modified, e.g., conjugated to a macromolecule.

In some embodiments of the methods and/or compositions described herein, an effective amount of the composition or the compound of formula (I) administered to a subject can vary from about 0.001 mg/kg to about 500 mg/kg, from about 1 mg/kg to about 100 mg/kg, or from about 10 mg/kg to about 50 mg/kg. In one embodiment, the effective amount of the composition or the compound described herein is from about 10 mg/kg to about 50 mg/kg.

In one embodiment, the method provided herein, for treating or preventing a condition involving abnormal vasculatures, includes administering to a subject an anti-vascular composition comprising dehydro-alpha-lapachone or an analog, derivative, isomer, prodrug, or pharmaceutically acceptable salt thereof. In such embodiment, the anti-vascular composition can further comprise at least one active agent.

### BRIEF DESCRIPTION OF THE DRAWINGS

FIGS. 1A to 1E show a schematics of an exemplary drug screen for antivascular agent described herein, e.g., dehydro-alpha-lapachone (DAL) and effects of DAL on cell adhesion properties. FIG. 1A shows a schematic of an exemplary high-throughput drug screen. Of the 50,000 compounds screened, 86 compounds were selected to test in the secondary assays. Only compounds that made MDA-MB-231 cells more rounded, with less spreading than control cells, and interfered with actin regulation were selected for further study. All these selected small molecules were further passed through a number of filtering toxicity assays to eliminate toxic compounds. To confirm the absence of toxicity in the cellular model for each candidate molecule, we repeated the experiments and re-assayed at three different concentrations (1.67 μM, 5 μM and 10 μM). The non-toxic compounds were then analyzed in a zebrafish model. Small molecules that did not alter zebrafish survival during embryonic development and adult life for further experiments were used. Finally, out of 50,000 compounds, two non-toxic compounds that demonstrated anti-vascular properties were selected. One of these compounds, DAL was selected for further experiments. FIG. 1B shows the structure of DAL, a natural product. FIG. 1C shows that DAL-treated cells lost their adhesion properties. FIG. 1D shows that untreated MDA-MB-231 control cells spread more rapidly on fibronectin-coated plates than cells treated with DAL. In contrast, cells treated with DAL were significantly smaller than control cells and had a rounded shape. FIG. 1E shows that DAL is involved in actin cytoskeleton assembly regulation. For F-actin visualization, MDA-MB-231 cells were plated on fibronectin and treated with each selected compound (5 μM) for two hours. After fixing and permeabilizing these cultured cells, they were subsequently stained with phalloidin. The untreated control cells spread more rapidly on fibronectin-coated plates than those treated with DAL. In contrast, cells treated with DAL were significantly smaller than control cells and had a rounded shape. In the control cells, actin stress fibers were clearly visible and aligned with the long axis of the cells. Meanwhile, in cells treated with DAL, actin was accumulated close to the membrane. Cytochalasin D was used as positive control that inhibits actin assembly.

FIGS. 2A to 2C show that DAL impairs vessel development and regeneration in zebrafish. FIG. 2A shows a macroscopic image of 48 hours after fertilization Tg(fli1:EGFP)^{y1} zebrafish embryos, comparing control embryos and embryos treated with DAL (5 μM). Treated embryos failed to form vessel branches. FIGS. 2B and 2C are results from regeneration experiments with zebrafish, depicting the vasculature of the caudal fin on 2 days and 9 days after amputation in control and treated zebrafish. White lines indicate the amputation plane. FIG. 2B shows vascular plexus formation, plexus remodeling, and late regenerative angiogenesis in the caudal fins of both wild-type control (fli1:EGFP) and DAL-treated fish 2 days (top) and 9 days (bottom) after amputation. FIG. 2C is an image with a higher magnification of the reconnected vessels. Nine days after amputation, the regenerating vasculature of each fin ray (three fin rays are shown) consists of a plexus with dense unstructured vessels extending distally from the amputation plane (white arrowheads). Regenerating blood vessels in the wild-type control (fli1:EGFP) form plexuses. Fish treated with DAL have defects in anastomosis and plexus formation. (Scale bars: 200 μm)

FIGS. 3A to 3D show the anti-vascular effect of DAL in mouse models. FIG. 3A shows a set of representative intravital images of tumor vessels in mice bearing orthotopic 4T1 mammary tumors through a treatment course. The tumors were imaged with multiphoton microscopy on day 0, day 2, and day 4; daily treatments started on day 0 after the first images were collected. Images are 3D projections of tissue from 0-200 μm, with pseudocolor labeling by depth. FIG. 3B shows that tumors in mice treated with DAL have lower vascular volume fractions than tumors in saline-treated mice at day 4 (p=0.0017). FIG. 3C shows that total tumor vascular length (normalized to tumor volume) is lower in DAL-treated mice than in saline-treated mice (p=0.0069 day 2, p=0.024 day 4). FIG. 3D shows that there is no significant difference in mean tumor vascular diameter between DAL-treated and saline-treated mice. The results in FIGS. 3C and 3D indicate that vessel pruning as the mechanism for decreased vascular volume fraction. n=5 for the saline group, n=4 for the DAL group.

FIGS. 4A and 4B show antitumor effects of DAL in orthotopic mouse models. FIG. 4A shows tumor growth curves for 4T1 (n=4) and E0771 orthotopic mammary tumors (n=5). Tumor-bearing female SCID mice were treated with daily intraperitoneal injections of saline or about 37.5 mg/kg DAL. Throughout the study, tumor size was measured on alternating days. Since all tumors grew at different rates to the start size, tumors were time- and size-matched for their initial treatment on day 0 just after the first measurement. FIG. 4B shows that DAL increased the doubling time for 4T1 tumors from 2.20+/−0.07 days to 11.21+/−3.53 days (p=0.044) and that for E0771 tumors from 2.65+/−0.27 days to 4.77+/−0.58 days (p=0.010).

FIGS. 5A to 5C show that DAL interferes with adhesive properties of endothelial cells in vitro. FIG. 5A shows that DAL is involved in actin cytoskeleton assembly regulation. For F-actin visualization, HUVECs were plated and treated with saline or DAL (10 μM) for three hours. After fixing and permeabilizing these cultured cells, the cells were subsequently stained with phalloidin. FIG. 5B shows that DAL interferes with adherens junctions in networks of HUVEC cells cultured on matrigel. After 24 hours, control HUVEC cells organized into a network of cordlike structures while DAL-treated HUVECs show disjoined networks. Existing cordlike structures, when exposed to 10 μM of DAL for 3 hours, were also disrupted. FIG. 5C shows results of a wound-healing motility assay with HUVECs. HUVEC monolayers were disrupted mechanically with a pipette tip and the migration of cells to “heal” the wound was monitored using microscopy. DAL (5 μM) slowed healing compared to the control, shown at 24 hours after wounding.

FIGS. 6A to 6C show that DAL decreases Rac1 activity and activates Rac1 degradation. FIG. 6A shows detection of RhoA activity in HUVEC cells treated with DAL. FIG. 6B shows that DAL decreases level of Rac1 in HUVECs. HUVEC cells were treated with 10 μM of DAL for 24 hours and then lysed. A western blot with anti-human Rac1 antibody is shown on top panel while the bottom panel represents a loading control. FIG. 6C shows that treatment with DAL increases Rac1 ubiquitination in HUVEC cells. To enhance ubiquitination events, HUVEC cells were pretreated with the proteasome inhibitors lactacystin (2.5 mM) or MG-132 (0.2 mM) for one hour and subsequently treated with DAL (10 μM) of DAL for 24 hours. Duolink in situ was used for the detection of ubiquitinated Rac1 in DAL treated and untreated HUVEC cells. For the two Duolink PLA probes, primary mouse antibodies raised against human Rac1 and primary rabbit anti-ubiquitin were used. Fluorescent spots represent ubiquitinated Rac1. The nuclei with additionally stained DAPI.

FIGS. 7A and 7B show that DAL regulates IDO1 activity through binding. FIG. 7A shows dose-dependent activity of DAL in HEK 293 cells with overexpression construct for IDO. The IDO activity was analyzed by the presence of kynurenine with a spectrophotometric assay. DAL was tested in a 10-dose IC50 mode with threefold serial dilutions (0.1 μM, 0.3 μM, 0.9 μM, 2.7 μM, 8.1 μM, 24.3 μM, 72.9 μM, 218.7 μM, 656 μM and 2000 μM). FIG. 7B shows the predicted structural mechanism of IDO inhibition by DAL. DAL binds in the allosteric pocket that is occupied by buffer molecules in the existing IDO structures (CHES, N-cyclohexyl-2-aminoethanesulfonic acid). DAL binds concurrently with the tryptophan substrate (Trp) and prevents the closure of the binding site lid that is critical for the reaction catalysis.

FIG. 8 is a graph showing pharmacokinetic parameters after intraperitoneal administration of DAL. The graph shows Average Plasma Concentration (ng/mL) of DAL after intraperitoneal administration (i.p.) in 100% DMSO formulation into male CD-1 mice versus time following i.p. Pharmacokinetic studies for DAL indicate that its half-life is 1.7 hours in plasma after i.p. dosing.

FIGS. 9A-9D show diagrams of comparative modeling and molecular docking of DAL. FIG. 9A shows Indole-2,3-oxygenase (IDO) X-ray structure (PDB 2d0t). The open/disordered loop in the vicinity of the catalytic site. FIG. 9B shows IDO X-ray binding pocket. FIG. 9C shows that DAL was docked in the pocket modeled with a substrate—hence a non-competitive compound-binding mode. FIG. 9D shows superimposition with the closed binding site lid, indicating that the DAL, in some embodiments, can prevent the lid from closing, hence inhibiting the oxidation reaction.

FIG. 10 is a schematic diagram showing the two-phase metastatic cascade. Phase 1: Breast cancer cells move and leave the primary tumor through intravasation. Phase 2: Cancer cells arrive at a secondary site where they need to move, survive and adapt to a new microenvironment in order to form a metastatic lesion. In accordance with some embodiments described herein, dehydro-alpha-lapachone (DAL) can affect cancer cell motility, anti-tumor immunity and/or metastasis development. As shown in Example 8, orthotopically implanted triple-negative breast cancer cells are used. (This figure is adapted and modified from Chaffer and Weinberg (2011) Science 331: 1559-1564)

FIG. 11 shows an exemplary experimental protocol for evaluation of effects of DAL on triple-negative breast cancer. In such protocol, DAL is administered alone and in combination with paclitaxel on lung metastasis using triple-negative breast cancer metastasis mouse models.

### DETAILED DESCRIPTION OF PREFERRED EMBODIMENTS

Described herein are methods and compositions for treating or preventing a condition involving abnormal vasculatures. In some embodiments, the methods and compositions herein can be used to reduce or inhibit angiogenesis or existing blood vessels in a subject in need thereof. In some embodiments, the methods and compositions herein can be used to decrease adhesion and/or motility of endothelial cells or cancer cells. In accordance with various embodiments provided herein, a compound of formula (I) provided herein can serve as an antivascular agent with relatively low toxicity for treatment or prevention of various angiogenic- or vascular-associated diseases or disorders. In one embodiment, the compound of formula (I) is a natural plant product derived from Tabebuia Avellaneda tree, e.g., dehydro-alpha-lapachone (DAL), which shows no signs of toxicity in an mouse model at a concentration up to at least about 100 mg/kg. Not wishing to be bound by theory, the antivascular activity of DAL can be mediated by modulating Rac1 activity and protein levels, and/or destabilizing cell adhesion through the actin cytoskeleton. The inventors have demonstrated the ability of DAL to inhibit normal vascular development or neovascularization (e.g., during wound healing) in an in vivo zebrafish model. Further, the inventors have demonstrated that DAL can reduce vascular density in tumors and thus inhibit tumor growth in an in vivo mouse model. Such discovery of DAL as an anti-vascular agent provides a safer therapeutic agent, whether being administered alone or in conjunction with another therapeutic agent, for treatment or prevention of various diseases or disorders mediated by abnormal vasculatures or angiogenesis.

### Methods of Treatment or Prevention

In one aspect, provided herein is a method of treating or preventing a condition involving abnormal vasculatures, the method comprising administering to a subject in need thereof a composition comprising a compound of formula (I) as disclosed herein or an analog, derivative, isomer, prodrug, or pharmaceutically acceptable salt thereof, wherein the compound of formula (I) has anti-vascular activity.

In some embodiments, the condition involving abnormal vasculatures can include cancer. In some embodiments, the condition involving abnormal vasculatures can include metastatic cancer. Specifically, the inventors have demonstrated, in one embodiment, that DAL can inhibit activity of an immunomodulatory enzyme, e.g., indoleamine 2,3-dioxygenase (IDO). Without wishing to be bound by theory, patients with triple-negative cancers generally have increases in tryptophan catabolism through elevation of the enzyme IDO, which has immunosuppressive characteristics. Accordingly, a further aspect provided herein relates to a method of treating or preventing triple-negative breast cancer in a subject, wherein the method comprises administering to a subject diagnosed with or at risk of having triple-negative breast cancer a composition comprising a compound of formula (I) as disclosed herein or an analog, derivative, isomer, prodrug, or pharmaceutically acceptable salt thereof, wherein the compound of formula (I) has anti-vascular activity. As used herein, the term “triple-negative breast cancer” refers to a breast cancer subtype with estrogen receptor-negative, progesterone-negative, and C-erbB-2-negative. The triple-negative breast cancer is generally the most aggressive breast cancer subtype and has the worst prognosis with no effective therapies available.

In some embodiments, the compound and/or the composition can inhibit metastatic spread (e.g., indicated by presence of at least one metastasis in an organ distant from a site of a primary tumor, e.g., detected by imaging such as X-ray or CT scan) by at least about 10%, at least about 20%, at least about 30%, at least about 40%, at least about 50%, at least about 60%, at least about 70% or more, as compared to without administration of the compound and/or the composition described herein. In some embodiments, the compound and/or the composition can inhibit metastatic spread (e.g., indicated by presence of at least one metastasis in an organ distant from a site of a primary tumor, e.g., detected by imaging such as X-ray or CT scan) by at least about 70%, at least about 80%, at least about 90%, at least about 95% or more, as compared to without administration of the compound and/or the composition described herein. In some embodiments, the compound and/or the composition can completely inhibit metastatic spread (e.g., no formation of metastases in any organ distant from a site of a primary tumor, e.g., detected by imaging such as X-ray or CT scan) for a period of time, as compared to without administration of the compound and/or the composition described herein. Stated another way, the compound and/or the composition can delay metastatic spread (e.g., no formation of metastases in any organ distant from a site of a primary tumor, e.g., detected by imaging such as X-ray or CT scan) by at least about 1 week, at least about 2 weeks, at least about 3 weeks, at least about 1 month, at least about 2 months, at least about 3 months or more, as compared to without administration of the compound and/or the composition described herein. In some embodiments, the compound and/or the composition can delay metastatic spread delay metastatic spread (e.g., no formation of metastases in any organ distant from a site of a primary tumor, e.g., detected by imaging such as X-ray or CT scan) by at least about 3 months, at least about 6 months, at least about 9 months, at least about 1 year, at least about 2 years, at least about 3 years or more, as compared to without administration of the compound and/or the composition described herein.

In some embodiments, the compound and/or the composition can inhibit or reduce primary and/or metastatic tumor growth (e.g., detected by imaging methods such as X-ray, CT scan or MRI) by at least about 10%, at least about 20%, at least about 30%, at least about 40%, at least about 50%, at least about 60%, at least about 70% or more, as compared to without administration of the compound and/or the composition described herein. In some embodiments, the compound and/or the composition can inhibit or reduce primary and/or metastatic tumor growth (e.g., detected by imaging methods such as X-ray, CT scan or MRI) by at least about 70%, at least about 80%, at least about 90%, at least about 95% or more, as compared to without administration of the compound and/or the composition described herein. In some embodiments, the compound and/or the composition can delay primary and/or metastatic tumor growth (e.g., detected by imaging methods such as X-ray, CT scan or MRI) by any period of time, e.g., by at least about 1 week, at least about 2 weeks, at least about 3 weeks, at least about 1 month, at least about 2 months, at least about 3 months, at least about 6 months, at least about 9 months, at least about 12 months, at least about 2 years, at least about 3 years or longer, as compared to without administration of the compound and/or the composition described herein.

Another aspect provided herein is a method of treating or preventing a condition involving abnormal vasculatures in a subject, wherein the condition excludes any cancer or metastatic tumors. In such aspect, the method comprises administering to a subject in need thereof a composition comprising a compound of formula (I) as disclosed herein or an analog, derivative, isomer, prodrug, or pharmaceutically acceptable salt thereof, wherein the compound of formula (I) has anti-vascular activity.

In some embodiments, the methods provided herein can be used to treat or prevent a condition involving abnormal vasculatures in an eye of a subject. Accordingly, provided herein is also a method to treat or prevent an ocular disease or disorder involving abnormal vasculatures in a subject. Such method comprises administering to a subject diagnosed with or at risk of having an ocular disease or disorder involving abnormal vasculatures a composition comprising a compound of formula (I) as disclosed herein or an analog, derivative, isomer, prodrug, or pharmaceutically acceptable salt thereof, wherein the compound of formula (I) has anti-vascular activity. In some embodiments, the composition is administered directly to the subject's eye diagnosed with or at risk of having an ocular disease or disorder involving abnormal vasculatures.

Exemplary ocular disease or disorders that are amenable to some embodiments of the method described herein include, but are not limited to, age-related macular degeneration (AMD), proliferative diabetic retinopathy (PDR), diabetic macular edema (DME), neovascular glaucoma, corneal neovascularization (trachoma), and pterygium.

In some embodiments, the compound of formula (I) can be administered in combination with an active agent described herein. In some embodiments, the active agent can include an antiangiogenic therapy, e.g., for an ophthalmic application. Examples of an antiangiogenic therapy for ophthalmic applications can include, without limitations, an anti-VEGF aptamer (pegaptanib, MACUGEN®); a Fab fragment of a monoclonal antibody directed against VEGF-A (ranibizumab, LUCENTIS® or bevacizumab, AVASTIN®); or a combination thereof.

In accordance with some embodiments described herein, the methods and/or compositions can be used to inhibit or reduce angiogenesis in a subject in need thereof, e.g., a subject diagnosed with or at risk of having a disease or a condition involving abnormal vasculatures described herein. In some embodiments, the methods and/or compositions can be used to prune or reduce existing blood vessels (e.g., in a target tissue) in a subject in need thereof, e.g., a subject diagnosed with or at risk of having a disease or a condition involving abnormal vasculatures described herein.

The inventors have also demonstrated, in one or more embodiments, that DAL can reduce adhesive and/or motility properties of a cell. In some embodiments, DAL can reduce adhesive and/or motility properties of an endothelial cell, thus interfering with vascular network formation. In some embodiments, DAL can reduce adhesive and/or motility properties of a cancer cell, thus interfering with migration and/or invasion of the cancer cell. Accordingly, methods or compositions for decreasing adhesion or motility of at least one cell (e.g., endothelial cell or cancer cell) are also provided herein. For example, such method can comprise contacting a cell with a composition comprising an anti-vascular compound described herein. As used herein, the term “contacting” generally refers to any means to deliver the composition described herein to a cell to be treated. By way of example only, in some embodiments where the cell is cultured in vitro or ex vivo, the cell can be contacted with the composition described herein by directly adding the composition into the culture medium in which the cell is cultured. In some embodiments where the cell is present in vivo, e.g., in a subject, the cell can be contacted with the composition described herein by administering a subject in need thereof the pharmaceutical composition described herein. The administration can be systemic or local.

In some embodiments, the inventors have also demonstrated that DAL can inhibit or reduce activity of an immunomodulatory enzyme (e.g., indoleamine 2,3-dioxygenase (IDO)). Accordingly, methods for increasing activity of an immune system in a subject comprising administering to a subject in need thereof the compound or immunomodulatory composition described herein are also provided herein. In some embodiments where the subject is diagnosed with or at risk of having cancer or metastatic tumor, increasing the activity of the immune system in the subject can produce an anti-tumor effect, e.g., reducing or inhibiting tumor growth by at least about 10% or more, as compared to a treatment without the composition described herein, and/or reducing or inhibiting metastatic spread by at least about 10%, as compared to a treatment without the composition described herein.

In some embodiments of any aspects described herein, the methods can further comprise selecting a subject in need thereof prior to administration of the compound and/or composition described herein. For example, the subject in need thereof can be diagnosed with or at risk of having a condition involving abnormal vasculatures described herein. In some embodiments of any aspects described herein, the subject amenable to the methods and/or compositions can have undergone treatment for the condition involving abnormal vasculatures described herein or is now receiving treatment involving abnormal vasculatures described herein. In some embodiments of any aspects described herein, the subject amenable to the methods and/or compositions can have the disease or condition described herein in remission. In some embodiments of any aspects described herein, the subject amenable to the methods and/or compositions can be previously treated for the condition described herein but did not respond to the previous treatment.

In some embodiments of any aspects described herein, the composition administered to a subject in need thereof (e.g., a subject diagnosed with or at risk of having a condition involving abnormal vasculatures) comprises at least one compound of formula (I) as described herein, including 1, 2, 3, 4, 5, or more compounds of formula (I) as described herein. In some embodiments, the compound of formula (I) can include dehydro-alpha-lapachone, or an analog, derivative, isomer, prodrug, or pharmaceutically acceptable salt thereof. In one embodiment of any aspects described herein, the composition administered to subject in need thereof (e.g., a subject diagnosed with or at risk of having a condition involving abnormal vasculatures) comprises dehydro-alpha-lapachone. Accordingly, in one embodiment, the method provided herein, for treating or preventing a condition involving abnormal vasculatures, comprises administering to a subject an anti-vascular composition comprising dehydro-alpha-lapachone or an analog, derivative, isomer, prodrug, or pharmaceutically acceptable salt thereof.

By “treating” or “treatment” of a disease or disorder is meant preventing the progression of the disorder or the condition described herein, altering the course of the disorder (for example, but are not limited to, slowing the progression of the disorder), reversing one or more symptoms of the disorder, or reducing one more symptoms, and/or one or more biochemical markers in a subject, preventing one or more symptoms from worsening or progressing, promoting recovery or improving prognosis. In some embodiments, the term “treating” as used herein can refer to prolonging survival as compared to expected survival if not receiving treatment. Thus, one of skill in the art realizes that a treatment can improve the disorder, but may not be a complete cure for the disorder. In some embodiments, the therapeutic treatment can refer to improved at least one function of an organ affected by the condition after administration of the composition described herein. In another embodiment, the therapeutic treatment can refer to alleviation or reduction of at least one symptom associated with the condition, e.g., angiogenic- or vascular-associated diseases. Measuring lessening includes any statistically significant decline in a measurable marker or symptom associated with the condition. In some embodiments, at least one symptom associated with a disorder or condition described herein can be alleviated or reduced by at least about 5%, at least about 10%, at least about 20%, at least about 30%, at least about 40%, at least about 50%, at least about 60%, at least about 70% or higher, relative to a subject without the administration of a composition described herein. In some embodiments, at least one symptom of associated with a disorder or condition described herein can be alleviated by at least about 80%, at least about 90%, at least about 95%, at least about 98% or higher, relative to a subject without the administration of a composition described herein. In one embodiment, at least one symptom associated with a disorder or condition described herein is alleviated by 100%, i.e. symptom-free. By way of example only, if the condition involving abnormal vasculatures is cancer, a decrease in tumor growth or size, or a decrease in the level of at least one tumor biomarker corresponding to the cancer can be measured, e.g., using any art-recognized methods known in the art, after administration of the composition described herein. If the condition involving abnormal vasculatures relates to a metastatic tumor, a reduction or inhibition of metastatic spread (e.g., presence of at least one metastasis and/or the number of metastases in an organ distant from the site of the primary tumor) can be detected and/or monitored, e.g., by any art-recognized imaging methods such as X-ray, CT scan, and/or MRIs, after administration of the composition described herein. In such embodiments, a decrease in the primary tumor growth or size, or a decrease in the level of at least one tumor biomarker corresponding to the metastatic cancer can also be measured, e.g., using any art-recognized methods known in the art, after administration of the composition described herein. If the condition involving abnormal vasculatures is neovascular disease of the eye, e.g., diabetic retinopathies, a decrease in the retinal swelling can be monitored by a skilled practitioner, e.g., with optical coherence tomography, after administration of the composition described herein. If the condition involving abnormal vasculatures is inflammatory arthritis, a decrease in inflammatory responses, e.g., a decrease in the number of white blood cells in the blood, after administration of the composition described herein can be determined by one of skill in the art. Methods to determine the treatment efficiency for any of the angiogenic- or vascular-associated diseases are well established in the art.

As used herein, the term “preventing” with respect to a condition or disorder refers to delaying or preventing the onset of such disorder or condition described herein, e.g., in a subject at risk of having the condition. In some embodiments, “preventing” a condition can also encompass inhibiting, decreasing, or slowing the progression or severity of the condition, e.g., in a subject being diagnosed with the condition. The onset, the progression or severity of such disorder or condition can be determined by detecting an increase in at least one symptom associated with the condition, or a decrease in the function of the organ affected by the condition. Such detection methods for any angiogenic- or vascular-associated disorder have been well-established in the art, e.g., by imaging (e.g., X-ray, MRI, CT scan, or angiography), and blood test for measuring expression levels of disorder-specific biomarkers.

In some embodiments, the method described herein can comprise administering to a subject in need thereof an effective amount of a composition or the compound of formula (I) described herein. The compositions or the compounds as disclosed herein can be administered in prophylatically or therapeutically effective amounts. A “prophylatically effective amount” means an amount necessary, at least partly, to attain the desired effect, or to delay the onset of, inhibit the progression of, or halt altogether, the onset or progression of the particular disease or disorder being treated. For example, a prophylatically effective amount of a compound or a composition comprising the compound can be an amount sufficient to inhibit or delay the onset of an angiogenic- or vascular-associated condition described herein. In some embodiments, a prophylatically effective amount of a compound or a composition comprising the compound can be an amount sufficient to inhibit or delay the progression or severity of an angiogenic- or vascular-associated condition described herein.

The phrase “therapeutically effective amount” as used herein refers to an amount of a compound described herein, or a composition comprising the compound, which is effective for producing some desired therapeutic effect in at least a sub-population of cells in a subject at a reasonable benefit/risk ratio applicable to any medical treatment. For example, a therapeutically effective amount of a compound or a composition comprising the compound can be an amount sufficient to produce a statistically significant, measurable change in at least one symptom of an angiogenic- or vascular-associated condition described herein.

Determination of a prophylatically or therapeutically effective amount is well within the capability of those skilled in the art. Generally, a prophylatically or therapeutically effective amount can vary with the subject's history, age, condition, sex and risk factors, severity and type of the medical condition in the subject, and administration of other pharmaceutically active agents. Such amounts will depend on the particular condition being treated, the status of the condition and individual patient parameters including age, physical condition, size, weight, risk factors, and concurrent treatment. These factors are well known to those of ordinary skill in the art and can be addressed with no more than routine experimentation. In some embodiments, a maximum dose, that is, the highest safe dose according to sound medical judgment, can be used. In some embodiments, a lower dose or tolerable dose can be administered for medical reasons, psychological reasons or for virtually any other reasons. It is also well within the skill of the art to either start doses of the compound at levels lower than required to achieve the desired therapeutic effect and to gradually increase the dosage until the desired effect is achieved, or start doses of the compound at high levels and to gradually decrease the dosage until the desired effect is achieved, as appropriate for the care of the individual patient.

In some embodiments, the effective amount can be sufficient to decrease or inhibit angiogenesis by at least about 10%, at least about 20%, at least about 30%, at least about 40%, at least about 50%, at least about 60%, at least about 70%, at least about 80%, at least about 90%, at least about 95%, at least about 98%, at least about 99% or higher, as compared to a reference. In other embodiments, the effective amount can be sufficient to decrease the density of existing blood vessels (e.g., in a subject with an abnormal vascular density relative to that of a normal healthy person) by at least about 10%, at least about 20%, at least about 30%, at least about 40%, at least about 50%, at least about 60%, at least about 70%, at least about 80%, at least about 90%, at least about 95%, at least about 98%, at least about 99% or higher, as compared to a reference. As used herein, the term “reference,” in some embodiments, can refer to extent of angiogenesis or vascular density measured from one or a group of normal healthy subjects. In some embodiments, the reference can be measured from one or a group of subjects having a comparable condition but without administration of the composition or the compound described herein. In some embodiments, the reference can be a previous measurement obtained from the same subject being treated. A skilled artisan can readily employ different art-recognized medical imaging methods to detect angiogenesis or vascular density associated with various conditions described herein. By way of example only, optical coherence tomography can be used to detect angiogenesis or vascular density of the retina; other medical imaging methods such as X-ray or angiography can be used to visualize blood vessels in any tissues.

In some embodiments, the effective amount can be sufficient to reduce or inhibit adhesive property of a cell to be treated by at least about 10%, at least about 20%, at least about 30%, at least about 40%, at least about 50%, at least about 60%, at least about 70%, at least about 80%, at least about 90%, at least about 95%, at least about 98%, at least about 99% or higher, as compared to a cell treated without the compound and/or composition described herein. In such embodiments, the adhesive property of a cell can be evaluated in vitro based on the morphology of a cell (e.g., round vs. extended) and/or its resistance to a shear flow. Methods for evaluating adhesive property of a cell in vitro are known to a skilled artisan, e.g., using microscopic methods and/or shear assays.

In other embodiments, the effective amount can be sufficient to reduce or inhibit motility of a cell to be treated by at least about 10%, at least about 20%, at least about 30%, at least about 40%, at least about 50%, at least about 60%, at least about 70%, at least about 80%, at least about 90%, at least about 95%, at least about 98%, at least about 99% or higher, as compared to a cell treated without the compound and/or composition described herein. In such embodiments, the motility of a cell can be evaluated in vitro using any art-recognized methods such as wound healing assays, transwell migration assays and/or real-time imaging methods.

In some embodiments, the effective amount can be sufficient to increase activity of an immune system in a subject to be treated by at least about 10%, at least about 20%, at least about 30%, at least about 40%, at least about 50%, at least about 60%, at least about 70%, at least about 80%, at least about 90%, at least about 95%, at least about 98%, at least about 99% or higher, as compared to a cell treated without the compound and/or composition described herein. In one embodiment, the effective amount can be sufficient to decrease the activity and/or expression level of an immunomodulatory enzyme (e.g., indoleamine-pyrrole 2,3-dioxygenase (IDO)) by at least about 10%, at least about 20%, at least about 30%, at least about 40%, at least about 50%, at least about 60%, at least about 70%, at least about 80%, at least about 90%, at least about 95%, at least about 98%, at least about 99% or higher, as compared to a treatment without the compound and/or composition described herein. Methods for measuring the activity of an immunomodulatory enzyme (e.g., indoleamine-pyrrole 2,3-dioxygenase (IDO)) in vitro is known in the art, e.g., using the method described in the Examples. Methods for measuring expression level (e.g., protein or mRNA/gene level) of an immunomodulatory enzyme (e.g., indoleamine-pyrrole 2,3-dioxygenase (IDO) are readily appreciated by a person having ordinary skill in the art, e.g., by immunoassay such as western blot or ELISA, and/or PCR methods including quantitative PCR.

In some embodiments, the effective amount of the composition or the compound described herein can be sufficient to decrease or reduce at least one symptom associated with an angiogenic- or vascular-associated condition by at least about 10%, at least about 20%, at least about 30%, at least about 40%, at least about 50%, at least about 60%, at least about 70% or higher, relative to that of a subject without the administration of the composition or the compound described herein, or a previous measurement obtained from the same subject being treated. In some embodiments, at least one symptom of associated with a disorder or condition described herein can be decreased by at least about 80%, at least about 90%, at least about 95%, at least about 98% or higher, relative to that of a subject without the administration of the composition or the compound described herein, or a previous measurement obtained from the same subject being treated. One of skill in the art is able to measure or diagnose the symptoms in accordance with each condition or disorder described herein.

In various embodiments, the effective amount of the composition or the compound described herein can vary from micrograms per kg of body weight to milligrams per kg of body weight. In some embodiments, the effective amount of the composition or the compound described herein can vary from about 0.001 μg/kg to 1000 μg/kg, from about 0.01 μg/kg to about 500 μg/kg, from about 0.1 μg/kg to about 100 μg/kg or from about 1 μg/kg to about 50 μg/kg. In some embodiments, the effective amount of the composition or the compound described herein can vary from about 0.001 mg/kg to about 500 mg/kg, from about 0.01 mg/kg to about 250 mg/kg, from about 0.1 mg/kg to about 150 mg/kg, from about 1 mg/kg to about 100 mg/kg, from about 5 mg/kg to about 75 mg/kg, or from about 10 mg/kg to about 50 mg/kg. In one embodiment, the effective amount of the composition or the compound described herein is between about 10 mg/kg and about 50 mg/kg. While the effective amount of the composition or the compound described herein is expressed in weight per kg of body weight, it should be understood that the effective amount can also be readily expressed in moles or molar concentrations accordingly with a known molecular weight of the compound and a pre-determined solution volume. For example, in some embodiments, the effective amount of the composition or the compound described herein can vary from 0.001 μM to about 50 μM, from about 0.01 μM to about 40 μM, from about 0.1 μM to about 20 μM, or from about 1 μM to about 10 μM.

In accordance with various embodiments provided herein, a subject in need thereof can be subjected to the treatment method described herein for any period of time. In some embodiments, the composition described herein can be administered to a subject for at least about 1 day, at least about 2 days, at least about 3 days, at least about 4 days, at least about 5 days, at least about 6 days, at least about 1 week, at least about 2 weeks, at least about 3 weeks, at least about 1 month or longer. In some embodiments, the composition described herein can be administered to a subject for at least about at least 2 months, at least about 3 months, at least about 4 months, at least about 5 months, at least about 6 months, at least about 7 months, at least about 8 months, at least about 9 months, at least about 10 months, at least about 11 months, at least about 12 months or longer. In other embodiments, the composition described herein can be administered to a subject in need thereof for at least about 1 year, at least about 2 years, at least about 3 years, at least about 4 years, at least about 5 years, at least about 6 years, or longer. In some embodiments, the composition described herein can be administered to a subject in need thereof until the condition is treated. In some embodiments, a subject in need thereof can continue to receive treatment for any period of time, e.g., days, weeks, months or years, after the condition or disorder is in remission. In such embodiment, the subject in need thereof can be administered with a lower dose of the composition after the condition is in remission. In some embodiments, a subject in need thereof can receive life-time administration of the composition described herein.

The frequency or the schedule of administering to a subject in need thereof a composition described herein can vary with various factors, including, but not limited to, modes of administration (e.g., oral or intravenous administration), dosage, severity of the condition to be treated, types of the condition to be treated, treatment type (e.g., preventive treatment or therapeutic treatment), and/or treatment regimen (e.g., administration of the composition alone or in combination with other clinical treatments). Accordingly, the time interval between any two consecutive administrations can vary, e.g., between hours, days, weeks, or months. In some embodiments, the time interval between any two consecutive administrations can be at least about 4 hours, at least about 6 hours, at least about 12 hours, at least about 24 hours, at least about 2 days, at least about 3 days, at least about 4 days, at least about 5 days, at least about 6 days, at least about 7 days or longer. In some embodiments, the time interval between any two consecutive administrations can be at least about 1 week, at least about 2 weeks, at least about 3 weeks, at least about 4 weeks or longer. In some embodiments, the time interval between any two consecutive administrations can be at least about 1 month, at least about 2 months, at least about 3 months, at least about 4 months, at least about 5 months, at least about 6 months or longer.

By way of example only, in some embodiments, the composition can be administered in pulses. Pulse therapy generally refers to a short, intensive administration of chemotherapy, usually given at intervals such as weekly or monthly. Individual pulses can be delivered to a patient continuously over a period of several hours, such as about 2 hours, 4 hours, 6 hours, 8 hours, 10 hours, 12 hours, 14 hours or 16 hours, or several days, such as 2 days, 3 days, 4 days, 5 days, 6 days, or 7 days. In some embodiments, individual pulses can be delivered to a patient continuously from about 1 hour to about 24 hours. In some embodiments, individual pulses can be delivered to a patient continuously from about 3 hours to about 9 hours.

In such embodiments, the time interval between pulses or the interval of no delivery can be greater than 24 hours or greater than 48 hours, and can be for even longer such as for 3 days, 4 days, 5 days, 6 days, 7 days, 8 days, 9 days or 10 days, two, three or four weeks or even longer. As the results achieved can vary, the time interval between pulses, when necessary, can be determined by one of ordinary skill in the art. Generally, the time interval between pulses can be determined based on the pharmacokinetics of the compound (e.g., half-life of the compound or the composition described herein). In some embodiments, another dose (pulse) of the composition can be administered when the composition or the active component of the composition from the previous dose falls below an effective range, e.g., the concentration of the compound in the blood or plasma is decreased by at least about 40%, at least about 50%, at least about 60%, at least about 70%, at least about 80%, at least about 90%, or higher, as compared to the initial concentration of the compound in the blood or plasma after administration. In some embodiments, another dose (pulse) of the composition can be administered when the composition or the compound from the previous dose is no longer detectable in the patient (e.g., the patient's blood sample). For example, the time intervals between pulses can be at least about the half-life of the composition or the compound disclosed herein, including at least about 2 times the half-life, at least about 3 times the half-life, at least about 4 times the half-life, at least about 5 times the half-life, at least about 10 times the half-life or longer, of the composition or the compound described herein.

The number of pulses in a single therapeutic regimen can be as little as two, but is typically from about 5 to 10, 10 to 20, 15 to 30 or more. In some embodiments, patients can receive the compositions or the compound described herein for life according to the methods provided herein. Compositions can be administered by any means, e.g., delivered to the patient as an injection (e.g. intravenous, subcutaneous, intra-arterial), infusion or instillation. Various methods and apparatus for pulsing compositions by infusion or other forms of delivery to the patient are disclosed in, but are not limited to, U.S. Pat. Nos. 4,747,825; 4,723,958; 4,948,592; 4,965,251 and 5,403,590.

In alternative embodiments, compositions comprising the compound described herein can be administered by sustained release, i.e., the composition or the compound described herein is released into the body slowly over an extended period of time. Sustained release can be accomplished by means of an osmotic pump or other delivery systems as described later. In such embodiments, the composition or the compound can be administered over any period of time, such as at least about 2 days, at least about 3 days, at least about 4 days, at least about 5 days, at least about 6 days, at least about 7 days, at least about 2 weeks, at least about 3 weeks, at least about 4 weeks, at least about 1 month, at least about 2 months, at least about 3 months or longer.

In some embodiments, a patient can be administered with a bolus of the composition described herein every 2 hours, every 4 hours, every 6 hours, every 12 hours, every 24 hours, every 2 days, every 3 days, every 4 days, every 5 days, every 6 days, every week or longer. Such administration can be performed by injection or orally. The term “bolus” as used herein refers to a single dose that is administered to a subject in less than 10 minutes, less than 5 minutes, less than 3 minutes, or shorter. The term “bolus” is generally intended to exclude dosage forms such as sustained release, pulsed release, and time release.

Some embodiments of any methods described herein can also encompass combination therapy, in which the compound is administered in conjunction with at least one active agent as described later.

The compound described herein and at least one active agent can be administrated to a subject in the same pharmaceutical composition or in different pharmaceutical compositions (at the same time or at different times). When administrated at different times, the compound and the active agent can be administered within 5 minutes, 10 minutes, 20 minutes, 60 minutes, 2 hours, 3 hours, 4, hours, 8 hours, 12 hours, 24 hours, 2 days, 3 days, 4 days, 5 days, 6 days, 1 week, or longer of administration of the other. When the compound and the active agent are administered in different pharmaceutical compositions, routes of administration can be different.

Some embodiments described herein can encompass combination therapy in which the compositions described herein can be used in combination or conjunction with therapeutics, physiotherapy and/or behavioral psychotherapy used in the treatment of various angiogenic- or vascular-associated conditions described herein, e.g., but not limited to, rheumatoid arthritis, obesity, endometriosis, and Alzheimer's disease.

By way of example only, for treatments of rheumatoid arthritis, there are therapeutic drugs that decrease pain and local inflammation including aspirin and non-steroidal anti-inflammatory drugs or NSAIDS (such as ibuprofen or naproxen) and other immunosuppressive drugs that decrease pain and inflammation while decreasing the growth of abnormal synovial tissue (the tissue that lines the inside of the joint). These drugs include methotrexate and low doses of corticosteroids (such as prednisone or cortisone). Other medications used to treat rheumatoid arthritis include: anti-malarial medications (such as hydroxychloroquine), gold, sulfasalazine, penicillamine, cyclophosphamide, cyclosporine, minocycline, interleukin receptor antagonist and anti-IL2 antibodies.

Treatment for Alzheimer's disease include, but are not be limited to, nonsteroidal anti-inflammatory drugs (NSAIDs), estrogen, steroids such as prednisone, vitamin E, menantine, donepezil, rivastigmine, tacrine, and galantamine. Holistic medicine include example such as gingko nuts extracts.

Treatment of endometriosis include, but should not be construed as limited to, a combination oral contraceptives (estrogen plus a progestin), progestins (such as medroxyprogesterone, danazol (a synthetic hormone related to testosterone, gonadotropin-releasing hormone agonists (GnRH agonists such as buserelin, goserelin, leuprolide and nafarelin), and nonsteroidal anti-inflammatory drugs (NSAIDs) for pain control.

Examples of treatment options for obesity include dieting and nutritional counseling, exercise regime, gastric-bypass surgery, and drugs such as a combination of fenfluramine and phentermine (often called fen-phen), orlistat, sibutramine, phentermine, benzphetamine, diethylpropion, mazindol, and phendimetrazine.

In particular embodiments, the compositions as disclosed herein can be used for administration in conjunction therapies used for treatment of diseases associated with vascular permeability, such as vascular complications of diabetes such as non-proliferative diabetic retinopathy and nephropathy, nephrotic syndrome, pulmonary hypertension, burn edema, tumor edema, brain tumor edema, IL-2 therapy-associated edema, and other edema-associated diseases, as disclosed in International Application No: WO2003/086178 and U.S. Patent Applications US2005/0203013 and US2005/0112063 which are incorporated herein in their entirety by reference. In a particular embodiment, the formulations and compositions as disclosed herein are particularly useful for administration in conjunction with IL-2 therapy, where the limiting factor of IL-2 therapy is IL-2 therapy-associated edema as disclosed in International Application No: WO2003/086178 and U.S. Patent Applications US2005/0203013 and US2005/0112063.

In some embodiments, the methods can be used in conjunction with any treatment for cancer, including, but not limited to, surgery (e.g., to remove a tumor), radiation therapy, photodynamic therapy, chemotherapy, immunotherapy, and any combinations thereof.

### Compounds of Formula (I)

Regulation of blood vessel growth has proven to be an important strategy in the treatment of cancer, and may provide new targets for the treatment of inflammatory disorders, asthma, obesity, diabetes, multiple sclerosis, endometriosis, and bacterial infections (1-2).

Cell adhesion pathways present attractive targets for antivascular agents in cancer therapy. Endothelial cell (EC) adhesion is crucial for blood vessel function (8-9). Blood vessels in tumors are abnormal, featuring unusual leakiness, high tortuosity, and inefficient network structure (10). The immature ECs of this abnormal neovasculature in tumors have weak cell-cell junctions (10-13)—likely making them particularly sensitive to anti-adhesion therapies. Candidate anti-adhesion agents for antivascular therapy have been identified, and have shown promising results in preclinical studies (14). Unfortunately, these early attempts at development of anti-adhesion agents for antivascular therapy were met with failure stemming from issues with toxicity. In accordance with embodiments described herein, a non-toxic compound targeting cell adhesion is provided herein, which can produce a safer antivascular effect specific to neovasculature.

In various embodiments described herein, the compounds of formula (I), and analogs, derivatives, isomers, prodrugs, and pharmaceutically salt thereof, have anti-vascular activity, wherein the formula (I) has a structure:

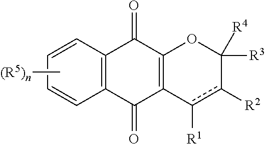

In one embodiment, the compound is of formula (I). In another embodiment, the compound is an isomer of formula (I). In still another embodiment, the compound is an analog of formula (I). In yet another embodiment, the compound is a prodrug of a compound of formula (I). In another embodiment, the compound is a pharmaceutically acceptable salt of a compound of formula (I).

In compounds of formula (I), R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted. In some embodiments, R¹ and R² can be independently hydrogen, halogen or alkyl. In one embodiment, R¹ and R² are both hydrogen.

In embodiments of formula (I), the dotted line between the carbons to which R¹ and R² are bonded represents an optional double bond.

In compounds of formula (I), R³ and R⁴ are each independently selected from hydrogen, alkyl, alkenyl, cyclyl, heterocyclyl, aryl, heteroaryl, each of which can be optionally substituted. In some embodiments, R³ and R⁴ are independently hydrogen, hydrogen or alkyl. In some embodiments, R³ and R⁴ are both alkyl. In one embodiment, R³ and R⁴ are both methyl.

In compounds of formula (I), n can be 0, 1, 2, 3, or 4.

In some embodiments, n is 0.

In some compounds of formula (I), when n is greater than 0 (i.e., n=1, 2, 3 or 4), R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted. In some embodiments, R⁵ can be independently alkyl, e.g., methyl, or ethyl. In some embodiments, R⁵ can be halogen.

In some compounds of formula (I), R⁶ is independently selected for each occurrence, hydrogen, alkyl, alkenyl, alkynyl, cyclyl, heterocyclyl, aryl, or heteroaryl, each of which can be optionally substituted. In some embodiments, R⁶ is hydrogen. In some embodiments, R⁶ is alkyl.

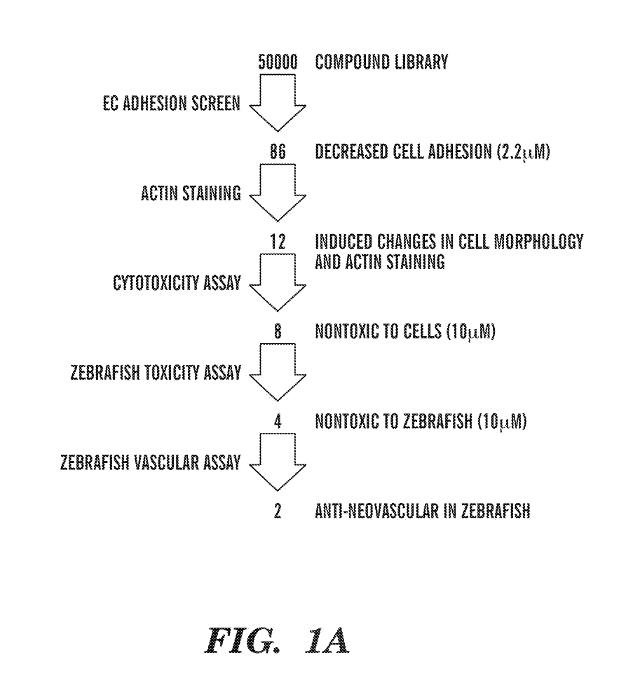

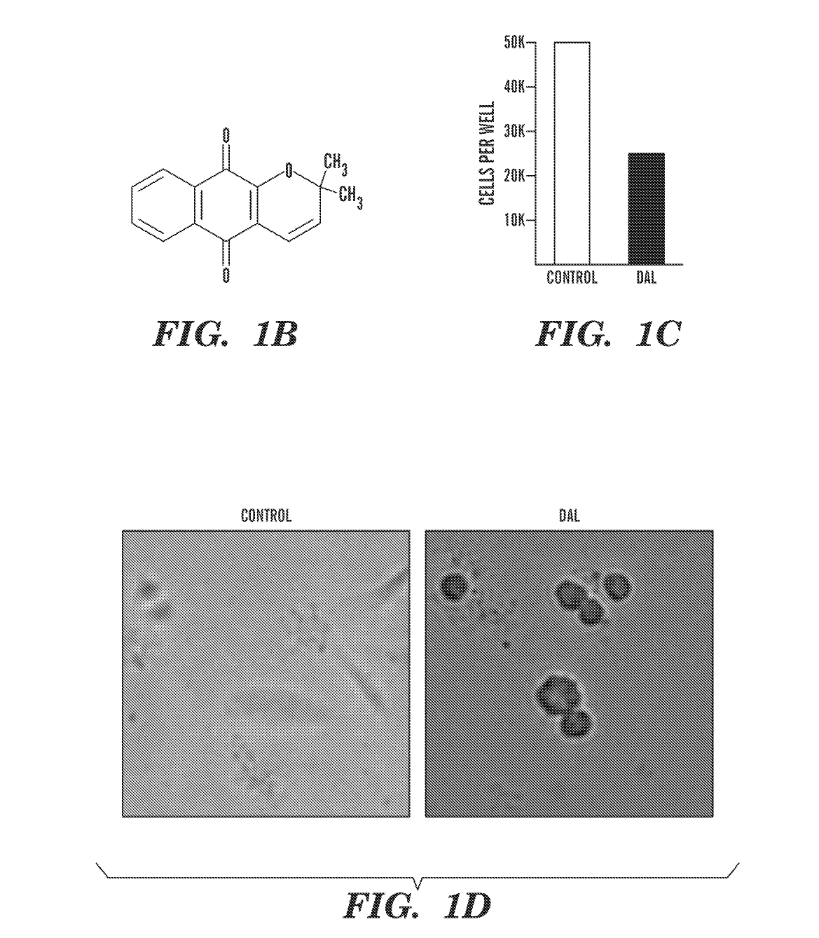

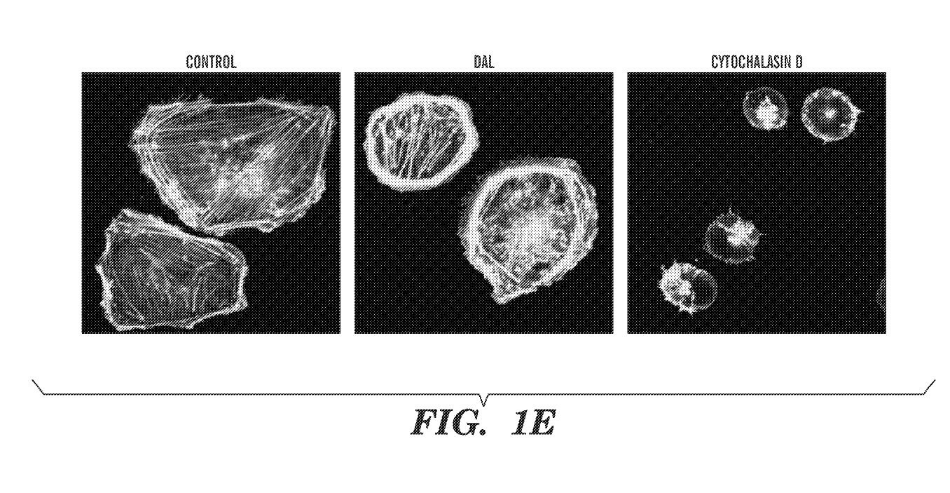

In one embodiment of compounds of formula (I), the dotted line between the carbons to which R¹ and R² are bonded forms a double bond; R¹ and R² are both hydrogen; R³ and R⁴ are both alkyl, e.g., methyl; and n is 0. By way of example only, an exemplary embodiment of such compound is shown in FIG. 1B.

In some embodiments, the compound of formula (I) is a product derived from Tabebuia Avellaneda tree. In such embodiments, the compound of formula (I) is dehydro-alpha-lapachone. In one embodiment, the compound of formula (I) is an analog of dehydro-alpha-lapachone. In one embodiment, the compound of formula (I) is a derivative of dehydro-alpha-lapachone. In one embodiment, the compound of formula (I) is an isomer of dehydro-alpha-lapachone. In one embodiment, the compound of formula (I) is a prodrug of dehydro-alpha-lapachone. In one embodiment, the compound of formula (I) is a pharmaceutically salt of dehydro-alpha-lapachone.

In accordance with embodiments described herein, compounds of formula (I) have anti-vascular and/or anti-angiogenic activity. The term “anti-vascular activity” is used herein in reference to compounds capable of inhibiting or delaying formation or growth of blood vessels, including newly-formed blood vessels, existing blood vessels, mature blood vessels, capillaries and any vessel branching, and/or decreasing the vascular density, e.g., the density of existing blood vessels. In some embodiments, the term “anti-vascular activity” can encompass an ability to inhibit or decrease anastomosis, which is connection of at least two blood vessels. In some embodiments, the term “anti-vascular activity” can include an ability to inhibit or decrease maturation of a newly-formed blood vessel.

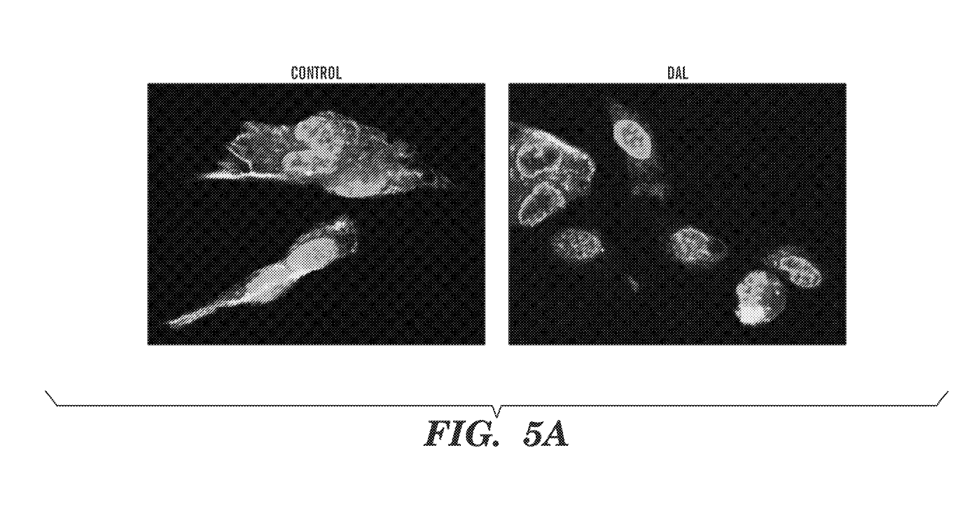

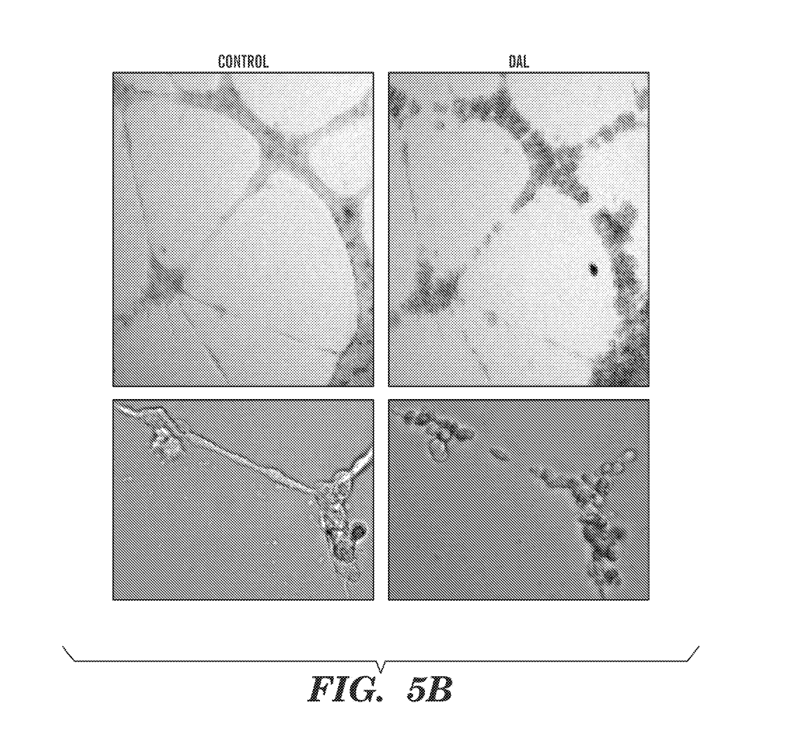

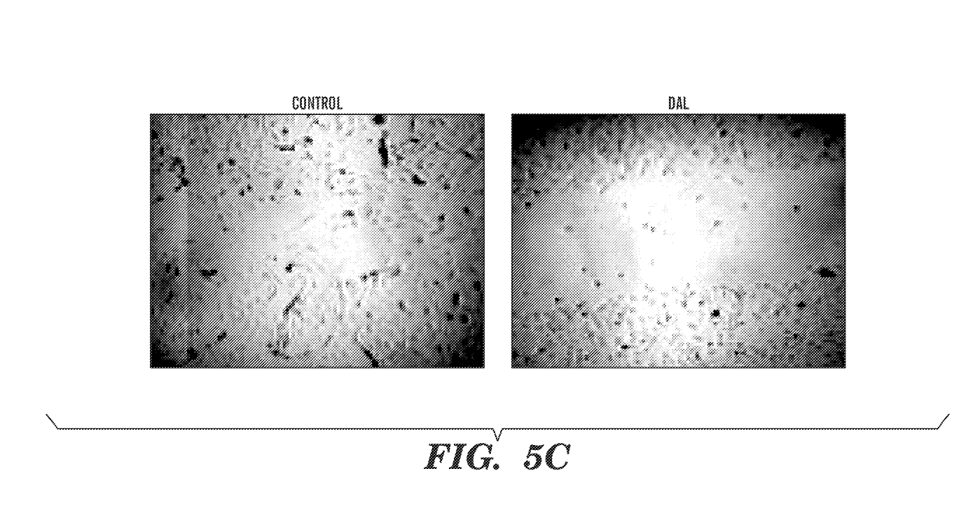

Methods for measuring anti-vascular and/or anti-angiogenic activity can be performed in vitro and/or in vivo, and are well known in the art. For example, in vitro methods such as monitoring the formation or disruption of cordlike network by endothelial cells (e.g., HUVECs) seeded in Matrigel in the presence of a candidate compound can be employed. An anti-vascular and/or anti-angiogenic compound will disrupt the existing cordlike structures and/or inhibit formation of such network of cordlike structures as shown in FIG. 5B. For in vivo methods, different animal models such as mammals (e.g., mice, rats, and porcines), C. Elegans, or zebrafish can be used to study the effects of a compound on vasculatures or angiogenesis. Exemplary in vivo vascular assays using mice and zebrafish, in combination with imaging methods, to determine the effects of a compound on vessel development or regeneration as well as to measure various vascular parameters such as vascular volume fraction, vascular length, vascular diameter are described in the Examples.

Any art-recognized methods for determining anti-vascular activity of a compound can be used. In some embodiments, the chick chorioallantoic membrane (CAM) assay can also be used to determine if the compositions and compounds disclosed herein are anti-vascular. An exemplary CAM assay is performed by implanting a membrane, a coverslip or a gel containing the compound or the composition disclosed herein on the chick embryo chorioallantoic membrane through a hole cut in the egg shell. The subsequent incubation period can range from 1-3 days or longer, depending on the compound or the composition, after which time angiogenesis can be quantified via image analysis or colorimetric detection methods. However, this is a non-mammalian system which should be taken into consideration when interpreting results.

Other methods for identifying an anti-vascular compound or composition include, but are not limited to, the corneal micropocket angiogenesis assay (Rogers M. 2 Nature Protocols 2545 (2007)), hamster cheek pouch assay, the Matrigel assay seeded with endothelial cells (e.g., HUVEC) and modifications thereof, and a tubule formation assay in which endothelial cells are co-cultured with fibroblasts. Methods for performing the Matrigel assay and a tubule formation assay are well established and a comparison of these assays are described in Donovan et al. (4 Angiogenesis 113 (2001), incorporated herein by reference). Commercially-available in vitro angiogenesis assay kits (e.g., from Chemicon/Millipore) and the fibrin gel in vitro angiogenesis assay kits (e.g., Chemicon Catalog No. ECM630) can be used to determine anti-vascular activity of a compound or a composition described herein.

In some embodiments, the compound described herein can inhibit or delay formation or growth of blood vessels, including new or existing blood vessels or capillaries, by at least about 10%, at least about 20%, at least about 30%, at least about 40%, at least about 50%, at least about 60%, at least about 70%, at least about 80%, at least about 90%, at least about 95%, about 98%, 99% or 100% (complete inhibition), as compared to the formation of blood vessels in the absence of the compound. In some embodiments, the compound described herein can decrease vascular density, which can be determined by measuring vascular volume fraction, and vascular length and diameter, by at least about 10%, at least about 20%, at least about 30%, at least about 40%, at least about 50%, at least about 60%, at least about 70%, at least about 80%, at least about 90%, at least about 95%, about 98%, 99% or 100% (complete disruption or pruning), as compared to the vascular density in the absence of the compound.

In some embodiments, a compound described herein can further comprise anti-tumor activity. The term “anti-tumor activity” is used herein to refer to inhibiting or delaying the formation or growth of a tumor. In some embodiments, the term “anti-tumor activity” also refers to inhibit or delay the progression or malignancy of a tumor, e.g., metastasis. Methods for determining anti-tumor activity of a compound are well known in the art, e.g., the ones described in the Examples to monitor in vivo the tumor size over a period of time or the doubling time of a tumor after administration of a compound. Any art-recognized methods for measuring the anti-tumor activity of a compound can be used.

Accordingly, in some embodiments, compounds described herein can inhibit or decrease growth of a tumor (e.g., determined by tumor volume or doubling time), by at least about 10%, at least about 20%, at least about 30%, at least about 40%, at least about 50%, at least about 60%, at least about 70%, at least about 80%, at least about 90%, at least about 95%, about 98%, 99% or 100% (complete inhibition), as compared to the growth of a tumor in the absence of the compounds. In other embodiments, compounds of formula (I) can inhibit or decrease malignancy of a tumor (e.g., determined by number of metastases observed in a secondary site), by at least about 10%, at least about 20%, at least about 30%, at least about 40%, at least about 50%, at least about 60%, at least about 70%, at least about 80%, at least about 90%, at least about 95%, about 98%, 99% or 100%, as compared to malignancy of a tumor in the absence of the compounds.

In some embodiments, compounds described herein can inhibit or decrease immune tolerance to diseased cells or proteins or antigens thereof. The term “immune tolerance” as used herein refers to a lack of an immune response after exposure to a diseased cell, protein and/or an antigen thereof. In accordance with embodiments described herein, the compound (e.g., DAL) can regulate the activity of an immunomodulatory enzyme, and thus modulate the degree of immune tolerance to the diseased cells or proteins or antigens thereof. To improve efficacy of a therapeutic treatment, a decrease in the degree of immune tolerance to the diseased cells or proteins or antigens thereof is desirable. In some embodiments, a compound or a composition described herein can decrease or inhibit activity of an immunosuppressive enzyme (e.g., indoleamine 2,3-dioxygenase (IDO)), for example, by at least about 10%, including at least about 20%, at least about 30%, at least about 40%, at least about 50%, at least about 60%, at least about 70%, at least about 80%, at least about 90%, at least about 95%, about 98%, about 99% or 100%, relative to the measured activity of an immunosuppressive enzyme in the absence of the compound or composition described herein. In some embodiments, a compound or a composition described herein can increase or stimulate activity of an immunostimulatory enzyme, for example, by at least about 10%, including at least about 20%, at least about 30%, at least about 40%, at least about 50%, at least about 60%, at least about 70%, at least about 80%, at least about 90%, at least about 95%, about 98%, about 99% or 100%, relative to the measured activity of an immunosuppressive enzyme in the absence of the compound or composition described herein. In vitro methods for determining enzymatic activities, e.g., activity of immunomodulatory enzymes are well known in the art, such as using spectrophotometric assays (as described in the Examples) or fluorometric assays.

Many patients treated with current VEGF inhibitors (e.g., AVASTIN®) become refractory or acquire resistance to the VEGF inhibitors. Compounds or compositions decreasing immune tolerance as described herein can be administered in conjunction to the VEGF inhibitors. Such combination therapy can decrease the probability of a patient to acquire resistance to anti-angiogenic agents that inhibit VEGF signaling pathway (e.g., VEGF inhibitors) by at least about 10%, as compared to a patient taking VEGF inhibitors alone.

In some embodiments, a compound or composition described herein can inhibit or decrease adhesion of endothelial cells by at least about 10%, at least about 20%, at least about 30%, at least about 40%, at least about 50%, at least about 60%, at least about 70%, at least about 80%, at least about 90%, at least about 95%, about 98%, about 99% or 100%, relative to the cell adhesion measured in the absence of the compound or composition described herein. Various methods are well established for determining the effect of a drug on cell adhesion, e.g., by monitoring the cell morphology by immunochemistry and microscopy (as shown in FIGS. 1D and 1E), or by determining the number of cells adhered to a substrate in the presence of the test compound (as shown in FIG. 1C).

In some embodiments, a compound or composition described herein can inhibit or decrease motility of one or more cells (e.g., endothelial cells and/or cancer cells) by at least about 10%, at least about 20%, at least about 30%, at least about 40%, at least about 50%, at least about 60%, at least about 70%, at least about 80%, at least about 90%, at least about 95%, about 98%, about 99% or 100%, relative to the cell motility measured in the absence of the compound or composition described herein. Various methods are well established for determining the effect of a drug on cell motility or migration, e.g., transwell assays or wound healing assay as shown in FIG. 5C.

In some embodiments, a compound of formula (I) described herein can have a half-life (e.g., in plasma after administration) of at least about 30 mins, at least about 1 hour, at least about 2 hours, at least about 3 hours, at least about 4 hours, at least about 5 hours, at least about 6 hours or longer. In some embodiments, the half-life of the compound can be between 30 mins and 6 hours, or between 1 hour and 3 hours.

In some embodiments, the compound as described herein can be further modified, e.g., conjugated to a macromolecule, such as a sugar molecule (e.g., cyclodextrin) or a polymer molecule. Some embodiments comprising such conjugated compound can be used to improve the solubility when the compound described herein has low solubility, e.g., in water.

### Active Agents

Some embodiments of the methods and compositions described herein can also encompass administration of at least one active agent, for example, any art-recognized therapeutic agents used in treatment of any condition or disorder described herein, anti-cancer agents, anti-angiogenic or anti-vascular agents, anti-inflammation agents, VEGF inhibitors, antibiotics, anti-viral agents, anti-fungal agents, anti-protozoal agents, hormones, radioactive-agents, toxins, anesthetics, and any combinations thereof.

The term “therapeutic agents” is art-recognized and refers to any chemical moiety that is a biologically, physiologically, or pharmacologically active substance that acts locally or systemically in a subject. Examples of therapeutic agents, also referred to as “drugs”, are described in well-known literature references such as the Merck Index; the Physicians Desk Reference; the Pharmacological Basis of Therapeutics; the United States Pharmacopeia, The National Formulary; Goodman and Oilman's The Pharmacological Basis of Therapeutics; and Harrison's Principles of Internal Medicine (the contents of which are incorporated herein by references in their entirety), and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment. Various forms of a therapeutic agent can be used, depending on administration modes and routes.

Exemplary antibiotics that can be used in conjunction with the methods or compositions described herein include, but are not limited to, aminoglycosides (e.g., neomycin), ansamycins, carbacephem, carbapenems, cephalosporins (e.g., cefazolin, cefaclor, cefditoren, cefditoren, ceftobiprole), glycopeptides (e.g., vancomycin), macrolides (e.g., erythromycin, azithromycin), monobactams, penicillins (e.g., amoxicillin, ampicillin, cloxacillin, dicloxacillin, flucloxacillin), polypeptides (e.g., bacitracin, polymyxin B), quinolones (e.g., ciprofloxacin, enoxacin, gatifloxacin, ofloxacin, etc.), sulfonamides (e.g., sulfasalazine, trimethoprim, trimethoprim-sulfamethoxazole (co-trimoxazole)), tetracyclines (e.g., doxycyline, minocycline, tetracycline, etc.), chloramphenicol, lincomycin, clindamycin, ethambutol, mupirocin, metronidazole, pyrazinamide, thiamphenicol, rifampicin, thiamphenicl, dapsone, clofazimine, quinupristin, metronidazole, linezolid, isoniazid, fosfomycin, or fusidic acid.

Exemplary antibodies include, but are not limited to, abciximab, adalimumab, alemtuzumab, basiliximab, bevacizumab, cetuximab, certolizumab pegol, daclizumab, eculizumab, efalizumab, gemtuzumab, ibritumomab tiuxetan, infliximab, muromonab-CD3, natalizumab, ofatumumab omalizumab, palivizumab, panitumumab, ranibizumab, rituximab, tositumomab, trastuzumab, altumomab pentetate, arcitumomab, atlizumab, bectumomab, belimumab, besilesomab, biciromab, canakinumab, capromab pendetide, catumaxomab, denosumab, edrecolomab, efungumab, ertumaxomab, etaracizumab, fanolesomab, fontolizumab, gemtuzumab ozogamicin, golimumab, igovomab, imciromab, labetuzumab, mepolizumab, motavizumab, nimotuzumab, nofetumomab merpentan, oregovomab, pemtumomab, pertuzumab, rovelizumab, ruplizumab, sulesomab, tacatuzumab tetraxetan, tefibazumab, tocilizumab, ustekinumab, visilizumab, votumumab, zalutumumab, and zanolimumab.

Exemplary enzymes suitable for use herein include, but are not limited to, peroxidase, lipase, amylose, organophosphate dehydrogenase, ligases, restriction endonucleases, ribonucleases, DNA polymerases, glucose oxidase, and laccase.

Anti-cancer agents or anti-neoplastic agents include, but are not limited to, mitotic inhibitors (e.g., paclitaxel), 5-fluorouracil, 5-fluorouridine, mitomycin-C, doxorubicin, vincristine, vinblastine, cisplatin, adriamycin, tamoxifen and zotarolimus, inhibitors of matrix metalloproteinases such as marimastat, growth factor antagonists, signal transduction inhibitors and protein kinase C inhibitors.

Anti-angiogenic or anti-vascular agents refer to any small molecules, polynucleotides (including, e.g., an inhibitory RNA (RNAi or siRNA)), polypeptides, isolated proteins, recombinant proteins, antibodies or fragments, conjugates or fusion proteins thereof, that can directly or indirectly inhibit or decrease formation or growth of blood vessels; reduce or alleviate vascular hyperproliferation or hyperpermeability; and/or decrease vascular density (e.g., number of blood vessels and/or blood vessel diameter). It should be understood that the anti-angiogenic agent includes those agents that bind and block the angiogenic activity of the angiogenic factor or its receptor. Nonlimiting examples of angiogenic factors or their receptors that are involved in stimulating the development of blood vessels, e.g., promote angiogenesis, endothelial cell growth, stability of blood vessels, and/or vasculogenesis include VEGF and members of the VEGF family and their receptors (VEGF-B, VEGF-C, VEGF-D, VEGFR1, VEGFR2 and VEGFR3), P1GF, PDGF family, fibroblast growth factor family (FGFs), TIE ligands (Angiopoietins, ANGPT1, ANGPT2), TIE1, TIE2, ephrins, Bv8, Delta-like ligand 4 (DLL4), EGF-like-domain, multiple 7 (EGFL7), Del-1, fibroblast growth factors: acidic (aFGF) and basic (bFGF), FGF4, FGF9, BMP9, BMP10, Follistatin, Granulocyte colony-stimulating factor (G-CSF), GM-CSF, Hepatocyte growth factor (HGF)/scatter factor (SF), Interleukin-8 (IL-8), CXCL12, Leptin, Midkine, neuropilins, NRP1, NRP2, Placental growth factor, Platelet-derived endothelial cell growth factor (PD-ECGF), Platelet-derived growth factor, especially PDGF-BB, PDGFR-alpha, or PDGFR-beta, Pleiotrophin (PTN), Progranulin, Proliferin, Transforming growth factoralpha (TGF-alpha), Transforming growth factor-beta (TGFbeta), Tumor necrosis factor-alpha (TNF-alpha), Alkl, CXCR4, Notchl, Notch4, Sema3A, Sema3C, Sema3F, Robo4, ESM1, Perlecan, and any combinations thereof. It would also include factors that accelerate wound healing, such as growth hormone, insulin-like growth factor-I (IGF-I), epidermal growth factor (EGF), CTGF and members of its family, and TGF-alpha and TGF-beta. See, e.g., Klagsbrun and D'Amore (1991) Annu. Rev. Physiol. 53:217-39; Streit and Detmar (2003) Oncogene 22:3172-3179; Ferrara & Alitalo (1999) Nature Medicine 5(12): 1359-1364; Tonini et al. (2003) Oncogene 22:6549-6556 (e.g., Table 1 listing known angiogenic factors); and, Sato (2003) Int. J. Clin. Oncol. 8:200-206.

In some embodiments, a compound of formula (I), or an analog, derivative, isomer, prodrug, or pharmaceutically acceptable salt thereof, for example, dehydro-alpha-lapachone, is an anti-angiogenic or anti-vascular agent. Exemplary anti-angiogenic or anti-vascular agents that can be administered in combination with the methods or compositions described herein include, but are not limited to, VEGF inhibitors described below, the anti-angiogenic molecules disclosed in the U.S. Patent Application No.: US2010/0204338 and US 2011/0002848; and the International Application No.: WO 2004/113304. Other types of molecules that elicit anti-angiogenic or anti-vascular properties include, without limitations, (i) anti-mitotics such as fluorouracil, mytomycin-C, taxol; (ii) estrogen metabolites such as 2-methoxyestradiol; (iii) matrix metalloproteinase (MMP) inhibitors, which inhibit zinc metalloproteinases (metalloproteases) (e.g. betimastat, BB16, TIMPs, minocycline, GM6001, or those described in “Inhibition of Matrix Metalloproteinases: Therapeutic Applications” (Golub, Annals of the New York Academy of Science, Vol. 878a; Greenwald, Zucker (Eds.), 1999); (iv) anti-angiogenic multi-functional agents and factors such as IFNα (U.S. Pat. No. 4,530,901; U.S. Pat. Nos. 4,503,035; 5,231,176); angiostatin and plasminogen fragments (e.g. kringle 1-4, kringle 5, kringle 1-3 (O'Reilly, M. S. et al., Cell (Cambridge, Mass.) 79(2): 315-328, 1994; Cao et al., J. Biol. Chem. 271 29461-29467, 1996; Cao et al., J. Biol Chem 272: 22924-22928, 1997); endostatin (O'Reilly, M. S. et al., Cell 88(2), 277, 1997 and WO 97/15666), thrombospondin (TSP-1; Frazier, 1991, Curr Opin Cell Biol 3(5):792); platelet factor 4 (PF4); (v) plasminogen activator/urokinase inhibitors; (vi) urokinase receptor antagonists; (vii) heparinases; (viii) fumagillin analogs such as TNP-470; (ix) tyrosine kinase inhibitors such as SUI 01 (including ErbB receptor antagonists (EGFR/HER2 antagonists)); (x) suramin and suramin analogs; (xi) angiostatic steroids; (xii) VEGF and bFGF antagonists; (xiii) VEGF receptor antagonists such as anti-VEGF receptor antibodies (DC-101); (xiv) flk-1 and fit-1 antagonists; (xv) cyclooxxvagenase-II inhibitors such as COX-II; and (xvi) integrin antagonists and integrin receptor antagonists such as av antagonists and αv receptor antagonists, for example, anti-αv receptor antibodies and RGD peptides.

As used herein the term “VEGF inhibitors” refers to any compound or agent that produces a direct effect on the signaling pathways that promote growth, proliferation and survival of a cell by inhibiting the function of the VEGF protein, including inhibiting the function of VEGF receptor proteins. The term “agent” or “compound” as used herein means any organic or inorganic molecule, including modified and unmodified nucleic acids such as antisense nucleic acids, RNAi agents such as siRNA or shRNA, peptides, peptidomimetics, receptors, ligands, and antibodies. Additional VEGF inhibitors, include for example, AVASTIN® (bevacizumab), an anti-VEGF monoclonal antibody of Genentech, Inc. of South San Francisco, Calif., VEGF Trap (Regeneron/Aventis). Additional VEGF inhibitors include CP-547,632 (3-(4-Bromo-2,6-difluoro-benzyloxy)-5-[3-(4-pyrrolidin 1-yl-butyl)-ureido]-isothiazole-4-carboxylic acid amide hydrochloride; Pfizer Inc., NY), AG13736, AG28262 (Pfizer Inc.), SU5416, SU11248, & SU6668 (formerly Sugen Inc., now Pfizer, New York, N.Y.), ZD-6474 (AstraZeneca), ZD4190 which inhibits VEGF-R2 and -R1 (AstraZeneca), CEP-7055 (Cephalon Inc., Frazer, Pa.), PKC 412 (Novartis), AEE788 (Novartis), AZD-2171, NEXAVAR® (BAY 43-9006, sorafenib; Bayer Pharmaceuticals and Onyx Pharmaceuticals), vatalanib (also known as PTK-787, ZK-222584: Novartis & Schering: AG), MACUGEN® (pegaptanib octasodium, NX-1838, EYE-001, Pfizer Inc./Gilead/Eyetech), 1M862 (glufanide disodium, Cytran Inc. of Kirkland, Wash., USA), VEGFR2-selective monoclonal antibody DC101 (ImClone Systems, Inc.), angiozyme, a synthetic ribozyme from Ribozyme (Boulder, Colo.) and Chiron (Emeryville, Calif.), Sirna-027 (an siRNA-based VEGFR1 inhibitor, Sirna Therapeutics, San Francisco, Calif.) Caplostatin, soluble ectodomains of the VEGF receptors, Neovastat (IEterna Zentaris Inc; Quebec City, Calif.), ranibizumab, thalidomide, and AGM-1470 (TNP-470), a synthetic analog of fumagillin (alternate names: Amebacilin, Fugillin, Fumadil B, Fumadil) (A. G. Scientific, catalog #F1028), an angio-inhibitory compound secreted by Aspergillus fumigates, and any combinations thereof.

In some embodiments, VEGF inhibitors that can be used in combination with the compound and/or composition described herein for treatment of an ocular disease or disorder involving abnormal vasculatures can include, but are not limited to, an anti-VEGF aptamer (pegaptanib, MACUGEN®); a Fab fragment of a monoclonal antibody directed against VEGF-A (ranibizumab, LUCENTIS® or bevacizumab, AVASTIN®); or a combination thereof.

Representative examples of antibiotics include, but are not limited to, penicillins; cephalosporins such as cefadroxil, cefazolin, cefaclor; aminoglycosides such as gentamycin and tobramycin; sulfonamides such as sulfamethoxazole; and metronidazole.

Representative examples of anti-inflammation agents include, but are not limited to, steroids such as prednisone, prednisolone, hydrocortisone, adrenocorticotropic hormone, and sulfasalazine; and non-steroidal anti-inflammatory drugs (“NSAIDS”) such as aspirin, ibuprofen, naproxen, fenoprofen, indomethacin, and phenylbutazone.

Representative examples of antiviral agents include, but are not limited to, acyclovir, ganciclovir, zidovudine. Representative examples of antifungal agents include, but are not limited to, nystatin, ketoconazole, griseofulvin, flucytosine, miconazole, clotrimazole. Representative examples of antiprotozoal agents include, but are not limited to, pentamidine isethionate, quinine, chloroquine, and mefloquine.

Non-limiting examples of hormones include thyroid hormone, estrogen, progesterone, cortisone and/or growth hormone, other biologically active molecules such as insulin, as well as TH1 (e.g., Interleukins-2, -12, and -15, gamma-interferon) or TH2 (e.g., Interleukins-4 and -10) cytokines.

Additional examples of active agent include, but are not limited to, a-adrenergic blocking agents, angiotensin II receptor antagonists and receptor antagonists for histamine, serotonin, endothelin; inhibitors of the sodium/hydrogen antiporter (e.g. amiloride and its derivatives); agents that modulate intracellular Ca²⁺ transport such as L-type (e.g., diltiazem, nifedipine, verapamil) or T-type Ca²⁺ channel blockers (e.g., amiloride), calmodulin antagonists (e.g., H7) and inhibitors of the sodium/calcium antiporter (e.g., amiloride); ap-1 inhibitors (for tyrosine kinases, protein kinase C, myosin light chain kinase, Ca²⁺/calmodulin kinase II, casein kinase II); anti-depressants (e.g. amytriptyline, fluoxetine, LUVOX® and PAXIL®); cytokine and/or growth factors, as well as their respective receptors, (e.g., the interleukins, α, β or γ-IFN, GM-CSF, G-CSF, epidermal growth factor, transforming growth factors alpha and beta, TNF, and antagonists of vascular epithelial growth factor, endothelial growth factor, acidic or basic fibroblast growth factors, and platelet derived growth factor); inhibitors of the IP3 receptor (e.g., heparin); protease and collagenase inhibitors (e.g., TIMPs, discussed above); nitrovasodilators (e.g., isosorbide dinitrate); anti-mitotic agents (e.g., colchicine, anthracyclines and other antibiotics, folate antagonists and other anti-metabolites, vinca alkaloids, nitrosoureas, DNA alkylating agents, topoisomerase inhibitors, purine antagonists and analogs, pyrimidine antagonists and analogs, alkyl sulfonates); immunosuppressive agents (e.g., adrenocorticosteroids, cyclosporine); sense or antisense oligonucleotides (e.g., DNA, RNA, nucleic acid analogues (e.g., peptide nucleic acids) or any combinations of these); and inhibitors of transcription factor activity (e.g., 1-(4-hexyphenyl)-2-propane-1-one, elbfluorene, pifithrin-α and derivatives thereof, STAT3 inhibitor, STATS inhibitor, Tanshinone IIA).

Exemplary radioactive agents include, but are not limited to, Cu-64, Ga-67, Ga-68, Zr-89, Ru-97, Tc-99m, Rh-105, Pd-109, In-111, I-123, I-125, I-131, Re-186, Re-188, Au-198, Au-199, Pb-203, At −211, Pb-212 and Bi-212). Exemplary toxins include, without limitations, (e.g., ricin, abrin, diphtheria toxin, cholera toxin, gelonin, pokeweed antiviral protein, tritin, Shigella toxin, and Pseudomonas exotoxin A).

Anti-Vascular, Immunomodulatory, and/or Pharmaceutical Compositions

For administration to a subject, the compounds described herein can be provided in pharmaceutically acceptable compositions. These pharmaceutically acceptable compositions comprise a prophylatically- or therapeutically-effective amount of one or more of the compounds described above, formulated together with one or more pharmaceutically acceptable carriers (additives) and/or diluents. As described in detail below, the pharmaceutical compositions described herein can be formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes or gels; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly or intraocularly; (7) transdermally; (8) transmucosally; or (9) nasally. Additionally, compounds can be implanted into a patient or injected using a drug delivery system. See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. No. 3,773,919; and U.S. Pat. No. 35 3,270,960, the contents of which are incorporated by references in their entirety.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier” refers to a pharmaceutically-acceptable material, composition or vehicle for administration of an active agent, e.g., a mutagenic compound. Pharmaceutically acceptable carriers include any and all solvents, dispersion media, coatings, antibacterial and antifungal agents, isotonic and absorption delaying agents, and the like which are compatible with the activity of the active agent and are physiologically acceptable to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” are used interchangeably herein.

When administering parenterally a pharmaceutical composition described herein, it can be generally formulated in a unit dosage injectable form (solution, suspension, emulsion). The pharmaceutical formulations suitable for injection include sterile aqueous solutions or dispersions. The carrier can be a solvent or dispersing medium containing, for example, water, buffers (e.g., phosphate buffered saline), polyol (for example, glycerol, propylene glycol, liquid polyethylene glycol, and the like), suitable mixtures thereof. In some embodiments, the pharmaceutical carrier can be a buffered solution (e.g. PBS).

An oral composition can be prepared in any orally acceptable dosage form including, but not limited to, tablets, capsules, emulsions and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added. Liquid preparations for oral administration can also be prepared in the form of a dry powder to be reconstituted with a suitable solvent prior to use.

In some embodiments, the anti-vascular composition can be formulated in an emulsion, a paste or a gel. The anti-vascular gel composition can be implanted locally to a diseased region.

In other embodiments, the compounds or compositions can be administered in the form of sustained-release or controlled-release formulations, e.g., to reduce repeated administration and inconvenience to the patient. Many types of delivery systems are available and known to those of ordinary skill in the art. They include, for example, polymer-based systems such as polylactic and/or polyglycolic acids, poly(lactic-co-glycolic acid), polyethylene glycol, polyanhydrides, poly(sebacic acid-co-ricinoleic acid), polycaprolactones, copolyoxalates, polyesteramides, polyorthoesters, polyhydroxybutyric acid, and/or combinations thereof. Microcapsules of some of the foregoing polymers containing drugs are described in, for example, U.S. Pat. No. 5,075,109. Accordingly, in some embodiments of the anti-vascular compositions, the compound or composition can be encapsulated in one or more polymeric vehicles such as microcapsules described above.

Other examples of controlled-release formulations include nonpolymer systems that are lipid-based including sterols such as cholesterol, cholesterol esters, and fatty acids or neukal fats such as mono-, di- and triglycerides; hydrogel release systems; liposome-based systems; phospholipid based-systems; silastic systems; peptide based systems; or partially fused implants. Specific examples include, but are not limited to, erosional systems in which the composition is contained in a form within a matrix (for example, as described in U.S. Pat. Nos. 4,452,775, 4,675,189, 5,736,152, 4,667,014, 4,748,034 and -29 5,239,660), or diffusional systems in which an active component controls the release rate (for example, as described in U.S. Pat. Nos. 3,832,253, 3,854,480, 5,133,974 and 5,407,686). The formulation can be as, for example, microspheres, hydrogels, polymeric reservoirs, cholesterol matrices, or polymeric systems. In some embodiments, the system can allow sustained or controlled release of the composition to occur, for example, through control of the diffusion or erosion/degradation rate of the formulation containing the composition. In addition, a pump-based hardware delivery system can be used to deliver one or more embodiments of the compositions described herein. Use of a long-term sustained release formulations or implants can be particularly suitable for treatment of chronic conditions, such as the suspected presence of dormant metastases for cancer treatment or chronic inflammation-associated diseases. Long-term release, as used herein, means that a formulation or an implant is made and arranged to deliver a compound described herein at a therapeutic level for at least 2 weeks, at least 1 month, at least 2 months, at least 3 months, at least 4 months, at least 5 months, at least 6 months, or longer. In some embodiments, the long-term release refers to a formulation or an implant being configured to deliver a compound at a therapeutic level over several months or longer.

In some embodiments, the composition described herein can be formulated in a form of coating for, or incorporated into a biomedical device or implant, e.g., for sustained release of the compound into the body after implantation. By way of example only, the biomedical device or implant can be a stent. A variety of stents can be utilized for the methods or compositions described herein, including for example, esophageal stents, vascular stents, biliary stents, pancreatic stents, ureteric and urethral stents, lacrimal stents, Eustachian tube stents, fallopian tube stents and tracheal/bronchial stents.

Stents can be readily obtained from commercial sources, or constructed in accordance with well-known techniques. Representative examples of stents include those described in U.S. Pat. No. 4,768,523, entitled “Hydrogel Adhesive;” U.S. Pat. No. 4,776,337, entitled “Expandable Intraluminal Graft, and Method and Apparatus for Implanting and Expandable Intraluminal Graft;” U.S. Pat. No. 5,041,126 entitled “Endovascular Stent and Delivery System;” U.S. Pat. No. 5,052,998 entitled “Indwelling Stent and Method of Use;” U.S. Pat. No. 5,064,435 entitled “Self-Expanding Prosthesis Having Stable Axial Length;” U.S. Pat. No. 5,089,606, entitled “Water-insoluble Polysaccharide Hydrogel Foam for Medical Applications;” U.S. Pat. No. 5,147,370, entitled “Nitinol Stent for Hollow Body Conduits;” U.S. Pat. No. 5,176,626, entitled “Indwelling Stent;” U.S. Pat. No. 5,213,580, entitled “Biodegradable polymeric Endoluminal Sealing Process;” and U.S. Pat. No. 5,328,471, entitled “Method and Apparatus for Treatment of Focal Disease in Hollow Tubular Organs and Other Tissue Lumens.”

In some embodiments, stents can be coated with anti-vascular compositions described herein in a variety of manners, including for example: (a) by directly affixing to the stent an anti-vascular composition (e.g., by either spraying the stent with a polymer/drug film, or by dipping the stent into a polymer/drug solution), (b) by coating the stent with a substance such as a hydrogel which will in turn absorb the anti-vascular composition, (c) by interweaving anti-vascular composition coated thread (or the polymer itself formed into a thread) into the stent structure, (d) by inserting the stent into a sleeve or mesh which is comprised of or coated with an anti-vascular composition, or (e) constructing the stent itself with an anti-vascular composition. In certain embodiments of vascular stents, the anti-vascular composition can be also anti-thrombogenic (e.g., inhibiting formation of blood clots).

In some embodiments, targeted delivery of the compositions described herein is desirable to reduce the potential side effects of the compositions on normal healthy cells or tissues. In such embodiments, the addition of target moiety to the surface of delivery vehicles, devices or implants such as microspheres, microcapsules, millirods or stents can specifically bring the compound as described herein to the target diseased cell. The particular cell surface targets that are chosen for the target moiety will depend upon the target cell. Cells can be specifically targeted, for example, by the use of antibodies against unique proteins, lipids or carbohydrates that are present on the cell surface. A skilled artisan can readily determine such molecules based on the general knowledge in the art. For example, certain tumors frequently possess a large amount of a particular cell surface receptor (e.g. neu with breast cancers), or an abnormal form of a particular protein. Therefore, a tumor antigen can serve as a specific target for delivering the compositions or formulations described herein into the tumor cells, to inhibit growth and/or proliferation of the tumor cells or to destroy the tumor cells. Any known tumor antigen expressed on the tumor cell surface can be used for generating an antibody to serve as a target moiety.

Transdermal patches can also be used to provide controlled delivery of the formulations and compositions as disclosed herein to specific regions of the body. Such dosage forms can be made by dissolving or dispensing the component in the proper medium. Absorption enhancers can also be used to increase the flux of the compound across the skin. The rate can be controlled by either providing a rate-controlling membrane or by dispersing the compound in a polymer matrix or gel. Such transdermal patches are useful for treating parts of the body where abnormally stimulated neovascularization occurs, such as inflammatory diseases, for example rheumatism and psoriasis among others, diabetic retinopathy and cancer, for example skin cancer or other skin related neovascular conditions (such as psoriasis) or malignancies.

Anti-vascular or pharmaceutical compositions can be formulated for topical or intraocular administration to the eye, for example as a treatment for retinopathy, such as diabetic retinopathy or retinopathy of prematurity (ROP) or for the treatment of immune-mediated conditions of the eye such as autoimmune diseases, allergic or inflammatory conditions, and corneal transplants. The compound described herein (e.g., dehydro-alpha-lapachone) can be delivered in a pharmaceutically acceptable ophthalmic vehicle, such that the component is maintained in contact with the ocular surface for a sufficient time period to allow the component to penetrate the corneal and internal regions of the eye, as for example the anterior chamber, posterior chamber, vitreous body, aqueous humor, vitreous humor, cornea, iris/cilary, lens, choroid/retina and sclera. The pharmaceutically acceptable ophthalmic vehicle can, for example, be an ointment or an encapsulating material.

Composition or formulation as disclosed herein capable of preventing or treating diabetic retinopathy can be tested by in vitro studies of endothelial cell proliferation and in other models of diabetic retinopathy, such as Streptozotocin. In addition, color Doppler imaging can be used to evaluate the action of a drug in ocular pathology (Valli et al., Ophthalmologica 209(13): 115-121 (1995)). Color Doppler imaging is a recent advance in ultrasonography, allowing simultaneous two-dimension imaging of structures and the evaluation of blood flow. Accordingly, retinopathy can be analyzed using such technology. The manner in which the compositions and formulations as disclosed herein are administered is dependent, in part, upon whether the treatment of a disease associated with vascular hyperpermeability, including non-proliferative retinopathy is prophylactic or therapeutic. For example, the manner in which compositions and formulations as disclosed herein are administered for treatment of retinopathy is dependent, in part, upon the cause of the retinopathy. Specifically, given that diabetes is the leading cause of retinopathy, the compositions and formulations as disclosed herein can be administered preventatively as soon as the pre-diabetic retinopathy state is detected.

In one embodiment, the delivery is by intranasal administration of the composition, especially for use in therapy of the brain and related organs (e.g., meninges and spinal cord). Along these lines, intraocular administration is also possible. Suitable formulations can be found in Remington's Pharmaceutical Sciences, 16th and 18th Eds., Mack Publishing, Easton, Pa. (1980 and 1990), and Introduction to Pharmaceutical Dosage Forms, 4th Edition, Lea & Febiger, Philadelphia (1985), each of which is incorporated herein by reference.

The concentration or content of the compound described herein in the anti-vascular or pharmaceutical composition can be appropriately selected according to the physicochemical properties of the composition. When the composition is in a liquid form, the concentration can be about 0.0005 to about 30% (w/v) and preferably about 0.005 to about 25% (w/v). When the composition is a solid, the content can be about 0.01 to about 90% (w/w) and preferably about 0.1 to about 50% (w/w).

As described earlier, determination of a prophylatically or therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices, are preferred.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the IC50 (i.e., the concentration of the therapeutic which achieves a half-maximal inhibition of symptoms) as determined in cell culture. Levels in plasma may be measured, for example, by high performance liquid chromatography. The effects of any particular dosage can be monitored by a suitable bioassay.

In some embodiments, the anti-vascular or pharmaceutical compositions described herein can further comprise at least one active agent, which will be discussed in detail later. Examples of such active agents can include, but are not limited to, therapeutic agents, anti-cancer agents, anti-angiogenic or anti-vascular agents, anti-inflammation agents, VEGF inhibitors (e.g., AVASTIN®), antibiotics, anti-viral agents, anti-fungal agents, anti-protozoal agents, hormones, radioactive-agents, toxins, anesthetics, and any combinations thereof.

If necessary, additives such as a preservative (e.g. benzyl alcohol, ethyl alcohol, benzalkonium chloride, phenol, chlorobutanol, etc.), an antioxidant (e.g. butylhydroxyanisole, propyl gallate, ascorbyl palmitate, alpha-tocopherol, etc.), and a thickener (e.g. lecithin, hydroxypropylcellulose, aluminum stearate, etc.) can be used in the compositions and formulations as disclosed herein.

In addition to the above-mentioned components, a stabilizer for further improving the stability of the compositions and formulations as disclosed herein, such as an antioxidant or a chelating agent, an isotonizing agent for adjusting the osmolarity, an auxiliary emulsifier for improving the emulsifying power, and/or an emulsion stabilizer for improving the stability of the emulsifying agent can be incorporated. The isotonizing agent that can be used includes, for example, glycerin, sugar alcohols, monosaccharides, disaccharides, amino acids, dextran, albumin, etc. These isotonizing agents can be used individually or in combination, with two or more. An emulsion stabilizer that can be used, which includes cholesterol, cholesterol esters, tocopherol, albumin, fatty acid amide derivatives, polysaccharides, polysaccharide fatty acid ester derivatives, etc.

The compositions and formulations as disclosed herein can further comprise a viscogenic substance which can adhere to the digestive tract mucosa due to its viscosity expressed on exposure to water. The examples of the viscogenic substance include, but are not particularly limited as long as it is pharmaceutically acceptable, such as polymers (e.g. polymers or copolymers of acrylic acids and their salts) and natural-occurring viscogenic substances (e.g. mucins, agar, gelatin, pectin, carrageenin, sodium alginate, locust bean gum, xanthan gum, tragacanth gum, arabic gum, chitosan, pullulan, waxy starch, sucralfate, curdlan, cellulose, and their derivatives). Furthermore, for controlling the release of the active drug or for formulation purposes, the additives conventionally used for preparing the oral compositions can be added. Example of the additives include excipients (e.g. lactose, corn starch, talc, crystalline cellulose, sugar powder, magnesium stearate, mannitol, light anhydrous silicic acid, magnesium carbonate, calcium carbonate, L-cysteine, etc.), binders (e.g. starch, sucrose, gelatin, arabic gum powder, methylcellulose, carboxymethylcellulose, carboxymethylcellulose sodium, hydroxypropylcellulose, hydroxypropylmethylcellulose, polyvinylpyrrolidone, pullulan, dextrin, etc.), disintegrators (e.g. carboxymethylcellulose calcium, low-substituted hydroxypropylcellulose, croscarmellose sodium, etc.), anionic surfactants (e.g. sodium alkylsulfates etc.), nonionic surfactants (e.g. polyoxyethylene sorbitan fatty acid esters, polyoxyethylene fatty acid esters, polyoxyethylene-castor oil derivatives, etc.), antacids and mucous membrane protectants (e.g. magnesium hydroxide, magnesium oxide, aluminum hydroxide, aluminum sulfate, magnesium metasilicate aluminate, magnesium silicate aluminate, sucralfate, etc.), cyclodextrin and the corresponding carboxylic acid (e.g. maltosyl-beta-cyclodextrin, maltosyl-beta-cyclodextrin-carboxylic acid, etc.), colorants, corrigents, adsorbents, antiseptics, moistening agents, antistatic agents, disintegration retardants, and so on. The proportion of these additives can be appropriately selected from the range that can keep the stability and absorption of the basis.

The formulations and pharmaceutical composition can, if desired, be presented in a suitable container (e.g., a pack or dispenser device), such as an FDA approved kit, which can contain one or more unit dosage forms containing the carrier portion containing the targeting and immune response triggering portions.

### Conditions Involving Abnormal Vasculatures

The anti-vascular compositions as disclosed herein can be used to treat or prevent a condition in which abnormal vasculatures or angiogenesis is involved or required for the pathology of the condition. The term ““abnormal vasculatures” refers to at least one blood vessel having at least one abnormal attribute when compared to a plurality of different blood vessel attributes of healthy subjects. Exemplary abnormal attributes of blood vessels include, but are not limited to, hyperpermeability, tortuosity, abnormal vascular density, abnormal blood vessel diameter, and any combinations thereof. In some embodiments, such condition includes vascular proliferative diseases or disorders. As used herein, the term “proliferative diseases” refers to the development of cells that result in unwanted or undesirable physiological consequences, such as with a tumor or inflammation or hyperpermeable, abnormal vasculature, viral infections, bacterial infections and fungal infections. Angiogenesis should be understood to be a proliferative process. Thus, in certain embodiments, the term “proliferation” can apply to the development of blood vessels. Such development is also referred to herein as “angiogenesis.” In some instances, the term “angiogenesis”, as used herein refers to the sprouting of new blood vessels from pre-existing blood vessels, characterized by endothelial cell proliferation and migration triggered by certain pathological conditions, such as the growth of tumors, metastasis, AMD and arthritis, among others. Accordingly, in some embodiments, such condition includes angiogenic diseases or disorders. In some embodiments, such condition includes vascular diseases or disorders. In some embodiments, such conditions include inflammatory diseases.

As used herein, the term “angiogenic- or vascular-associated diseases” refers to diseases, disorders or conditions that are dependent on a rich blood supply and blood vessel proliferation for the disease pathological progression (e.g. metastatic tumors) or diseases or disorders that are the direct result of aberrant blood vessel proliferation or blood vessel hyperpermeability (e.g. diabetic retinopathy and hemangiomas). Non-limiting examples of “conditions involving abnormal vasculatures” or “angiogenic- or vascular-associated diseases” include, but are not limited to, tuberculosis, autoimmune diseases, wound repair, hypertrophic scar or keloid, neointimal hyperplasia, psoriasis, age-related macular degeneration (AMD), thyroid hyperplasia, preeclampsia, hemangiomas, rheumatoid arthritis and osteoarthritis, Alzheimer's disease, obesity, pleural effusion, atherosclerosis, endometriosis, diabetic/other retinopathies, ocular neovascularizations, edemas, chronic obstructive pulmonary diseases, asthma, cystic fibrosis, transplant rejection, allergic reaction, multiple sclerosis, epithelial infection, and conditions involving or characterized by vascular hyperpermeability, inflammation, angiogenesis or vascular hyperproliferation. In some embodiments, the “conditions involving abnormal vasculatures” or “angiogenic- or vascular-associated diseases” can include cancer or metastatic tumors. In some embodiments, the “conditions involving abnormal vasculatures” or “angiogenic- or vascular-associated diseases” exclude cancer or metastatic tumors.

In one embodiment, a condition to be treated or prevented by the compositions and methods described herein is cancer, where the cells are rapidly-dividing neoplastic cancer cells, and where the neoplastic cells require an efficient blood supply to maintain continued growth of the tumor. As used herein, cancer generally refers to any of various non-metastatic cancers, and malignant neoplasms (metastatic cancers) characterized by proliferation of anaplastic cells that tend to invade surrounding tissue and metastasize to new body sites. In some embodiments, cancer also refers to the pathological condition characterized by such malignant neoplastic growths. The blood vessels provide conduits to metastasize and spread elsewhere in the body. Upon arrival at the metastatic site, the cancer cells then work on establishing a new blood supply network. Administration of a composition comprising at least one compound described herein (e.g., dehydro-alpha-lapachone) can be used to inhibit proliferation of primary tumors, and/or to inhibit its metastasis to secondary sites, e.g., by pruning tumor vasculatures.

Examples of cancer include, but are not limited to, any solid tumors, papilloma/carcinoma, choriocarcinoma, endodermal sinus tumor, teratoma, adenoma/adenocarcinoma, melanoma, fibroma, lipoma, leiomyoma, rhabdomyoma, mesothelioma, angioma, osteoma, chondroma, glioma, lymphoma/leukemia, squamous cell carcinoma, small cell carcinoma, large cell undifferentiated carcinomas, basal cell carcinoma and sinonasal undifferentiated carcinoma. The types of sarcomas include soft tissue sarcoma such as alveolar soft part sarcoma, angiosarcoma, dermatofibrosarcoma, desmoid tumor, desmoplastic small round cell tumor, extraskeletal chondrosarcoma, extraskeletal osteosarcoma, fibrosarcoma, hemangiopericytoma, hemangiosarcoma, Kaposi's sarcoma, leiomyosarcoma, liposarcoma, lymphangiosarcoma, lymphosarcoma, malignant fibrous histiocytoma, neurofibrosarcoma, rhabdomyosarcoma, synovial sarcoma, and Askin's tumor, Ewing's sarcoma (primitive neuroectodermal tumor), malignant hemangioendothelioma, malignant schwannoma, osteosarcoma, and chondrosarcoma. Abnormal build up and growth of blood vessels in the skin or internal organs in the form of hemangiomas can also be treated according to the methods and/or compositions described herein.

In one embodiment, a condition to be treated or prevented by the methods and compositions described herein includes triple-negative breast cancer, which is a breast cancer subtype negative in estrogen receptor, progesterone receptor, and C-erbB-2. Triple-negative breast cancer cells generally exhibit phenotypes that allow them to escape effective immune surveillance and response through immunosuppressive pathways. For example, patients with triple-negative cancer have increase in tryptophan catabolism through elevation of the enzyme IDO, which has immunosuppressive characteristics. As shown in Examples, DAL can repress the Rho-GTPase Rac1, which can increase cancer cell motility through remodeling of the actin cytoskeleton, and can allosterically inhibit indoleamine-2,3-dioxygenase (IDO). Accordingly, inhibition or reduction of cell motility and/or activity of IDO using the methods and compositions described herein can treat or prevent such disease.

In one embodiment, a condition to be treated or prevented by the methods and compositions described herein is age-related macular degeneration. It is known that VEGF contributes to abnormal blood vessel growth from the choroid layer of the eye into the retina, similar to what occurs during the wet or neovascular form of age-related macular degeneration. Macular degeneration, often called AMD or ARMD (age-related macular degeneration), is the leading cause of vision loss and blindness in individuals aged 65 and older. In order for new blood vessels to grow (neovascularization) beneath the retina and leak blood and fluid, endothelial cells must proliferate. This proliferation is uneven, and results in the leakage described above, which causes permanent damage to light-sensitive retinal cells, which die off and create blind spots in central vision or the macula. Accordingly, inhibition or reduction of neovascularization in an eye using the methods and compositions described herein can treat or prevent such disease.

In one embodiment, a condition to be treated or prevented using the methods and compositions described herein is angiogenesis-dependent retinopathy-abnormal blood vessel growth associated with eye diseases. In diabetic retinopathy (i.e. diabetic eye diseases) and retinopathy of prematurity (ROP affecting prematurely born babies), VEGF is released, promoting blood vessel formation—thus, such retinopathies are angiogenic diseases or disorders. Released by the retina (light-sensitive nerve tissue at the back of the eye) when normal blood vessels are damaged by tiny blood clots in diabetes, VEGF turns on its receptor and ignites a chain reaction that culminates in new blood vessel growth. However, due to rapid and/or uneven proliferation, the newly-formed blood vessels are faulty, and they leak, bleed and promote formation of scar tissue that can cause retinal detachment and result in severe vision loss. Previous studies have shown that the angiogenesis in diabetic retinopathies can be treated via growth factor (e.g., vascular endothelial growth factor (VEGF), insulin like growth factor-1) blockade, integrin (e.g., alpha-v beta-3) blockade or extracellular matrix alteration (e.g., with steroid compounds), or interference with intracellular signal transduction pathways (e.g., PKC and mitogen activated protein kinase pathway proteins). In some embodiments, anti-angiogenic treatment for such angiogenesis-dependent retinopathies can employ the compositions and the methods described herein. In one embodiment, the subject in need of treatment can be a mammal, such as a human, and a domestic pet, e.g., a dog or a cat.

In one embodiment, a condition to be treated or prevented using the methods and compositions described herein is rheumatoid arthritis. Rheumatoid arthritis (RA) is characterized by synovial tissue swelling, leukocyte ingress and new blood vessel growth from existing vessels. The expansion of the synovial lining of joints in rheumatoid arthritis (RA) and the subsequent invasion by the pannus of underlying cartilage and bone necessitate an increase in the vascular supply to the synovium, in order to cope with the increased requirement for oxygen and nutrients. Thus, RA is an angiogenesis-mediated disease or disorder. Even in early RA, some of the earliest histological observations indicate formation of blood vessels. A mononuclear infiltrate characterizes the synovial tissue along with a luxuriant vasculature. Endothelial cell proliferation is integral to formation and/or maintenance of the inflammatory pannus and without it, leukocyte ingress could not occur (Paleolog, E. M., 2002; Koch, A. E., 2000). Disruption of the formation of new blood vessels would not only prevent delivery of nutrients to the inflammatory site, it could also reduce joint swelling due to the additional activity of VEGF, a potent proangiogenic and vascular permeability factor in RA.

In one embodiment, a condition to be treated or prevented by the methods and compositions described herein is Alzheimer's disease. Alzheimer's disease (AD) is the most common cause of dementia worldwide. AD is characterized by an excessive cerebral amyloid deposit leading to degeneration of neurons and eventually to dementia. The exact cause of AD is still unknown. It has been shown by epidemiological studies that long-term use of non-steroidal anti-inflammatory drugs, statins, histamine H2-receptor blockers, or calcium-channel blockers, all of which are cardiovascular drugs with an anti-proliferative effect, can be used to prevent Alzheimer's disease and/or influence the outcome of AD patients. Therefore, it has been speculated that endothelial cell proliferation in the brain vasculature can play an important role in AD, i.e., AD can be an angiogenesis-related or -mediated disease. In Alzheimer's disease, the brain endothelium secretes the precursor substrate for the beta-amyloid plaque and a neurotoxic peptide that selectively kills cortical neurons. Moreover, amyloid deposit in the vasculature leads to endothelial cell apoptosis and endothelial cell activation, which leads to neovascularization. Previous studies have shown that vessel formation can be blocked by the VEGF antagonist SU 4312 as well as by statins, indicating that anti-proliferative or anti-angiogenic strategies can interfere with endothelial cell activation in AD (Schultheis s C., el. al., 2006; Grammas P., et. al., 1999) and can be used for preventing and/or treating AD.

In one embodiment, a condition to be treated or prevented by the methods and compositions described herein is obesity. It has been shown that reduced vascularity in the adipose tissue can inhibit the rate of growth of the adipose tissue and obesity development (Ebba Bråakenhielm et. al., Circulation Research, 2004; 94:1579). As such, obesity can be an angiogenesis-related or -mediated disease or disorder.

In one embodiment, a condition to be treated or prevented by the methods and compositions described herein is endometriosis. Excessive endometrial angiogenesis is believed to be an important mechanism in the pathogenesis of endometriosis (Healy, D L., et. al., 1998). The endometrium of patients with endometriosis shows enhanced endothelial cell proliferation. Moreover, there is an elevated expression of the cell adhesion molecule integrin vβ3 in more blood vessels in the endometrium of women with endometriosis when compared with normal women. Strategies that inhibit endothelial cell proliferation and/or cell adhesion can be used to treat endometriosis. In some embodiments, the compound described herein can reduce adhesion of endothelial cells, thus providing a method to treat or prevent endometriosis.

In some embodiments, a condition to be treated or prevented by the methods and compositions described herein is hypertrophic scars and/or keloids. Briefly, healing of wounds and scar formation occurs in three phases: inflammation, proliferation, and maturation. The first phase, inflammation, occurs in response to an injury which is severe enough to break the skin. During this phase, which lasts 3 to 4 days, blood and tissue fluid form an adhesive coagulum and fibrinous network which serves to bind the wound surfaces together. This is then followed by a proliferative phase in which there is ingrowth of capillaries and connective tissue from the wound edges, and closure of the skin defect. Finally, once capillary and fibroblastic proliferation has ceased, the maturation process begins wherein the scar contracts and becomes less cellular, less vascular, and appears flat and white. This final phase can take between 6 and 12 months.

If too much connective tissue is produced and the wound remains persistently cellular, the scar can become red and raised. If the scar remains within the boundaries of the original wound it is a hypertrophic scar, but if it extends beyond the original scar and into the surrounding tissue, the lesion is a keloid. Hypertrophic scars and keloids are generally produced during the second and third phases of scar formation. Several wounds are particularly prone to excessive endothelial and fibroblastic proliferation, including burns, open wounds, and infected wounds. With hypertrophic scars, some degree of maturation occurs and gradual improvement occurs. In the case of keloids however, an actual tumor is produced which can become quite large. Spontaneous improvement in such cases rarely occurs.

Therefore, within one embodiment described herein, either anti-vascular compound alone, or anti-vascular compositions as described above can be directly injected into a hypertrophic scar or keloid, in order to prevent the progression of these lesions. The frequency of injections will depend upon the administration methods and/or routes, and the clinical response. This therapy can be used in the prophylactic treatment of conditions which are known to result in the development of hypertrophic scars and keloids (e.g., burns). In some embodiments, the therapy comprising administering the compound or the composition described herein can be initiated after the proliferative phase has initiated (approximately 14 days after the initial injury), but before hypertrophic scar or keloid development.

In one embodiment, conditions or disorders to be treated or prevented by the methods and compositions described herein can be vascular diseases, e.g., atherosclerosis. Previous studies have shown that neovascularization of the vessel wall can play a fundamental role in the pathophysiology of atherosclerosis. Angiogenesis within atherosclerotic plaques can cause plaque progression as immature blood vessels leak red blood cells and inflammatory mediators into the plaque center. Accumulation of free cholesterol from red blood cell membranes can potentially increases the size of the necrotic core and triggers a chain of events that promote plaque destabilization. Jain R. K. et la., 4 Nat Clin Pract Cardiovasc Med. 491 (2007). Accordingly, in some embodiments, anti-vascular compositions described herein, e.g., vascular stents described herein comprising the anti-vascular compound (e.g., dehydro-alpha-lapachone) or anti-vascular composition described herein, can be used to stabilize vulnerable ‘rupture-prone’ plaques, e.g., by pruning and normalizing immature intraplaque vessels, preventing further intraplaque hemorrhage. Thus, the methods for anti-angiogenic or anti-vascular therapy described herein can limit necrotic core enlargement, further luminal narrowing and the degree of inflammation.

Similarly, anti-vascular compositions can also be used for treating or preventing acute myocardial infarction, and stroke, because neovascularization and/or vascular hyperproliferation or hyperpermeability can directly or indirectly induce such diseases or disorders. For example, acute myocardial infarction is generally triggered by a disruption of an atherosclerotic plaque in an epicardial coronary artery, which leads to a clotting cascade, sometimes resulting in total occlusion of the artery.

In some embodiments, the compositions and methods as disclosed herein can decrease adhesion and/or motility of endothelial cells responsible for blood vessel formation and/or maintenance, thus inhibiting angiogenesis or changing vasculature organization. Not to be bound by theory, such decrease in adhesion and/or motility of endothelial cells is mediated by a decrease in activity and/or expression of Rac1 including its regulators. Accordingly, the compositions and methods as disclosed herein can be used for treating or preventing any diseases or disorders known to be induced, mediated or caused by abnormal vasculatures, excessive angiogenesis, endothelial dysfunction or any combinations thereof, for example, but not limited to, cancer, inflammatory diseases, diabetic retinopathy, rheumatoid arthritis, psoriasis and other angiogenic- or vascular-associated diseases.

Exemplary Embodiments of the Methods and Compositions for Treating or Preventing a Condition Involving Abnormal Vasculatures, or an Anti-Angiogenic or Anti-Vascular Disease or Disorder can be Also Described by any One of the Following Numbered Paragraphs:
1. A method of treating or preventing a condition involving abnormal vasculatures, the method comprising administering to a subject a composition comprising a compound of formula (I) or an analog, derivative, isomer, prodrug, or pharmaceutically acceptable salt thereof, wherein the compound of formula (I) has anti-vascular activity with a structure:

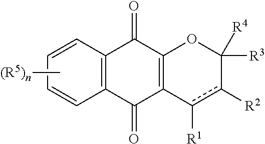

wherein:
R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)O R⁶, C(O)R⁶, C(O)OR⁶, SR⁶, or SO₂R⁶, each of which can be optionally substituted;
R³ and R⁴ are each independently selected from hydrogen, alkyl, alkenyl, cyclyl, heterocyclyl, aryl, heteroaryl, each of which can be optionally substituted;
R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R⁶ is independently selected for each occurrence, hydrogen, alkyl, alkenyl, alkynyl, cyclyl, heterocyclyl, aryl, or heteroaryl, each of which can be optionally substituted;
the dotted line between the carbons to which R¹ and R² are bonded represents an optional double bond; and
n is 0, 1, 2, 3, or 4.
2. The method of paragraph 1, wherein the compound of formula (I) is administered in a combination therapy.
3. The method of paragraph 2, wherein the combination therapy comprises administering to the subject at least one active agent.
4. The method of paragraph 3, wherein the at least one active agent is selected from the group consisting of therapeutic agents, anti-cancer agents, anti-angiogenic or anti-vascular agents, anti-inflammation agents, VEGF inhibitors, antibiotics, anti-viral agents, anti-fungal agents, anti-protozoal agents, hormones, radioactive agents, toxins, anesthetics, and any combinations thereof.
5. The method of any of paragraphs 1-4, wherein the compound of formula (I) has anti-tumor activity.
6. The method of any of paragraphs 1-5, wherein the compound of formula (I) decreases immune tolerance to said condition.
7. The method of any of paragraphs 1-6, wherein the compound of formula (I) is a product from Tabebuia Avellaneda tree.
8. The method of any of paragraphs 1-7, wherein the compound of formula (I) is dehydro-alpha-lapachone or an analog, derivative, isomer, prodrug, or pharmaceutically acceptable salt thereof.
9. The method of any of paragraphs 1-8, wherein the composition is administered in pulses.
10. The method of any of paragraphs 1-8, wherein the composition is administered by sustained release.
11. The method of any of paragraphs 1-10, wherein the composition is administered to the subject over a term of at least about 2 months.
12. The method of any of paragraphs 1-11, wherein an effective amount of the composition or the compound administered to the subject is from 0.001 mg/kg to about 500 mg/kg.
13. The method of paragraph 12, wherein the effective amount of the composition or the compound is from about 1 mg/kg to about 100 mg/kg.
14. The method of paragraph 13, wherein the effective amount of the composition or the compound is from about 10 mg/kg to about 50 mg/kg.
15. The method of any of paragraphs 1-14, wherein the condition is selected from the group consisting of tuberculosis, autoimmune diseases, wound repair, hypertrophic scar or keloid, neointimal hyperplasia, psoriasis, macular degeneration, age-related macular degeneration (AMD), thyroid hyperplasia, preeclampsia, hemangiomas, rheumatoid arthritis and osteoarthritis, Alzheimer's disease, obesity, pleural effusion, atherosclerosis, endometriosis, diabetic retinopathies, other retinopathies, ocular neovascularizations, edemas, chronic obstructive pulmonary diseases, asthma, cystic fibrosis, transplant rejection, allergic reaction, multiple sclerosis, epithelial infection, and conditions involving or characterized by vascular hyperpermeability, inflammation, angiogenesis or vascular hyperproliferation.
16. The method of any of paragraphs 1-14, wherein the condition is cancer.
17. The method of paragraph 16, wherein the cancer is a metastatic tumor.
18. The method of paragraph 17, wherein the metastatic tumor is a metastatic breast tumor.
19. The method of paragraph 18, wherein the metastatic breast tumor is triple-negative breast tumor.
20. The method of any of paragraphs 1-19, wherein the condition involves abnormal vasculatures in an eye of the subject.
21. The method of paragraph 20, wherein the condition involving abnormal vasculatures in the eye of the subject is macular degeneration.
22. An anti-vascular composition comprising an effective amount of a compound of formula (I) or an analog, derivative, isomer, prodrug, or pharmaceutically acceptable salt thereof, wherein said composition is formulated for a controlled release for at least about 2 months in a subject and has anti-vascular activity with a structure:

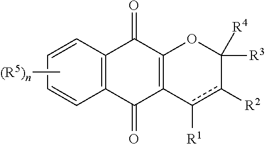

wherein:
R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)O R⁶, C(O)R⁶, C(O)OR⁶, SR⁶, or SO₂R⁶, each of which can be optionally substituted;
R³ and R⁴ are each independently selected from hydrogen, alkyl, alkenyl, cyclyl, heterocyclyl, aryl, heteroaryl, each of which can be optionally substituted;
R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R⁶ is independently selected for each occurrence, hydrogen, alkyl, alkenyl, alkynyl, cyclyl, heterocyclyl, aryl, or heteroaryl, each of which can be optionally substituted;
the dotted line between the carbons to which R¹ and R² are bonded represents an optional double bond; and
n is 0, 1, 2, 3, or 4.
23. The anti-vascular composition of paragraph 22, wherein the compound of formula (I) is further conjugated to a macromolecule.
24. The anti-vascular composition of paragraph 22 or 23, wherein the compound is encapsulated in one or more polymeric vehicles.
25. The anti-vascular composition of paragraph 24, where said one or more polymeric vehicles comprise a polymer selected from the group consisting of polylactic and/or polyglycolic acids, poly(lactic-co-glycolic acid), polyethylene glycol, polyanhydrides, poly(sebacic acid-co-ricinoleic acid), polycaprolactones, copolyoxalates, polyesteramides, polyorthoesters, polyhydroxybutyric acid, and any combinations thereof.
26. The anti-vascular composition of any of paragraphs 22-25, wherein the compound of formula (I) has anti-tumor activity.
27. The anti-vascular composition of any of paragraphs 22-26, wherein the compound of formula (I) decreases immune tolerance to a condition being treated.
28. The anti-vascular composition of any of paragraphs 22-27, wherein the compound of formula (I) is a product from Tabebuia Avellaneda tree.
29. The anti-vascular composition of any of paragraphs 22-28, wherein the compound of formula (I) is dehydro-alpha-lapachone, or an analog, derivative, isomer, prodrug, or pharmaceutically acceptable salt thereof.
30. The anti-vascular composition of any of paragraphs 22-29, further comprising at least one active agent.
31. The anti-vascular composition of paragraph 30, wherein said at least one active agent is selected from the group consisting of therapeutic agents, anti-cancer agents, anti-angiogenic or anti-vascular agents, anti-inflammation agents, VEGF inhibitors, antibiotics, anti-viral agents, anti-fugal agents, anti-protozoal agents, hormones, radioactive agents, toxins, anesthetics, and any combinations thereof.
32. The anti-vascular composition of any of paragraphs 22-31, wherein the effective amount is about 0.001 mg/kg to about 500 mg/kg.
33. The anti-vascular composition of any of paragraphs 22-31, wherein the effective amount is about 1 mg/kg to about 100 mg/kg.
34. The anti-vascular composition of any of paragraphs 22-31, wherein the effective amount is about 10 mg/kg to about 50 mg/kg.
35. A method of treating or preventing a condition involving abnormal vasculatures, the method comprising administering to a subject a composition comprising a compound of formula (I) or an analog, derivative, isomer, prodrug, or pharmaceutically acceptable salt thereof, wherein the condition involving abnormal vasculatures excludes cancer or metastatic tumors, and wherein the compound of formula (I) has anti-vascular activity with a structure:

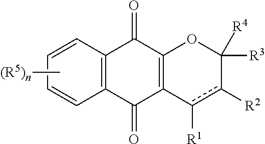

wherein:
R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)O R⁶, C(O)R⁶, C(O)OR⁶, SR⁶, or SO₂R⁶, each of which can be optionally substituted;
R³ and R⁴ are each independently selected from hydrogen, alkyl, alkenyl, cyclyl, heterocyclyl, aryl, heteroaryl, each of which can be optionally substituted;
R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R⁶ is independently selected for each occurrence, hydrogen, alkyl, alkenyl, alkynyl, cyclyl, heterocyclyl, aryl, or heteroaryl, each of which can be optionally substituted;
the dotted line between the carbons to which R¹ and R² are bonded represents an optional double bond; and
n is 0, 1, 2, 3, or 4.
36. The method of paragraph 35, wherein the compound of formula (I) is administered in a combination therapy.
37. The method of paragraph 36, wherein the combination therapy comprises administering to the subject at least one active agent.
38. The method of paragraph 37, wherein the at least one active agent is selected from the group consisting of therapeutic agents, anti-angiogenic or anti-vascular agents, anti-inflammation agents, VEGF inhibitors, antibiotics, anti-viral agents, anti-fungal agents, anti-protozoal agents, hormones, radioactive agents, toxins, anesthetics, and any combinations thereof.
39. The method of any of paragraphs 35-38, wherein the compound of formula (I) decreases immune tolerance to said condition.
40. The method of any of paragraphs 35-39, wherein the compound of formula (I) is a product from Tabebuia Avellaneda tree.
41. The method of any of paragraphs 35-40, wherein the compound of formula (I) is dehydro-alpha-lapachone or an analog, derivative, isomer, prodrug, or pharmaceutically acceptable salt thereof.
42. The method of any of paragraphs 35-41, wherein the composition is administered in pulses.
43. The method of any of paragraphs 35-41, wherein the composition is administered by sustained release.
44. The method of any of paragraphs 35-43, wherein the composition is administered to the subject over a term of at least about 2 months.
45. The method of any of paragraphs 35-44, wherein an effective amount of the composition or the compound administered to the subject is from 0.001 mg/kg to about 500 mg/kg.
46. The method of any of paragraphs 35-44, wherein the effective amount of the composition or the compound is from about 1 mg/kg to about 100 mg/kg.
47. The method of any of paragraphs 35-44, wherein the effective amount of the composition or the compound is from about 10 mg/kg to about 50 mg/kg.
48. The method of any of paragraphs 35-47, wherein the condition is selected from the group consisting of tuberculosis, autoimmune diseases, wound repair, hypertrophic scar or keloid, neointimal hyperplasia, psoriasis, macular degeneration, age-related macular degeneration (AMD), thyroid hyperplasia, preeclampsia, hemangiomas, rheumatoid arthritis and osteoarthritis, Alzheimer's disease, obesity, pleural effusion, atherosclerosis, endometriosis, diabetic retinopathies, other retinopathies, ocular neovascularizations, edemas, chronic obstructive pulmonary diseases, asthma, cystic fibrosis, transplant rejection, allergic reaction, multiple sclerosis, epithelial infection, and conditions involving or characterized by vascular hyperpermeability, inflammation, angiogenesis or vascular hyperproliferation, but excluding cancer and metastatic tumors.
49. A method of inhibiting or reducing angiogenesis in a subject in need thereof comprising administering to a subject a composition comprising a compound of formula (I) or an analog, derivative, isomer, prodrug, or pharmaceutically acceptable salt thereof, wherein the compound of formula (I) has anti-vascular activity with a structure:

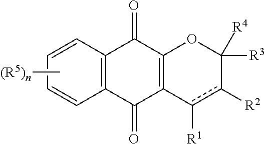

wherein:
R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)O R⁶, C(O)R⁶, C(O)OR⁶, SR⁶, or SO₂R⁶, each of which can be optionally substituted;
R³ and R⁴ are each independently selected from hydrogen, alkyl, alkenyl, cyclyl, heterocyclyl, aryl, heteroaryl, each of which can be optionally substituted;
R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R⁶ is independently selected for each occurrence, hydrogen, alkyl, alkenyl, alkynyl, cyclyl, heterocyclyl, aryl, or heteroaryl, each of which can be optionally substituted;
the dotted line between the carbons to which R¹ and R² are bonded represents an optional double bond; and
n is 0, 1, 2, 3, or 4.
50. The method of paragraph 49, wherein the subject in need thereof has an angiogenic- or vascular-associated disease or a condition involving abnormal vasculatures.
51. The method of paragraph 49, wherein the subject in need thereof is at risk of having an angiogenic- or vascular-associated disease or a condition involving abnormal vasculatures.
52. A method of reducing adhesion or motility of at least one cell comprising contacting a cell with a composition comprising a compound of formula (I) or an analog, derivative, isomer, prodrug, or pharmaceutically acceptable salt thereof, wherein the compound of formula (I) has anti-vascular activity with a structure:

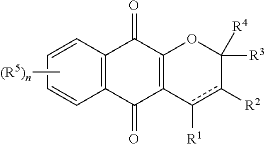

wherein:
R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)O R⁶, C(O)R⁶, C(O)OR⁶, SR⁶, or SO₂R⁶, each of which can be optionally substituted;
R³ and R⁴ are each independently selected from hydrogen, alkyl, alkenyl, cyclyl, heterocyclyl, aryl, heteroaryl, each of which can be optionally substituted;
R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R⁶ is independently selected for each occurrence, hydrogen, alkyl, alkenyl, alkynyl, cyclyl, heterocyclyl, aryl, or heteroaryl, each of which can be optionally substituted;
the dotted line between the carbons to which R¹ and R² are bonded represents an optional double bond; and
n is 0, 1, 2, 3, or 4.
53. The method of paragraph 52, wherein said at least one cell is an endothelial cell.
54. The method of paragraph 52, wherein said at least one cell is a cancer cell.
55. A method of treating or preventing triple-negative breast cancer in a subject, the method comprising administering to a subject diagnosed with or at risk of having triple-negative breast cancer a composition comprising a compound of formula (I) or an analog, derivative, isomer, prodrug, or pharmaceutically acceptable salt thereof, wherein the compound of formula (I) has anti-vascular activity with a structure:

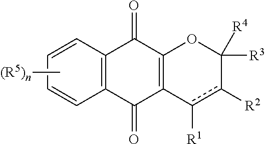

wherein:
R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)O R⁶, C(O)R⁶, C(O)OR⁶, SR⁶, or SO₂R⁶, each of which can be optionally substituted;
R³ and R⁴ are each independently selected from hydrogen, alkyl, alkenyl, cyclyl, heterocyclyl, aryl, heteroaryl, each of which can be optionally substituted;
R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R⁶ is independently selected for each occurrence, hydrogen, alkyl, alkenyl, alkynyl, cyclyl, heterocyclyl, aryl, or heteroaryl, each of which can be optionally substituted;
the dotted line between the carbons to which R¹ and R² are bonded represents an optional double bond; and
n is 0, 1, 2, 3, or 4.
56. The method of paragraph 55, wherein the compound of formula (I) is administered in a combination therapy.
57. The method of paragraph 56, wherein the combination therapy comprises administering to the subject at least one therapeutic agent.
58. The method of paragraph 57, wherein the therapeutic agent includes a chemotherapeutic agent.
59. The method of paragraph 58, wherein the chemotherapeutic agent includes paclitaxel.
60. A method of treating or preventing an ocular disease or disorder involving abnormal vasculatures in a subject, the method comprising administering to a subject diagnosed with or at risk of having an ocular disease or disorder involving abnormal vasculatures a composition comprising a compound of formula (I) or an analog, derivative, isomer, prodrug, or pharmaceutically acceptable salt thereof, wherein the compound of formula (I) has anti-vascular activity with a structure:

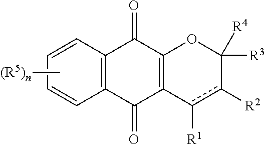

wherein:
R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)O R⁶, C(O)R⁶, C(O)OR⁶, SR⁶, or SO₂R⁶, each of which can be optionally substituted;
R³ and R⁴ are each independently selected from hydrogen, alkyl, alkenyl, cyclyl, heterocyclyl, aryl, heteroaryl, each of which can be optionally substituted;
R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R⁶ is independently selected for each occurrence, hydrogen, alkyl, alkenyl, alkynyl, cyclyl, heterocyclyl, aryl, or heteroaryl, each of which can be optionally substituted;
the dotted line between the carbons to which R¹ and R² are bonded represents an optional double bond; and
n is 0, 1, 2, 3, or 4.
61. The method of paragraph 60, wherein the ocular disease or disorder involving abnormal vasculatures is selected from a group consisting of age-related macular degeneration (AMD), proliferative diabetic retinopathy (PDR), diabetic macular edema (DME), neovascular glaucoma, corneal neovascularization (trachoma), and pterygium.
62. The method of paragraph 60 or 61, wherein the compound of formula (I) is administered in combination with an antiangiogenic therapy for an ophthalmic use.
63. The method of paragraph 62, wherein the antiangiogenic therapy for the ophthalmic use includes an anti-VEGF aptamer (pegaptanib, MACUGEN®); a Fab fragment of a monoclonal antibody directed against VEGF-A (ranibizumab, LUCENTIS® or bevacizumab, AVASTIN®); or a combination thereof.
64. A method of increasing activity of an immune system in a subject, the method comprising administering to a subject a composition comprising a compound of formula (I) or an analog, derivative, isomer, prodrug, or pharmaceutically acceptable salt thereof, wherein the compound of formula (I) inhibits or reduces activity of an immunomodulatory enzyme, and wherein the compound of formula (I) has a structure:

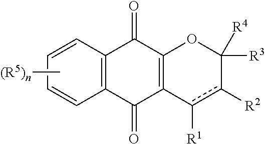

wherein:
R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)O R⁶, C(O)R⁶, C(O)OR⁶, SR⁶, or SO₂R⁶, each of which can be optionally substituted;
R³ and R⁴ are each independently selected from hydrogen, alkyl, alkenyl, cyclyl, heterocyclyl, aryl, heteroaryl, each of which can be optionally substituted;
R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R⁶ is independently selected for each occurrence, hydrogen, alkyl, alkenyl, alkynyl, cyclyl, heterocyclyl, aryl, or heteroaryl, each of which can be optionally substituted;
the dotted line between the carbons to which R¹ and R² are bonded represents an optional double bond; and
n is 0, 1, 2, 3, or 4.
65. The method of paragraph 64, wherein the immunomodulatory enzyme is indoleamine-pyrrole 2,3-dioxygenase (IDO).
66. The method of paragraph 64 or 65, wherein increasing the activity of the immune system produces an anti-tumor effect.
67. An immunomodulatory composition comprising an effective amount of a compound of formula (I) or an analog, derivative, isomer, prodrug, or pharmaceutically acceptable salt thereof, wherein the compound of formula (I) inhibits or reduces activity of an immunomodulatory enzyme, and wherein the compound of formula (I) has a structure:

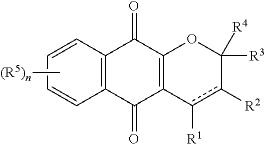

wherein:
R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)O R⁶, C(O)R⁶, C(O)OR⁶, SR⁶, or SO₂R⁶, each of which can be optionally substituted;
R³ and R⁴ are each independently selected from hydrogen, alkyl, alkenyl, cyclyl, heterocyclyl, aryl, heteroaryl, each of which can be optionally substituted;
R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R⁶ is independently selected for each occurrence, hydrogen, alkyl, alkenyl, alkynyl, cyclyl, heterocyclyl, aryl, or heteroaryl, each of which can be optionally substituted;
the dotted line between the carbons to which R¹ and R² are bonded represents an optional double bond; and
n is 0, 1, 2, 3, or 4.
68. The immunomodulatory composition of paragraph 67, wherein the effective amount is sufficient to inhibit or reduce activity of indoleamine-pyrrole 2,3-dioxygenase (IDO) by at least about 10%.

69. The immunomodulatory composition of paragraph 67 or 68, wherein the compound of formula (I) is dehydro-alpha-lapachone or an analog, derivative, isomer, prodrug, or pharmaceutically acceptable salt thereof.
70. Use of the anti-vascular composition of any of paragraphs 22-34 or the immunomodulatory composition of any of claims 67-69 for treatment and/or prevention of a condition involving abnormal vasculatures.
71. The use of paragraph 70, wherein the condition involving abnormal vasculatures is selected from a group consisting of tuberculosis, autoimmune diseases, wound repair, hypertrophic scar or keloid, neointimal hyperplasia, psoriasis, macular degeneration, age-related macular degeneration (AMD), thyroid hyperplasia, preeclampsia, hemangiomas, rheumatoid arthritis and osteoarthritis, Alzheimer's disease, obesity, pleural effusion, atherosclerosis, endometriosis, diabetic retinopathies, other retinopathies, ocular neovascularizations, edemas, chronic obstructive pulmonary diseases, asthma, cystic fibrosis, transplant rejection, allergic reaction, multiple sclerosis, epithelial infection, and any combinations thereof.
72. The use of paragraph 70 or 71, wherein the condition involving abnormal vasculatures includes cancer.
73. The use of paragraph 72, wherein the cancer includes a metastatic tumor.
74. The use of paragraph 73, wherein the metastatic tumor includes a metastatic breast tumor.
75. The use of paragraph 74, wherein the metastatic breast tumor includes a triple-negative breast tumor.
76. The use of paragraph 70, wherein the condition involving abnormal vasculatures excludes cancer.
77. The use of paragraph 70, wherein the condition involving abnormal vasculatures includes an ocular disease or disorder involving abnormal vasculatures.
78. The use of paragraph 77, wherein the ocular disease or disorder involving abnormal vasculatures is selected from the group consisting of age-related macular degeneration (AMD), proliferative diabetic retinopathy (PDR), diabetic macular edema (DME), neovascular glaucoma, corneal neovascularization (trachoma), and pterygium.

### Some of the Selected Definitions

As used herein, the terms “administer,” “administering,” and “administration” refer to the placement or delivery of a composition or a compound into a subject by a method or route which results in at least partial localization of the composition at a desired site such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from at least once a month, at least once a week to at least once daily depending on a number of clinical factors, such as the subject's sensitivity to the polypeptides. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks, 3 weeks, 4 weeks, 1 month, 2 months, 3 months, 4 months, 5 months, or 6 months or more.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with angiogenesis or abnormal vasculatures.

In addition, the methods described herein can be used to treat domesticated animals and/or pets. In any embodiments, a subject can be a male or a female.

In some embodiments, a subject amenable to the methods and/or compositions described herein can be one who has been previously diagnosed with or identified as suffering from or having an angiogenic- or vascular-associated disease or disorder described herein. In some embodiments, the subject can be diagnosed with or at risk of having an angiogenic-associated disease or a condition involving abnormal vasculatures described herein. In some embodiments, the subject diagnosed with or at risk of having an angiogenic- or vascular-associated disease or condition can have undergone treatment or is now receiving treatment. In some embodiments, the subject can have the angiogenic- or vascular-associated disease or disorder described herein in remission.

The term “vascular hyperproliferation” as used herein refers to abnormal vascular density resulted from aberrant proliferation or growth of blood vessels, as compared to normal vascular density in a subject without any angiogenic- or vascular-associated diseases. In some embodiments, the “vascular hyperproliferation” refer to an increase in vascular density by at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500% or higher, relative to the normal vascular density. Methods for determining vascular density are known in the art, e.g., using the methods described in the Examples.

The term “vascular hyperpermeability” as used herein refers to a process by which the fluid portion of blood leaks out of the vascular structures into the tissues of the body. In some embodiments, this leakage of fluid can cause the tissues to swell or the development of edema. In some embodiments, the “vascular hyperpermeability” refers to an increase in permeability of blood vessels by at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500% or higher, relative to the normal vascular permeability. Methods for measuring vascular permeability are well established in the art. By way of example only, in one embodiment, bovine serum albumin (BSA) and IgG can be fluorescently labeled and then either cationized by conjugation with hexamethylenediamine or anionized by succinylation. The molecules can then be injected intravenously and the fluorescence in tissue surrounding the blood vessels can be quantified by intravital fluorescence microscopy. Dellian M. 82 Br J. Cancer. 1513 (2000). In another embodiment, radioactive tracers or contrast agents can be used in place of the fluorescent-labeled molecules, and the distribution of the tracers can be then monitored using medical imaging methods.

As used herein, the term “metastatic tumor” refers to a secondary tumor that grows separately elsewhere in the body from the primary tumor and has arisen from detached, transported cells, wherein the primary tumor is a solid tumor. The primary tumor, as used herein, refers to a tumor that originated in the location or organ in which it is present and did not metastasize to that location from another location.

The term “remission” as used herein, refers to a disappearance of evidence of a disease, usually as a result of treatment. The term “complete” or “partial” can be used to modify the term “remission.” Complete remission means all evidence of the disease is gone. Partial remission means the disease is markedly improved by treatment, but residual evidence of the disease (e.g., disease-associated symptoms or disease-specific biomarker) is present. In some embodiments, partial remission is used in reference to a disease or condition not in complete remission.

The term “statistically significant” or “significantly” refers to statistical significance and generally means a two standard deviation (2 SD) below normal, or lower, concentration of the marker. The term refers to statistical evidence that there is a difference. It is defined as the probability of making a decision to reject the null hypothesis when the null hypothesis is actually true. The decision is often made using the p-value.

For simplicity, chemical moieties are defined and referred to throughout can be univalent chemical moieties (e.g., alkyl, aryl, etc.) or multivalent moieties under the appropriate structural circumstances clear to those skilled in the art. For example, an “alkyl” moiety can be referred to a monovalent radical (e.g. CH₃—CH₂—), or in other instances, a bivalent linking moiety can be “alkyl,” in which case those skilled in the art will understand the alkyl to be a divalent radical (e.g., —CH₂—CH₂—), which is equivalent to the term “alkylene.” Similarly, in circumstances in which divalent moieties are required and are stated as being “alkoxy”, “alkylamino”, “aryloxy”, “alkylthio”, “aryl”, “heteroaryl”, “heterocyclic”, “alkyl” “alkenyl”, “alkynyl”, “aliphatic”, or “cycloalkyl”, those skilled in the art will understand that the terms “alkoxy”, “alkylamino”, “aryloxy”, “alkylthio”, “aryl”, “heteroaryl”, “heterocyclic”, “alkyl”, “alkenyl”, “alkynyl”, “aliphatic”, or “cycloalkyl” refer to the corresponding divalent moiety.

The term “halogen” refers to any radical of fluorine, chlorine, bromine or iodine.

The term “acyl” refers to an alkylcarbonyl, cycloalkylcarbonyl, arylcarbonyl, heterocyclylcarbonyl, or heteroarylcarbonyl substituent, any of which may be further substituted by substituents. Exemplary acyl groups include, but are not limited to, (C1-C6)alkanoyl (e.g., formyl, acetyl, propionyl, butyryl, valeryl, caproyl, t-butylacetyl, etc.), (C3-C6)cycloalkylcarbonyl (e.g., cyclopropylcarbonyl, cyclobutylcarbonyl, cyclopentylcarbonyl, cyclohexylcarbonyl, etc.), heterocyclic carbonyl (e.g., pyrrolidinylcarbonyl, pyrrolid-2-one-5-carbonyl, piperidinylcarbonyl, piperazinylcarbonyl, tetrahydrofuranylcarbonyl, etc.), aroyl (e.g., benzoyl) and heteroaroyl (e.g., thiophenyl-2-carbonyl, thiophenyl-3-carbonyl, furanyl-2-carbonyl, furanyl-3-carbonyl, 1H-pyrroyl-2-carbonyl, 1H-pyrroyl-3-carbonyl, benzo[b]thiophenyl-2-carbonyl, etc.). In addition, the alkyl, cycloalkyl, heterocycle, aryl and heteroaryl portion of the acyl group may be any one of the groups described in the respective definitions.

The term “alkyl” refers to saturated non-aromatic hydrocarbon chains that may be a straight chain or branched chain, containing the indicated number of carbon atoms (these include without limitation propyl, allyl, or propargyl), which may be optionally inserted with N, O, or S. For example, C1-C6 indicates that the group may have from 1 to 6 (inclusive) carbon atoms in it.

The term “alkenyl” refers to an alkyl that comprises at least one double bond. Exemplary alkenyl groups include, but are not limited to, for example, ethenyl, propenyl, butenyl, 1-methyl-2-buten-1-yl and the like.

The term “alkynyl” refers to an alkyl that comprises at least one triple bond.

The term “aryl” refers to monocyclic, bicyclic, or tricyclic aromatic ring system wherein 0, 1, 2, 3, or 4 atoms of each ring may be substituted by a substituent. Exemplary aryl groups include, but are not limited to, phenyl, naphthyl, anthracenyl, azulenyl, fluorenyl, indanyl, indenyl, naphthyl, phenyl, tetrahydronaphthyl, and the like.

The term “cyclyl” or “cycloalkyl” refers to saturated and partially unsaturated cyclic hydrocarbon groups having 3 to 12 carbons, for example, 3 to 8 carbons, and, for example, 3 to 6 carbons, wherein the cycloalkyl group additionally may be optionally substituted. Exemplary cycloalkyl groups include, but are not limited to, cyclopropyl, cyclobutyl, cyclopentyl, cyclopentenyl, cyclohexyl, cyclohexenyl, cycloheptyl, cyclooctyl, and the like.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, and the like.

The term “heterocyclyl” refers to a nonaromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. Exemplary heterocyclyl groups include, but are not limited to piperazinyl, pyrrolidinyl, dioxanyl, morpholinyl, tetrahydrofuranyl, and the like.

The term “haloalkyl” refers to an alkyl group having one, two, three or more halogen atoms attached thereto. Exemplary haloalkyl groups include, but are not limited to chloromethyl, bromoethyl, trifluoromethyl, and the like.

The term “optionally substituted” means that the specified group or moiety, such as an alkyl group, alkenyl group, alkynyl group, cyclyl group, heterocyclyl group, aryl group, heteroaryl group and the like, is unsubstituted or is substituted with one or more (typically 1-4 substituents) independently selected from the group of substituents listed below in the definition for “substituents” or otherwise specified.

The term “substituents” refers to a group “substituted” on an alkyl, alkenyl, alkynyl, cycloalkyl, aryl, heterocyclyl, or heteroaryl group at any atom of that group. Suitable substituents include, without limitation, halo, hydroxy, oxo, nitro, haloalkyl, alkyl, alkenyl, alkynyl, alkaryl, aryl, aralkyl, alkoxy, aryloxy, amino, acylamino, alkylcarbanoyl, arylcarbanoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano or ureido. In some cases, two substituents, together with the carbons to which they are attached to can form a ring.

In many cases, protecting groups are used during preparation of the compounds described herein. As used herein, the term “protected” means that the indicated moiety has a protecting group appended thereon. In some embodiments, compounds contain one or more protecting groups. A wide variety of protecting groups can be employed in the methods described herein. In general, protecting groups render chemical functionalities inert to specific reaction conditions, and can be appended to and removed from such functionalities in a molecule without substantially damaging the remainder of the molecule.

Representative protecting groups, are disclosed in Greene and Wuts, Protective Groups in Organic Synthesis, Chapter 2, 2d ed., John Wiley & Sons, New York, 199. Examples of hydroxyl protecting groups include, but are not limited to, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p,p′-dinitrobenzhydryl, p-nitrobenzyl, triphenylmethyl, trimethylsilyl, triethylsilyl, t-butyldimethylsilyl, t-butyldiphenylsilyl, triphenylsilyl, benzoylformate, acetate, chloroacetate, trichloroacetate, trifluoroacetate, pivaloate, benzoate, p-phenylbenzoate, 9-fluorenylmethyl carbonate, mesylate and tosylate. Exemplary amino-protecting groups include, but are not limited to, carbamate protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyloxycarbonyl (Cbz); amide protecting groups, such as formyl, acetyl, trihaloacetyl, benzoyl, and nitrophenylacetyl; sulfonamide protecting groups, such as 2-nitrobenzenesulfonyl; and imine and cyclic imide protecting groups, such as phthalimido and dithiasuccinoyl.

As used here in the term “isomer” refers to compounds having the same molecular formula but differing in structure. Isomers which differ only in configuration and/or conformation are referred to as “stereoisomers.” The term “isomer” is also used to refer to an enantiomer.

The term “enantiomer” is used to describe one of a pair of molecular isomers which are mirror images of each other and non-superimposable. Other terms used to designate or refer to enantiomers include “stereoisomers” (because of the different arrangement or stereochemistry around the chiral center; although all enantiomers are stereoisomers, not all stereoisomers are enantiomers) or “optical isomers” (because of the optical activity of pure enantiomers, which is the ability of different pure enantiomers to rotate plane-polarized light in different directions). Enantiomers generally have identical physical properties, such as melting points and boiling points, and also have identical spectroscopic properties. Enantiomers can differ from each other with respect to their interaction with plane-polarized light and with respect to biological activity.

The designations “R and S” are used to denote the absolute configuration of the molecule about its chiral center(s). The designations may appear as a prefix or as a suffix; they may or may not be separated from the isomer by a hyphen; they may or may not be hyphenated; and they may or may not be surrounded by parentheses.

The designations or prefixes “(+) and (−)” are employed to designate the sign of rotation of plane-polarized light by the compound, with (−) meaning that the compound is levorotatory (rotates to the left). A compound prefixed with (+) is dextrorotatory (rotates to the right).

The term “racemic mixture,” “racemic compound” or “racemate” refers to a mixture of the two enantiomers of one compound. An ideal racemic mixture is one wherein there is a 50:50 mixture of both enantiomers of a compound such that the optical rotation of the (+) enantiomer cancels out the optical rotation of the (−) enantiomer.

The term “analog” as used herein refers to a compound that results from substitution, replacement or deletion of various organic groups or hydrogen atoms from a parent compound. As such, some monoterpenoids can be considered to be analogs of monoterpenes, or in some cases, analogs of other monoterpenoids, including derivatives of monoterpenes. An analog is structurally similar to the parent compound, but can differ by even a single element of the same valence and group of the periodic table as the element it replaces.

The term “derivative” as used herein refers to a chemical substance related structurally to another, i.e., an “original” substance, which can be referred to as a “parent” compound. A “derivative” can be made from the structurally-related parent compound in one or more steps. The phrase “closely related derivative” means a derivative whose molecular weight does not exceed the weight of the parent compound by more than 50%. The general physical and chemical properties of a closely related derivative are also similar to the parent compound.

As used herein, a “prodrug” refers to compounds that can be converted via some chemical or physiological process (e.g., enzymatic processes and metabolic hydrolysis) to a therapeutic agent. Thus, the term “prodrug” also refers to a precursor of a biologically active compound that is pharmaceutically acceptable. A prodrug may be inactive when administered to a subject, i.e. an ester, but is converted in vivo to an active compound, for example, by hydrolysis to the free carboxylic acid or free hydroxyl. The prodrug compound often offers advantages of solubility, tissue compatibility or delayed release in an organism. The term “prodrug” is also meant to include any covalently bonded carriers, which release the active compound in vivo when such prodrug is administered to a subject. Prodrugs of an active compound may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a subject, cleaves to form a free hydroxy, free amino or free mercapto group, respectively. Examples of prodrugs include, but are not limited to, acetate, formate and benzoate derivatives of an alcohol or acetamide, formamide and benzamide derivatives of an amine functional group in the active compound and the like. See Harper, “Drug Latentiation” in Jucker, ed. Progress in Drug Research 4:221-294 (1962); Morozowich et al, “Application of Physical Organic Principles to Prodrug Design” in E. B. Roche ed. Design of Biopharmaceutical Properties through Prodrugs and Analogs, APHA Acad. Pharm. Sci. 40 (1977); Bioreversible Carriers in Drug in Drug Design, Theory and Application, E. B. Roche, ed., APHA Acad. Pharm. Sci. (1987); Design of Prodrugs, H. Bundgaard, Elsevier (1985); Wang et al. “Prodrug approaches to the improved delivery of peptide drug” in Curr. Pharm. Design. 5(4):265-287 (1999); Pauletti et al. (1997) Improvement in peptide bioavailability: Peptidomimetics and Prodrug Strategies, Adv. Drug. Delivery Rev. 27:235-256; Mizen et al. (1998) “The Use of Esters as Prodrugs for Oral Delivery of (3-Lactam antibiotics,” Pharm. Biotech. 11:345-365; Gaignault et al. (1996) “Designing Prodrugs and Bioprecursors I. Carrier Prodrugs,” Pract. Med. Chem. 671-696; Asgharnejad, “Improving Oral Drug Transport”, in Transport Processes in Pharmaceutical Systems, G. L. Amidon, P. I. Lee and E. M. Topp, Eds., Marcell Dekker, p. 185-218 (2000); Balant et al., “Prodrugs for the improvement of drug absorption via different routes of administration”, Eur. J. Drug Metab. Pharmacokinet., 15(2): 143-53 (1990); Balimane and Sinko, “Involvement of multiple transporters in the oral absorption of nucleoside analogues”, Adv. Drug Delivery Rev., 39(1-3): 183-209 (1999); Browne, “Fosphenyloin (Cerebyx)”, Clin. Neuropharmacol. 20(1): 1-12 (1997); Bundgaard, “Bioreversible derivatization of drugs—principle and applicability to improve the therapeutic effects of drugs”, Arch. Pharm. Chemi 86(1): 1-39 (1979); Bundgaard H. “Improved drug delivery by the prodrug approach”, Controlled Drug Delivery 17: 179-96 (1987); Bundgaard H. “Prodrugs as a means to improve the delivery of peptide drugs”, Arfv. Drug Delivery Rev. 8(1): 1-38 (1992); Fleisher et al. “Improved oral drug delivery: solubility limitations overcome by the use of prodrugs”, Arfv. Drug Delivery Rev. 19(2): 115-130 (1996); Fleisher et al. “Design of prodrugs for improved gastrointestinal absorption by intestinal enzyme targeting”, Methods Enzymol. 112 (Drug Enzyme Targeting, Pt. A): 360-81, (1985); Farquhar D, et al., “Biologically Reversible Phosphate-Protective Groups”, Pharm. Sci., 72(3): 324-325 (1983); Freeman S, et al., “Bioreversible Protection for the Phospho Group: Chemical Stability and Bioactivation of Di(4-acetoxy-benzyl) Methylphosphonate with Carboxyesterase,” Chem. Soc., Chem. Commun., 875-877 (1991); Friis and Bundgaard, “Prodrugs of phosphates and phosphonates: Novel lipophilic alphaacyloxyalkyl ester derivatives of phosphate- or phosphonate containing drugs masking the negative charges of these groups”, Eur. J. Pharm. Sci. 4: 49-59 (1996); Gangwar et al., “Pro-drug, molecular structure and percutaneous delivery”, Des. Biopharm. Prop. Prodrugs Analogs, [Symp.] Meeting Date 1976, 409-21. (1977); Nathwani and Wood, “Penicillins: a current review of their clinical pharmacology and therapeutic use”, Drugs 45(6): 866-94 (1993); Sinhababu and Thakker, “Prodrugs of anticancer agents”, Adv. Drug Delivery Rev. 19(2): 241-273 (1996); Stella et al., “Prodrugs. Do they have advantages in clinical practice?”, Drugs 29(5): 455-73 (1985); Tan et al. “Development and optimization of anti-HIV nucleoside analogs and prodrugs: A review of their cellular pharmacology, structure-activity relationships and pharmacokinetics”, Adv. Drug Delivery Rev. 39(1-3): 117-151 (1999); Taylor, “Improved passive oral drug delivery via prodrugs”, Adv. Drug Delivery Rev., 19(2): 131-148 (1996); Valentino and Borchardt, “Prodrug strategies to enhance the intestinal absorption of peptides”, Drug Discovery Today 2(4): 148-155 (1997); Wiebe and Knaus, “Concepts for the design of anti-HIV nucleoside prodrugs for treating cephalic HIV infection”, Adv. Drug Delivery Rev.: 39(1-3):63-80 (1999); Waller et al., “Prodrugs”, Br. J. Clin. Pharmac. 28: 497-507 (1989), content of all of which is herein incorporated by reference in its entirety.

As used herein, the term “pharmaceutically-acceptable salts” refers to the conventional nontoxic salts or quaternary ammonium salts of therapeutic agents, e.g., from non-toxic organic or inorganic acids. These salts can be prepared in situ in the administration vehicle or the dosage form manufacturing process, or by separately reacting a therapeutic agent in its free base or acid form with a suitable organic or inorganic acid or base, and isolating the salt thus formed during subsequent purification. Conventional nontoxic salts include those derived from inorganic acids such as sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. See, for example, Berge et al., “Pharmaceutical Salts”, J. Pharm. Sci. 66:1-19 (1977), content of which is herein incorporated by reference in its entirety.

In some embodiments of the aspects described herein, representative salts include the hydrobromide, hydrochloride, sulfate, bisulfate, phosphate, nitrate, acetate, succinate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

In one respect, the present invention relates to the herein described compositions, methods, and respective component(s) thereof, as essential to the invention, yet open to the inclusion of unspecified elements, essential or not (“comprising). In some embodiments, other elements to be included in the description of the composition, method or respective component thereof are limited to those that do not materially affect the basic and novel characteristic(s) of the invention (“consisting essentially of”). This applies equally to steps within a described method as well as compositions and components therein. In other embodiments, the inventions, compositions, methods, and respective components thereof, described herein are intended to be exclusive of any element not deemed an essential element to the component, composition or method (“consisting of”).

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages may mean±1%. The present invention is further explained in detail by the following examples, but the scope of the invention should not limit thereto.

### EXAMPLES

The examples presented herein relate to methods for screening an anti-vascular agent from a compound library and demonstration of anti-vascular activity of the identified compound described herein, e.g., dehydro-alpha-lapachone. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

### Materials and Methods

Compound Library.

A compound library from the Partners Center for Drug Discovery (PCDD) was used for a high-throughput assay. This library includes 50,000 compounds from different sources: i) marketed drugs from Prestwick; ii) purified natural products; iii) 3000 end-blocked tetrapeptides; iv) small molecules purchased from Peakdale, Maybridge, CEREP, Bionet Research Ltd., Chemical Diversity Lab, ENAMINE, and I.F. Lab Ltd; and v) small molecules from PCDD chemists and from different academic institutions. Compounds were stored at a stock concentration of 2 mg/ml to approximately 5 mM.

For the screen, the 5 mM solutions were diluted to approximately 1.67 mM in 100% DMSO, and 0.4 μL of each was spotted in wells in 384-well plates using a Beckman Coulter Multimek 96/384 Channel Automated Pipetter. The average molecular weight of the compounds in the library is 400 Da (range=225-600), and this library has been tested successfully in a number of screening assays.

High-Throughput Screening (HTS).

A cell-based readout system was used for compound selection. For the cell-based screening, an optimal cell density of 10,000 breast cancer MDA-MB-231 cells/well was selected to produce the most prominent signal. The plated cells were incubated with each library compound, washed with PBS, and the Cell Titer-Glo (Promega) reagent was used to detect the number of cells remaining attached in each well. This ATP-based detection has a linear relationship between the luminescent signal and the number of cells per well. Of the 50,000 compounds screened, 86 library compounds (0.172% of screened compounds) were selected at 1.67 μM concentration for the second screening step. Breast cancer cells were treated with each of the selected library compounds at 3 μM for 3 hours and then examined for changes in cell shape by microscopy. Twelve of the 86 compounds were selected for further study. All these selected compounds were then passed through a number of toxicity assays to eliminate any toxic compounds. The absence of toxicity for the candidate compounds was confirmed in the cellular model in repetitive experiments and re-assayed at three different concentrations (1.67 μM, 5 μM and 10 μM). The compounds that showed no toxicity at any concentrations tested were analyzed in a zebrafish model, and the compounds at concentrations that did not affect zebrafish survival during embryonic development and adult life were used for further experiments. Of the 50,000 compounds, the compound dehydro-α-lapachone (DAL) was selected for further characterization. As a further test of toxicity, DAL was injected intraperitoneally at 100 mg/kg in SCID mice and signs of toxicity including cachexia, ruffled fur, diarrhea, anorexia, skin ulceration, and toxic death were monitored.

Zebrafish Maintenance and Drug Exposure.

Zebrafish were raised in accordance with the established protocols (19-20). The age of embryos is indicated by the hours after fertilization and days after fertilization for all experimental data shown herein. For the screening study, zebrafish embryos were incubated with the selected compounds at a concentration of 5-10 μM. Treated embryos were then examined under a 100× PlanNeofluor objective mounted on a Nikon TE-200 epifluorescence microscope. Images of embryos bearing GFP-positive cells were captured with a Zeiss stereomicroscope.

Regeneration Experiments.

For the regeneration experiments, caudal fins of zebrafish were amputated at approximately the 50% proximal-distal level (21). The amputated fish were kept in individual 250-ml fish tanks, and at least five fish were used in each group. Staging for normal blood vessel regeneration was carried out at 25° C. with TG(fli1:EGFP)y1 fish, with the same fin ray and vessels of each fish was photographed at different time intervals during the course of the time-lapse study.

Determination of Rac1 Activity.

Rac1 and cdc42 activities were measured with a Rac1 (BK035) activation assay biochem kit (Cytoskeleton) according to the manufacturer protocol. In brief, human umbilical vein endothelial cells (HUVECs) were bovine brain extract-starved for 12 hours in 1% FBS-EGM (Lonza) and pretreated with various concentrations of DAL for 30 minutes at 37° C. The HUVECs were then stimulated with VEGF at 50 ng/μl for 15 minutes at 37° C. After stimulation, the cells were immediately incubated on ice, washed twice with PBS, and lysed with lysis buffer. The lysates were pre-cleared and added with 15 μl of PAK-PBD beads for pull-down of activated Rac1 or cdc42. After rocking at 4° C. for 1 hour, the beads were washed once and boiled at 100° C. for 2 minutes. The protein lysates were then resolved in a 8-16% gradient gel (Invitrogen) and transferred to a PVDF membrane. Primary antibodies (anti-Rac1, 1:1000, cytoskeleton or anti-cdc42, 1:1000, cell signaling) were added to bind to the protein overnight at 4° C., followed by incubation with HRP-conjugated secondary antibodies. Protein activity was measured by the standard ECL method (Amersham).

Measurement of Vascular Parameters in Tumors in Mice.

Female SCID mice bearing orthotopic GFP-labeled 3 mm 4T1 mammary tumors were treated with five daily i.p. injections (day 0-day 4) of 37.5 mg/kg DAL or saline. Implanted mammary fat pad windows (35) were surgically implanted to facilitate intravital imaging through multiphoton microscopy with a custom-built multiphoton microscope with a 20×0.95NA objective (Olympus), and mice were imaged by fluorescent angiography following retro-orbital injection of 60 uL of 10 mg/mL TAMRA-dextran (2MDa molecular mass). Images were collected on day 0, day 2, and day 4, with the first images collected immediately before the first treatment. The 2×2 grids were imaged in the sample plane (630-μm) field of view at each position) tissue from 0-200 μm depth. To measure vascular parameters in the mice over the course of treatment, a custom 3D vascular tracing and analysis software package based on MATLAB (The Mathworks) was applied to the images.

Tumor Growth Delay Study.

To assess the anti-tumor effects of DAL, female SCID mice bearing orthotopic 4T1 or E0771 mammary tumors were treated with daily i.p. injections of 37.5 mg/kg DAL or saline. In order to calculate tumor volumes, the major and minor axis diameters were measured with calipers and the tumor volume was calculated by assuming a spherical shape based on the average of these two dimensions. The tumors were implanted in the mammary fat pad and allowed to grow to a starting size (small ˜100 mm³ tumors for E0771, large ˜200 mm³ tumors for 4T1) over ˜2 weeks. To ensure that tumors in the two groups were of similar growth rates, the treatment with saline or DAL was begun in pairs of tumors with similar sizes and growth times. Tumor volumes were measured every other day until the tumors reached an average of ˜10 mm in diameter. Tumor growth was quantified with the doubling rate, and calculated by fitting an exponential growth curve to the data for tumor size versus time.

Kinase Inhibition Screening.

To determine kinase activity of DAL, various kinases (AKT1, AKT2, AKT3, PKA, PKC alpha, PKC delta, PAK1, EPHB2, EPHB4, p38 alpha, PI3K-alpha, PI3K-beta, PI3K-gamma, PI3K-delta) were screened in a 10-dose IC₅₀ mode with threefold serial dilutions starting at 100 μM. Staurosporine was used as a control starting at 10 μM and the reactions were carried out at 1 μM ATP.

### Example 1

### Identification of DAL from a Compound Library

A screening strategy was developed to identify potential agents that target adhesion of endothelial cells or cancer cells to their substrate. To this end, 50,000 compounds were first screened in a high-throughput manner (FIG. 1A). The initial screening step quantified the number of cells remaining attached to their wells following incubation with each compound and subsequent washing steps. Only 86 compounds affected cellular adhesion in the assay. Cell adhesion adaptor proteins have a domain for binding actin filaments (15-16), thus adhesion molecules are directly linked to the actin cytoskeleton. The agents that affect cell adhesion can be monitored through remodeling of actin filaments. As the second screening step, the effect of the selected compounds on actin assembly and redistribution was assessed by fixing and staining treated cells with phalloidin. Changes in actin assembly and cell shape after treatment with 12 compounds were determined. This set of compounds was next prioritized for their nontoxicity to normal cells, cancer cells and zebrafish. Of these 12 compounds, two were selected based on the in vitro and in vivo assessment. One of these compounds is dehydro-alpha-lapachone (DAL; FIG. 1B). It is previously showed that beta-lapachone, with a distinct chemical structure from DAL, has antitumor and anti-trypanosomal activities through DNA topoisomerase I inhibition and prevention of DNA repair (17-18). DAL was further assessed for toxicity in SCID mice, and it was determined that mice are tolerant to intraperitoneal doses of up to 100 mg/kg with no signs of toxicity. Therefore, DAL—a natural product from the Tabebuia Avellaneda tree—was selected for further study.

### Example 2

### Antivascular Effects of DAL in Zebrafish Models

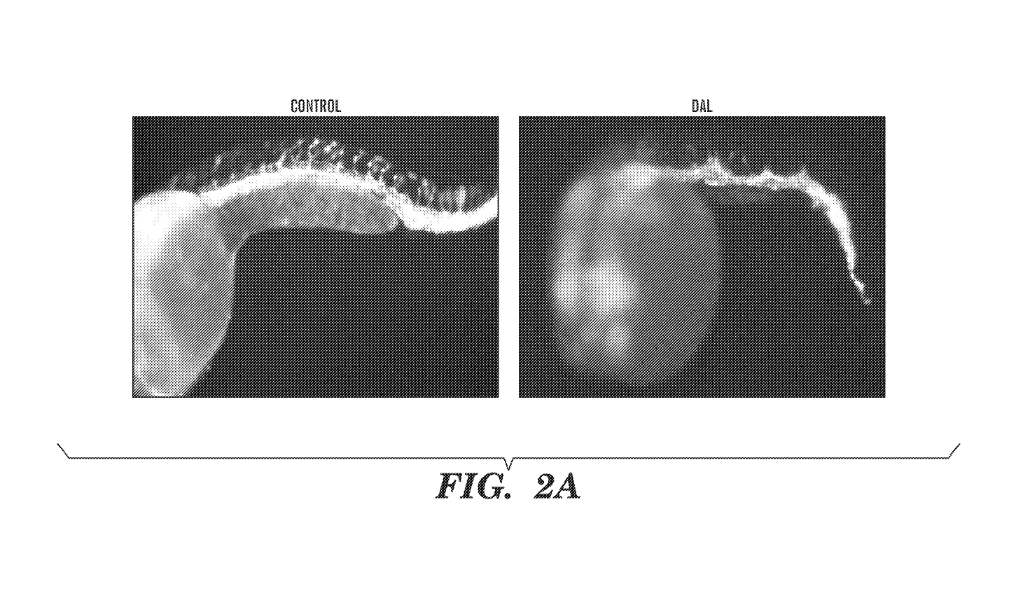

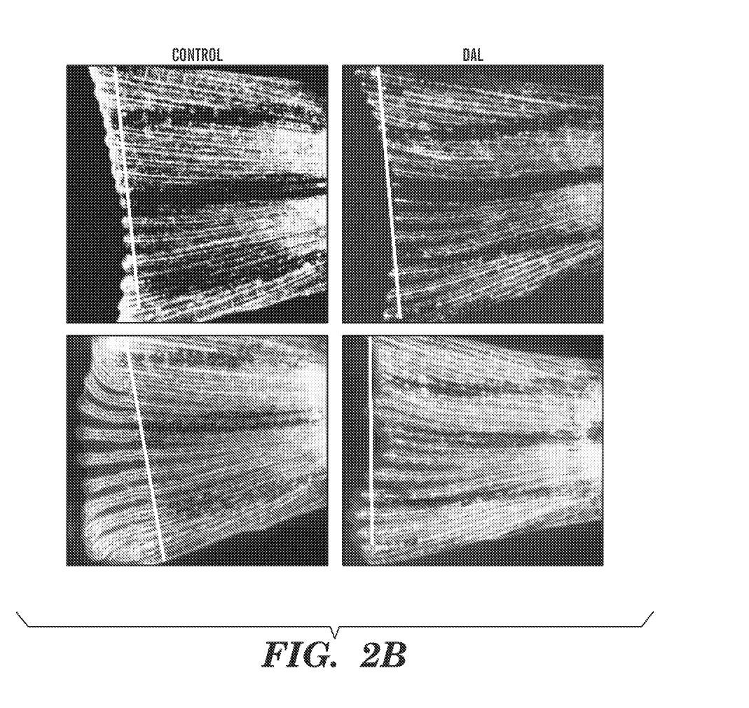

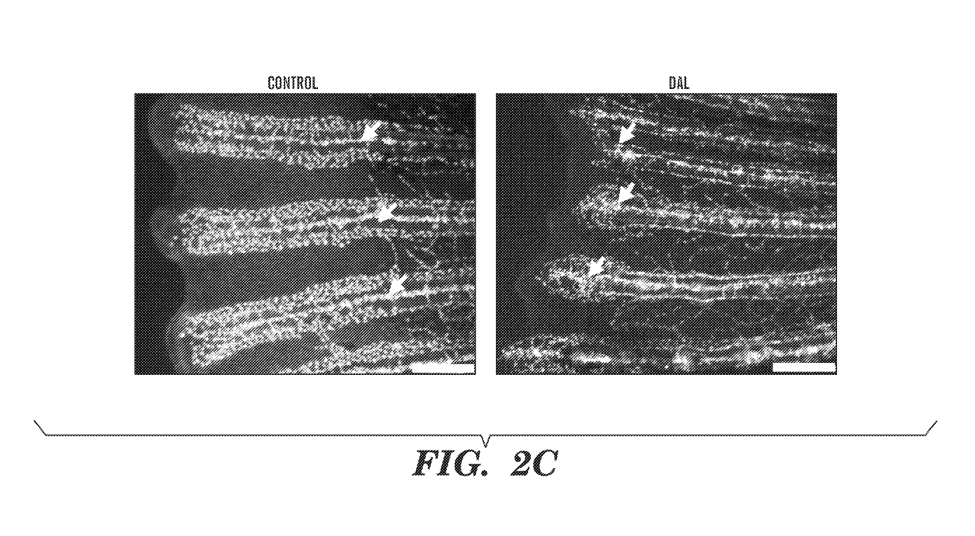

To elucidate the potential impact of DAL on the process of vascular network formation, its effects in zebrafish embryos at different stages of development were assessed. To visualize vessel defects, transgenic fish expressing EGFP in endothelial cells (Tg(fli1:EGFP)^{y1}) were used (19-20). This model expresses EGFP in blood vessel ECs throughout normal development and during fin regeneration (21). The effects of DAL on normal vascular development in zebrafish embryos were first characterized. In control embryos, developing vessels migrated from the lateral plate mesoderm to the midline, where they coalesced into a vascular cord. These endothelial clusters subsequently established the pattern of the dorsal aorta and posterior cardinal vein. Intersomitic vessels sprouted at designated branch sites in control embryos after dorsal aorta formation. In contrast, after treatment with DAL, the sprouting of intersomitic vessels to their designated branch sites failed to occur and the embryos developed unusual wave-like vessel structures (FIG. 2A). DAL treatment resulted in a profound reduction in the complexity of the arterial network. These findings demonstrate that DAL can interfere with the regulation of vascular formation and branching morphogenesis during development. DAL did not show any effects on the vasculature in adult fish, and it is believed that it can be due to the stable junctions and postmitotic nature of the mature vasculature.

To examine whether DAL has a similar antivascular effect on neovasculature in adult zebrafish, neovascularization was induced by amputating the caudal fin at the ˜50% proximal-distal level and fin rays and vessels were imaged from each fish over time. In control zebrafish caudal fins, amputated blood vessels healed their ends by 24 h post-amputation and then reconnected arteries and veins through anastomosis, with blood flow resuming at wound sites by 48 h after amputation (FIG. 2B). Meanwhile, regenerating vessels in fish treated with DAL had defects in anastomosis and plexus formation. All control fish developed normal blood vessels and formed anastomotic bridges at the amputation plane by 9 days after amputation; in contrast, we did not find such bridges in fish treated with DAL (FIG. 2C).

### Example 3

### DAL Prunes Tumor Vasculature

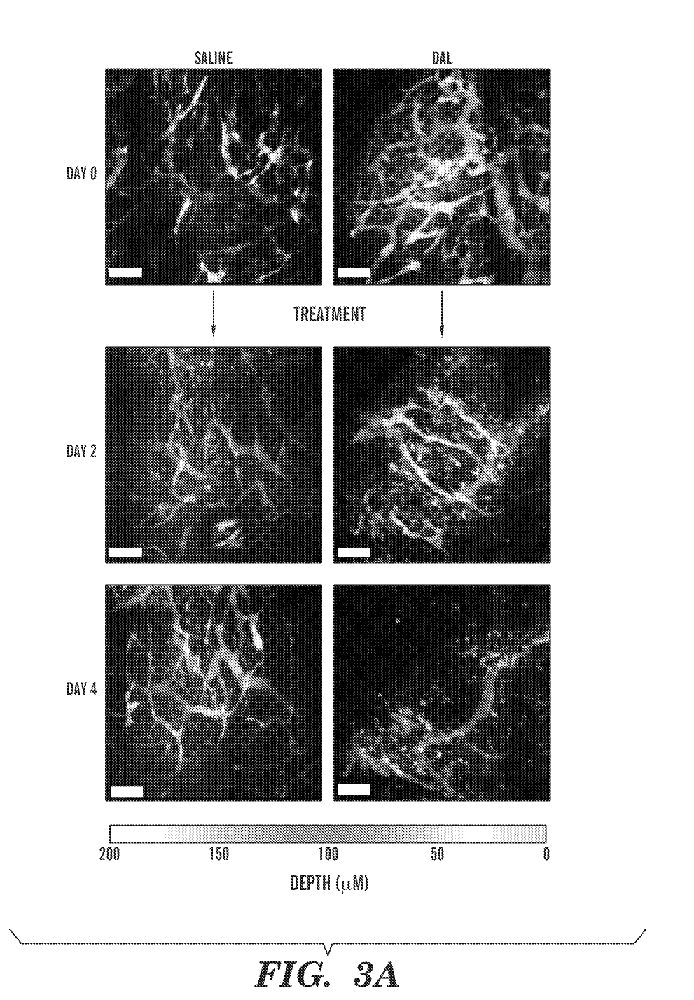

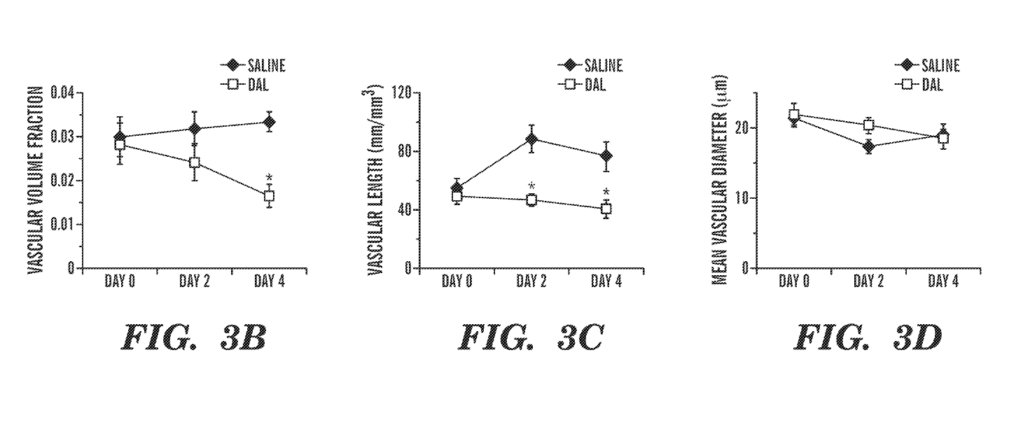

Since the zebrafish data indicated that DAL is a potential antivascular agent selective for vasculature, it was sought to determine the effects of DAL on tumor vasculature in mammals. To study these effects quantitatively, fluorescent angiographies were conducted in female SCID mice bearing orthotopic 4T1 mammary tumors in mammary fat pad windows via intravital multiphoton microscopy (FIG. 3A) (22-24). These mice were treated with 37.5 mg/kg DAL or saline daily for 5 days by i.p. injection. It was determined that DAL treatment decreases tumor vascular volume fractions—a measure of vascular density—compared with saline treatment (FIG. 3B, p<0.002, day 4). Furthermore, DAL treatment lowers total tumor vascular length (normalized to tumor volume) versus saline treatment (FIG. 3C, p=0.007, day 2; p=0.02, day 4), whereas mean tumor vascular diameter remains the same (FIG. 3D). These data indicate that DAL reduces vascular density in tumors through vessel pruning.

### Example 4

### DAL Inhibits Tumor Growth

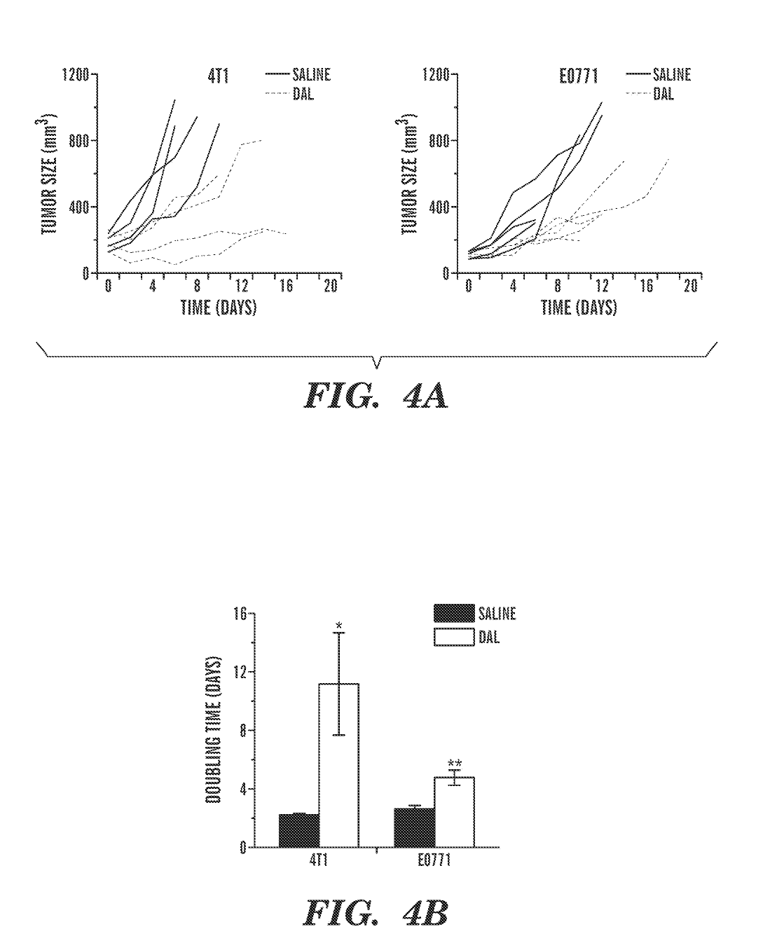

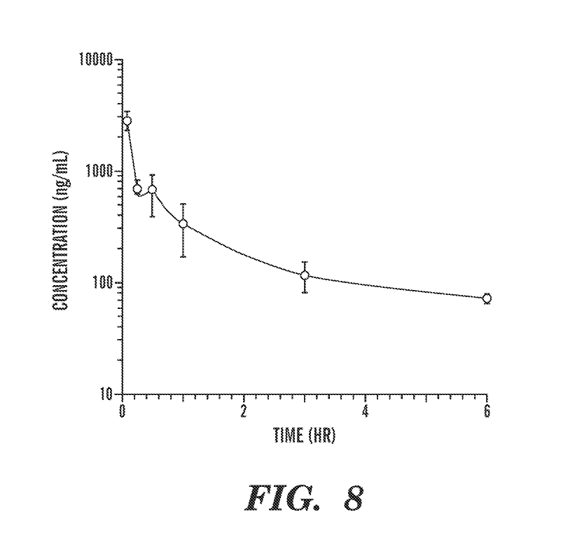

As shown in Example 3, DAL has an antivascular effect in orthotopic mammary tumors in mice. Thus, the antitumor effect of DAL was next assessed. Pharmacokinetic studies for DAL indicated that its half-life is 1.7 hours in plasma after i.p. dosing (FIG. 8). The effect of DAL on the growth of orthotopic 4T1 and E0771 mammary tumors with daily treatment of DAL or saline was evaluated (FIG. 4A). 37.5 mg/kg of DAL was used for these experiments based on the pharmacokinetics results as well as in vitro and in vivo data. In E0771 tumors, with treatment initiated at a ˜100 mm³ volume, DAL increased the tumor volume doubling time from 2.65+/−0.27 days to 4.77+/−0.58 days (p=0.01). In 4T1 tumors, with treatment starting at a larger tumor volume of ˜200 mm³, DAL increased the doubling time from 2.20+/−0.07 days to 11.21+/−3.53 days (p=0.04, FIG. 4B). Further, DAL induced no weight loss or other noted signs of toxicity in these mice. Together, these results indicate that DAL is a safe chemical agent with prominent anti-tumor effect.

### Example 5

### DAL Interferes with Adhesive Properties of Endothelial Cells

To gain further insight into the anti-vascular effects of DAL and to assess the involvement of DAL in modulating actin assembly, normal human umbilical vein endothelial cells(HUVEC) were plated and treated with 10 μM DAL for three hours. The cells were fixed, permeabilized, and subsequently stained with phalloidin to visualize F-actin (FIG. 5A). Cells treated with DAL were smaller than untreated control cells and had a rounded shape. Actin stress fibers were clearly visible and aligned with the long axis in control cells, while DAL treated cells featured actin present only as small fibers and spots throughout the cells or accumulated close to the nuclei. Together, these results show that DAL destroys or significantly changes the normal organization of the actin cytoskeleton and further inhibits actin-dependent processes such as cell spreading on the extracellular matrix.

To assess whether DAL affects EC functions, in vitro models of vascular network formation from HUVECs were used. After overnight cultivation in matrigel, HUVECs spontaneously assemble into cord-like structures that resemble vascular networks. Treatment with DAL not only blocks HUVEC network formation, but also leads to complete reorganization of existing networks over a few hours (FIG. 5B). These results indicate that DAL can interfere with cell-cell junction integrity and actin assembly in ECs as a mechanism for its anti-vascular effects. Since DAL affects actin cytoskeleton assembly, the effect of DAL on cell motility was next assessed. A scratch wound assay was utilized to study HUVEC motility based on the healing rate of a wounded monolayer. Cells treated with DAL did not move and failed to close the wound (FIG. 5C). These results indicate that DAL can also interfere with EC motility, indicating a secondary mechanism for inhibiting angiogenic sprouting.

### Example 6

### DAL Decreases Rac1 Activity and Promotes Degradation

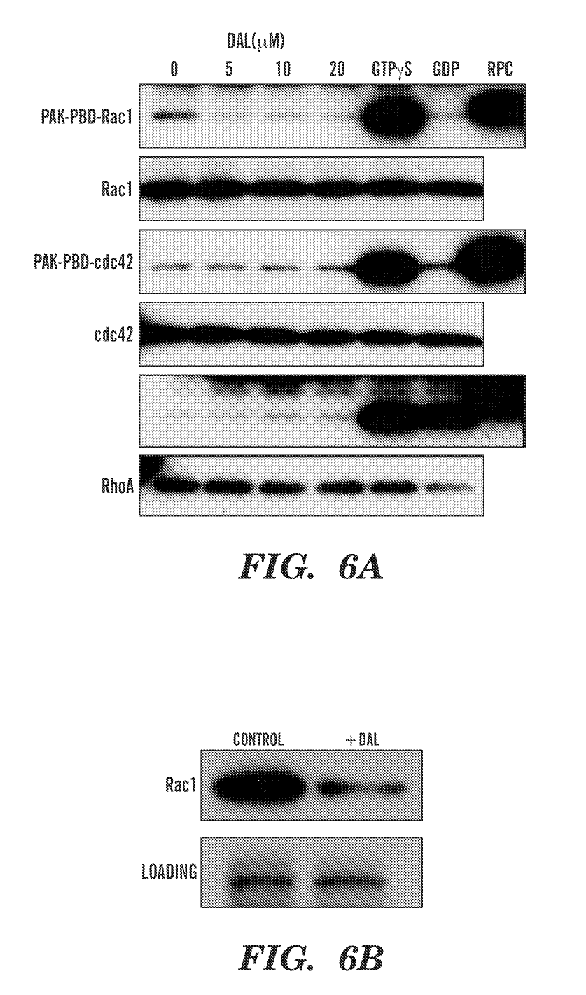

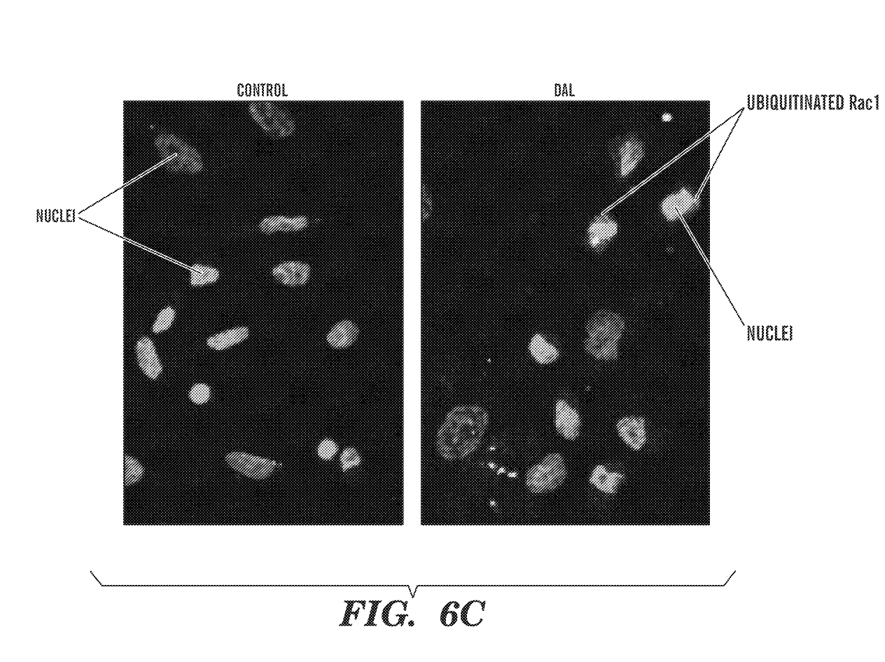

Without wishing to be bound by theory, DAL brings about its antivascular effects by destabilizing cell adhesion through the actin cytoskeleton. It was next sought to identify which specific adhesion pathway it targets. Mechanical attachment at cell-cell adherens and tight junctions is regulated by dynamic changes in the actin cytoskeleton. Adherens junctions are composed of E-cadherin/β-catenin complexes and are connected to the actin cytoskeleton via sa-catenin (25-26), while tight junctions consist of transmembrane claudins and occludin and are associated with the actin cytoskeleton via ZO proteins (27-28). Therefore, potential targets of DAL in signaling pathways involved in actin remodeling were evaluated. It was determined that DAL had no significant effects on 14 kinases (AKT1, AKT2, AKT3, PKA, PKC alpha, PKC delta, PAK1, EPHB2, EPHB4, p38 alpha, PI3K-alpha, PI3K-beta, PI3K-gamma, PI3K-delta). Then, it was sought to determine whether Rho-GTPase family members are targets for DAL due to the disruption of cell adhesion and actin network remodeling along with inhibited motility. Rho-GTPases (e.g., Rac1) plays an important role in actin cytoskeletal organization and is involved in the coordination of cell adhesion (29). Furthermore, Rac1 is responsible for stabilizing endothelial cell junctions by opposing actin remodeling by Rho (30). In order to migrate, cancer cells employ dynamic remodeling of the actin cytoskeleton, form protrusive structures and generate forces necessary to produce cell translocation in the process regulated by Rho-GTPase Rac1. To assess whether DAL can modulate Rac1, RhoA, and Cdc42 activities, affinity purification assays were employed to monitor RhoA and Cdc42/Rac1 activity. The Rho-Binding Domain (RBD) of the Rho effector protein rhotekin and the p21 Binding Domain (PBD) of the Cdc42/Rac effector protein p21 activated kinase 1 (PAK) were applied to pull down active forms of each protein. It was determined that DAL decreased Rac1 activity in a concentration-dependent manner (FIG. 6A)—and can decrease Rac1 levels—but had no effect on Cdc42 or RhoA activity, indicating Rac1 regulators or Rac1 itself as potential targets of DAL.

Treatment of cells with DAL did not change expression levels of Rac1 mRNA measured with quantitative PCR. However, protein levels of Rac1 were reduced after DAL treatment (FIG. 6B). Next, it was assessed whether DAL can enhance Rac1 ubiquitination and therefore promote or speed up degradation (31-34). For the detection of Rac1 ubiquitination in HUVECs, Rac1-ubiquitin binding in situ was identified using a Duolink molecular proximity assay. In this assay, molecules thought to interact are targeted with fluorescent antibodies in situ, and individual interactions generate a signal detectable by microscopy only if secondary antibodies are located in close proximity to each other. For detection of ubiquitinated Rac1 proteins, a mouse antibody raised against Rac1 protein and a rabbit antibody raised against an ubiquitin were used. Low concentrations of irreversible (lactacystin) and reversible (MG-132) proteasome inhibitors were used to block the ubiquitin proteasome system and enrich the fraction of ubiquitinated proteins in the assay. Cells treated with DAL contained more ubiquitinated Rac1 protein compared to control cells (FIG. 6C). These results indicate that, without wishing to be bound by theory, DAL promotes Rac1 ubiquitination, leading to rapid Rac1 degradation.

### Example 7

### Effect of DAL on Immunomodulation

Tumor cells are under strong selective pressure to develop the capacity to undermine an effective immune response. A growing body of evidence indicates that cancer patients have increased tryptophan catabolism through elevation of the enzyme indoleamine-2,3-dioxygenase (IDO), which has immunosuppressive characteristics (Katz et al., 2008; Mellor and Munn, 2004; Munn and Mellor, 2007). Therefore, IDO1 can be an attractive therapeutic target for pharmacological intervention in cancer as well as other diseases in which effective immunity is impaired. IDO catalyzes tryptophan degradation in the initial step of the kynurenine pathway. Annulin B and other pyranonaphthoquinone derivatives that show structural similarity to DAL are recently reported to be potential IDO inhibitors (Kumar et al., 2008).

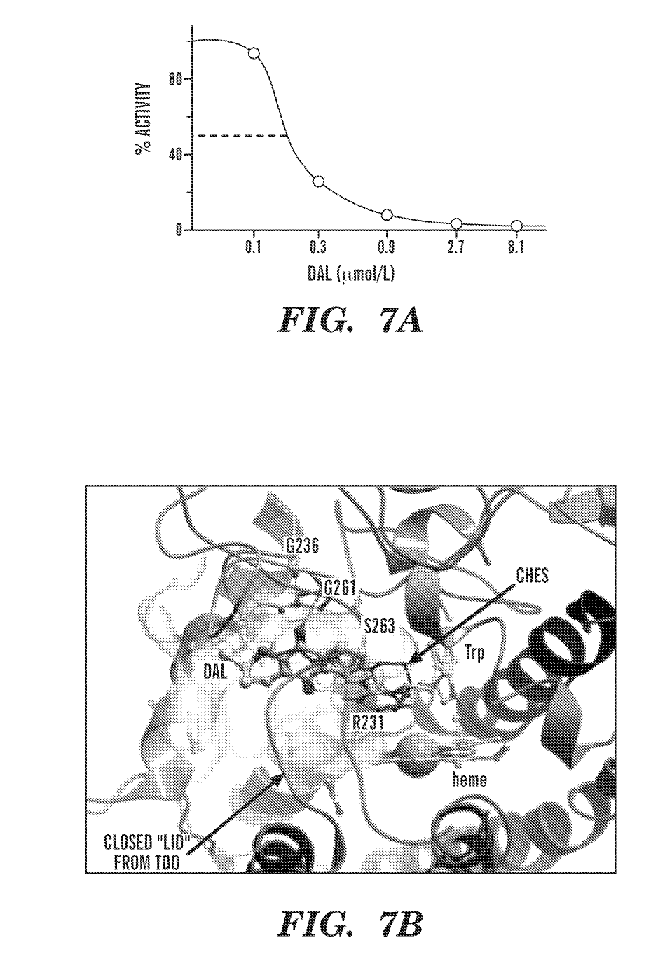

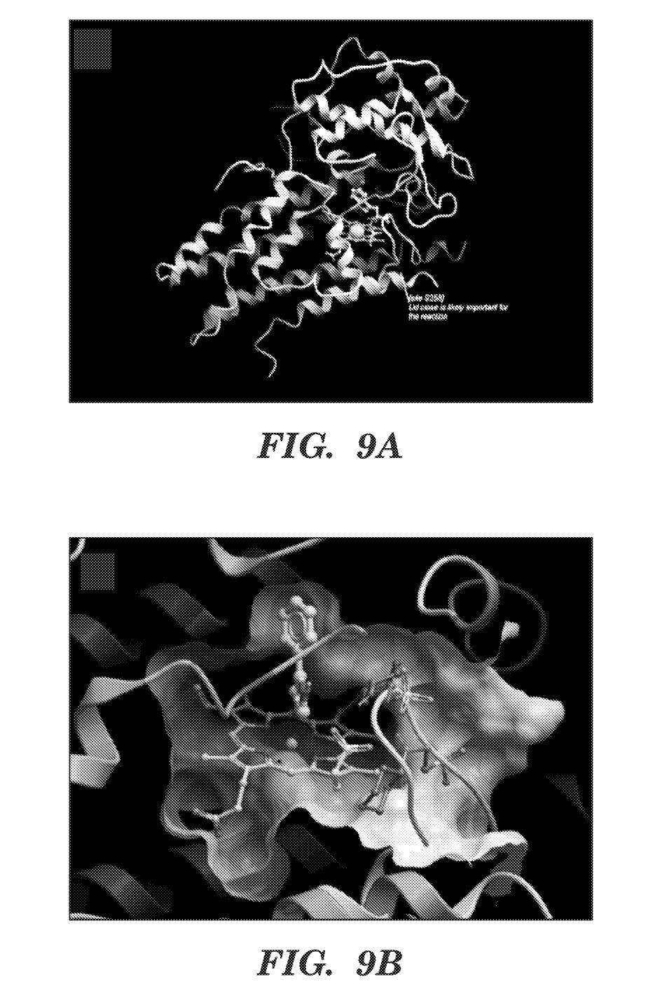

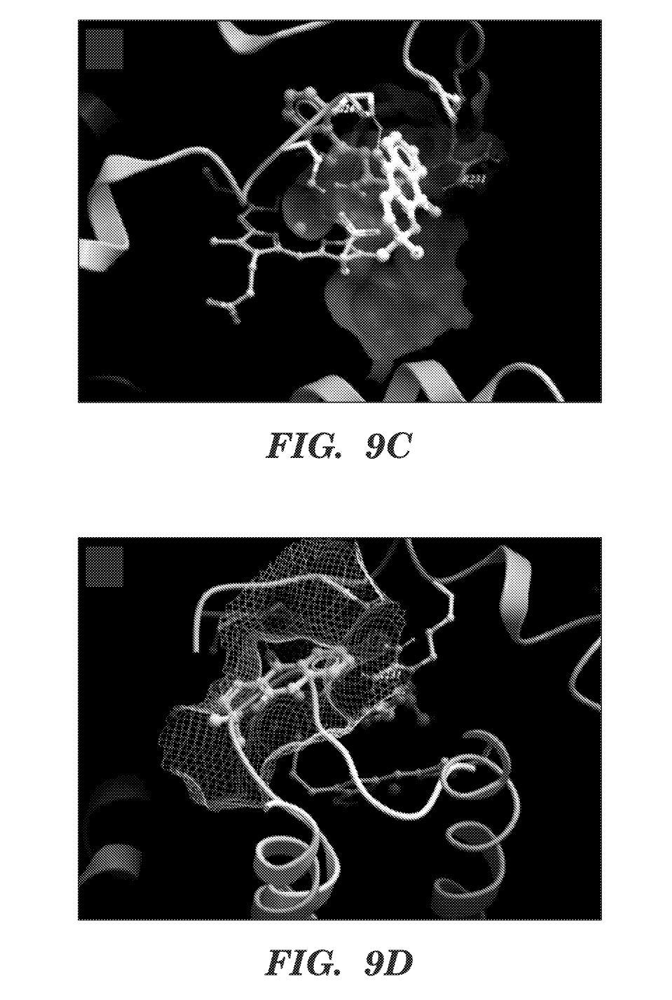

To determine whether DAL is an immuno-modulator, the effect of DAL on IDO activity was measured. To monitor IDO enzymatic activity, HEK 293 cells were engineered to express IDO and treated with DAL in a 10-dose IC₅₀ mode with threefold serial dilutions. The IDO activity was analyzed by the presence of kynurenine (the first stable catabolite downstream of IDO) with a spectrophotometric assay (Katz et al., 2008; Littlejohn et al., 2000). The results of the assay indicate that DAL significantly reduces IDO activity in cells with an IC₅₀ of about 0.3 μM (FIG. 7A). Because DAL inhibits recombinant purified IDO in the enzymatic assay, it is likely that DAL physically interacts with IDO1, forming a complex that represses IDO1 activity. To elucidate a possible structural basis for this inhibition, comparative modeling and molecular docking were used (FIGS. 9A-9D). A structure and sequence comparison of human IDO with a related bacterial enzyme, tryptophan 2,3-dioxygenase (TDO) from Xanthomonas campestris, revealed a possible critical role for the residues 361-379 in IDO. These residues are disordered in all existing IDO structures (Thackray et al., 2008); however, in the tryptophan-bound bacterial TDO structure, the corresponding loop is well-ordered and forms a “lid” on the binding pocket. Similar to human IDO1, bacterial TDO is a heme enzyme catalyzing the conversion of tryptophan into N-formyl-kynurenine. The binding site residues are strongly conserved between the two enzymes: in particular, the lid sequences share the common motif [K/R]GTGG[T/S]. It was therefore assessed that if lid closure may be a critical step for the efficient reaction catalysis by IDO. Because the inhibition of IDO by DAL is non-competitive with respect to the substrate, L-tryptophan (He et al., 2008), not wishing to be bound by theory, DAL is likely to bind concurrently with tryptophan. No structures of IDO-tryptophan complexes have been solved to date; therefore, a model of the complex was built using an inhibitor-bound structure of human IDO (Sugimoto et al., 2006) and a tryptophan-bound structure of a bacterial TDO (Thackray et al., 2008). This model was further used to build the ternary IDO/Trp/DAL complex by flexible docking. The deduced 3D model of IDO/Trp/DAL is shown in FIG. 7B, which indicates that DAL simultaneously binds to the substrate and occupies the allosteric pocket on the enzyme surface. By doing so, DAL prevents the closure of the binding site lid formed by IDO residues 361-379, which is critical for the efficient dioxygenase reaction. In combination with experimental data, the model characterizes DAL as a potent allosteric inhibitor of IDO.

Angiogenesis inhibitors targeting VEGF signaling pathway have proven to be an efficacious anticancer treatment strategy, and can be used to treat other non-malignant diseases involving abnormal vasculature or angiogenesis. Novel targets for drug discovery are still needed to diversify antiangiogenic treatment and enhance safety for the benefit of cancer and other patients. To improve the diversity of options for antivascular therapy, a high-throughput screen for small molecules that affect cell adhesion was applied. In accordance with various embodiments described herein, the non-toxic natural plant product, DAL, with antivascular properties, can be used to treat various angiogenic or vascular diseases described herein. In one embodiment, DAL with antivascular properties also provides for an antitumor effect, and thus treatment of cancer. Without wishing to be bound by theory, DAL affects cell adhesion by promoting the ubiquitination and proteasomal degradation of the Rho-GTPase Rac1, which has been noted to play a role in cell-cell adhesion stability. Further, DAL can inhibit activity of IDO, which is an immunomodulatory enzyme responsible for promoting immune tolerance, e.g., in cancer. This small molecule can serve as a lead compound for the development of a novel class of antivascular drugs with relatively low toxicity.

### Example 8

### Effects of DAL on Invasion and Metastasis in Triple-Negative Breast Cancers

Triple-negative tumors (estrogen receptor-negative, progesterone receptor-negative, and C-erbB-2-negative; ER-PR-HER2-) represent the most aggressive breast cancer subtype and have the worst prognosis despite advancements in modern therapeutics. Patients with triple-negative breast cancer often have larger tumors, are at higher risk of early disease relapse, and have reduced overall survival compared to patients with other breast cancer types. Without wishing to be bound by theory, the poor prognosis associated with triple-negative cancers arises primarily because of increased tumor invasion and metastasis. Because metastases constitute the major cause of cancer-related deaths, it is beneficial to understand the cellular and molecular events of metastatic disease. While molecular targeted therapies have significantly benefited patients with ER-positive and HER-positive metastatic breast cancer, there are currently no effective therapies to treat patients with metastatic triple-negative breast cancer.

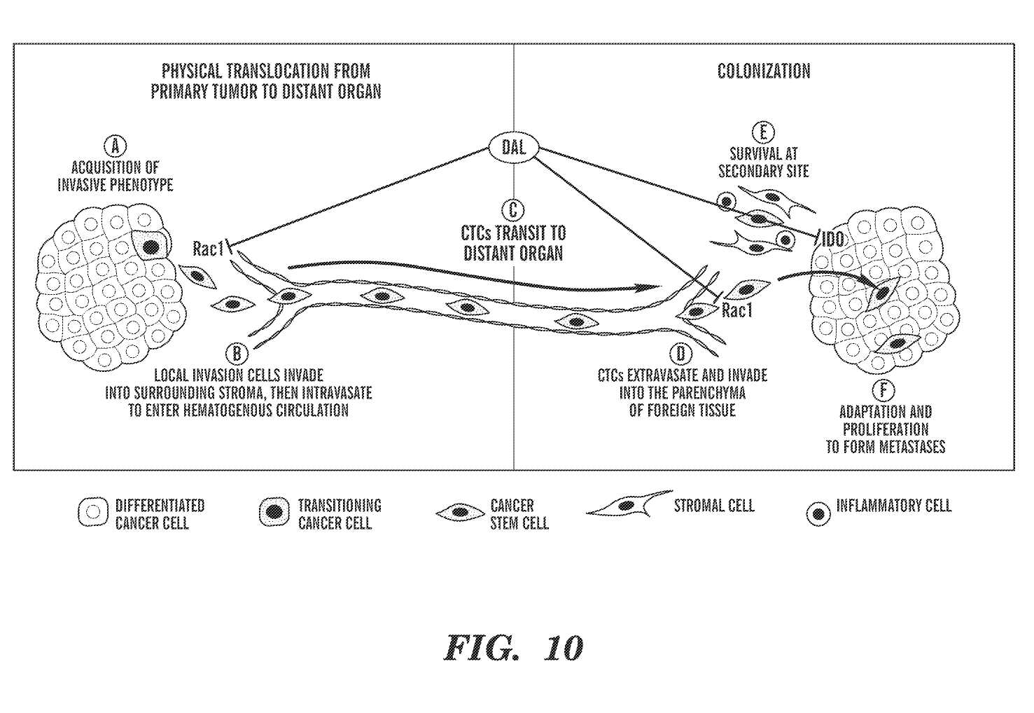

Due to the poor prognosis associated with breast cancer metastasis, there is an urgent need for agents that target this process. Without wishing to be bound by theory, in order for a cancer cell to metastasize, the cancer cell generally leaves the primary tumor, enters lymphatic or blood vessels, survives in the circulation, attaches to the endothelium of a distant organ, and subsequently forms colonies—either inside the vessel or in the target organ after extravasation (FIG. 10). After extravasation, cancer cells generally adapt and survive in a new, hostile environment. Triple-negative breast cancer cells in particular exhibit phenotypes that allow them to escape effective immune surveillance and response through immunosuppressive pathways. Invasion of the surrounding stroma and blood vessels is a critical component of this cascade, playing an important role in the initial and final steps of metastasis.

It is known that enhanced cell motility is generally correlated with increased metastatic potential in animal models and with a poor prognosis in human cancers. Cell motility generally requires dynamic remodeling of the actin cytoskeleton and involves alterations of cell adhesion molecules such as β-integrins and α-catenin that contribute to stabilization of cell attachment. Accordingly, it is sought to determine if alterations of actin cytoskeleton can be indicative of the ability of cells to attach to their substrate and therefore their metastatic potential. To this end, a unique screening strategy to identify potential agents that target cell adhesion was developed, as described in Example. Using this strategy, 50,000 compounds were screened in a high-throughput manner and 86 compounds affecting cellular adhesion and actin assembly were selected. The compounds that were nontoxic to normal cells and zebrafish were then selected. Among them, dehydro-α-lapachone (DAL), a natural product from the Tabebuia Avellanedae tree, was identified. DAL was further evaluated for toxicity in severe combined immunodeficiency (SCID) mice. It was determined that mice were tolerant to intraperitoneal doses of up to 100 mg/kg with no signs of toxicity. As shown in Example 7, DAL can also confer immunosuppressive characteristics to tumors and can interfere with cancer cell motility through remodeling of the actin cytoskeleton.

The use of DAL for treatment of triple-negative breast cancer is novel, because (1) the metastasis treatment field is virtually unexplored, as most of the anti-cancer drugs targets the primary tumor only; and (2) DAL that can target Rac1 and IDO for preventing metastasis can be readily translated into the clinic due to its low toxicity. To evaluate anti-metastatic effects of DAL in triple-negative breast cancers, it is, in part, sought to determine the effects of DAL on cancer cell motility. To this end, breast cancer cells such as 4T1-GFP and MDA-MB-231-GFP cells are used in a transwell migration assay known in the art. The migration of cells are assessed at various time points after seeding, e.g., 6 hr, 12 hr, 18 hr and 24 hr, or longer (if needed, e.g., to measure a distinct effect of DAL treatment on cell motility) after seeding. Invasion in the transwell system are performed in the absence and the presence of DAL, for example, at a concentration of 1 μM. Different DAL concentrations can be used to determine its effect on cancer cell motility based on numbers of migrated cells. The cell images are captured at the specified time points and are processed to quantify the fraction of the cells migrated to the bottom side of the membrane. Image analysis can be performed by any art-recognized software, e.g., Matlab software.

In addition to employing transwell migration assay to assess cancer cell motility, a scratch wound assay can also be used to determine breast cancer cell motility based on the healing rate or wound closure rate of a wounded monolayer. To create a “wound”, a cancer monolayer is mechanically disrupted, e.g., with a pipette tip, and the migration of cells to “heal” the wound is then monitored using microscopy. DAL treatment (e.g., at a concentration 3 μM) is compared with the control in 24 hours after wounding. Minimal or no movement of cells treated with DAL resulting in a failure of the wound closure (as compared to the control) can be indicative of DAL inhibiting or reducing breast cancer cell motility.

In order to evaluate whether DAL has an effect on the invasive behavior of cancer cells in vivo, GFP-labeled breast cancer cells (e.g., GFP-labeled 4T1 tumor cells) are implanted into the mammary fat pad of mice and are allowed to grow, e.g., for about 10 days. In one embodiment, DAL is injected daily at 37.5 mg/kg in the treated group, while the mice in the control group do not receive DAL. In some embodiments, the mice in the control group can receive saline without DAL. The mice are then sacrificed after completion of the administration protocol, and breast sections are used for assessment of distribution of green cells from tumor edges, e.g., in three independent experiments in each group. It is contemplated that DAL can inhibit cancer cell motility in in vitro and in in vivo animal models.

To evaluate anti-metastatic effects of DAL in triple-negative breast cancers, it is, in part, sought to evaluate the effects of DAL on anti-tumor immunity. To this end, DAL effects on lung metastasis from breast cancer are compared between immunodeficient and immunocompetent mice. For example, orthotopic spontaneous murine breast cancer metastasis model are used as it is clinically relevant and includes all the steps of the metastasis process. To compare the effects of DAL on metastasis between immunodeficient and immunocompetent mice, highly metastatic breast cancer cells (e.g., 4T1 cells) constitutively expressing GFP are injected into mice. Female BALB/c and SCID mice are injected with 1×10⁵ 4T1 cells in the mammary fat pad. For this human tumor xenograft study, immunodeficient mice (SCID mice, T and B cell deficient) are used. Without wishing to be bound by theory, the lack of T and B-lymphocytes can have some indirect effects on cancer-host interaction and interfere with DAL effects. As such, murine E0771 breast cancer cells grown orthotopically in syngeneic immunocompetent mice (C57BL/6 mice) are also evaluated.

Primary 4T1 tumors (and E0771 tumors) are removed from the mice on day 5 (or when the tumor size reaches about 6 mm in diameter). After removal of the primary tumors, the animals are treated with DAL for about 20 days. In one embodiment, DAL (e.g., at a concentration of about 37.5 mg/kg) is administered daily; this administration schedule is based on the medium plasma half-life (1.7 hours) of this compound after intraperitoneal dosing. In this particular embodiment, DAL treatment is initiated after removal of a primary tumor to be more clinically relevant. Although, in such embodiment, DAL is not administered early enough to block the intravasation step of metastasis, this administration protocol mimics a situation most frequently seen in clinical settings (i.e., start of drug treatment after removal of the primary tumor). However, without wishing to be limited, DAL treatment can also be initiated right after implantation of tumor cells to the mammary pad.

One group of animals is sacrificed after completion of DAL treatment on day 25. Metastasis can be categorized as micrometastases, or macrometastases (<2 mm, or >2 mm in diameter, respectively). Visible lung macrometastases on the lung surface are counted, e.g., using a stereomicroscope. Each lung is then fixed in paraformaldehyde/lysine/periodate solution, paraffin-embedded, sectioned (˜5 μm thick) and stained with hematoxylin and eosin (H&E) to visualize micrometastases. The micrometastases are counted, for example, in five randomly selected sections, from each lung. It is contemplated that DAL can further reduce metastasis in immunocompetent mice, at least partly, on account of its immunomodulatory properties.

DAL generally regulates activities of both Rac1 and indoleamine-2,3-dioxygenase (IDO). To specifically evaluate the anti-metastatic effects of DAL-induced IDO repression, micrometastases and macrometastases are measured in female BALB/c mice bearing primary orthotopic 4T1 (triple-negative) and shRac1-4T1 (triple-negative infected with shRac1 adenoviruses) mammary tumors in mammary fat pads. Primary tumors are removed, e.g., when they reach a size of about 6 mm in diameter. After removal of the primary tumors, the animals receive intraperitoneal injections of DAL for about 20 days. Analysis of the micrometastases and macrometastases are performed as described above. In some embodiments, rather than using shRNA for Rac1 alone to significantly reduce Rac1 expression in different breast cancer cell lines, a commercially-available blocking peptide (W56) that blocks Rac1 function can be used instead or in combination with the Rac1 shRNA. The blocking peptide (W56) comprises residues 45-60 of the guanine nucleotide exchange factor (GEF) recognition/activation site of Rac1 and selectively inhibits Rac1 interaction with Rac1-specific GEFs TrioN, GEF-H1 and Tiam1. Without wishing to be bound by theory, it is contemplated that when DAL can repress the Rho-GTPase Rac1 and decrease cancer cell motility, DAL can have less anti-metastatic effects in shRac1-4T1 tumors compared to 4T1 tumors.

Since DAL has a potential to inhibit certain pathways in the metastatic cascade (e.g., cancer cell invasion and immune evasion), it is next sought to determine whether DAL treatment can translate to inhibition of metastases in vivo. Accordingly, orthotopic murine (4T1) and human (MDA-MB-231) spontaneous triple-negative breast cancer metastasis mouse models are used. These are clinically relevant tumors that recapitulate all steps of the metastasis cascade. Tumor cells are injected in the mouse mammary fat pad (4T1 cells in female BALB/c mice; MDA-MB-231 cells in female SCID mice), and the primary tumors are removed once tumor size reaches a size of about 6 mm in diameter. Mice are then treated with DAL, e.g., at a concentration of about 37.5 mg/kg (or vehicle) daily for about 20 days via intraperitoneal injections. Metastasis are categorized as micrometastases, or macrometastases (<2 mm, or >2 mm in diameter, respectively). Visible lung macrometastases on the lung surface are counted with a stereomicroscope. Each lung is then fixed in paraformaldehyde/lysine/periodate solution, paraffin-embedded, sectioned (˜5 μm thick) and stained with H&E to visualize micrometastases. The micrometastases are counted, e.g., in five randomly selected sections, from each lung. In parallel, another group of animals is used to determine the effect of DAL on mouse survival (e.g., Kaplan-Meier survival assay).

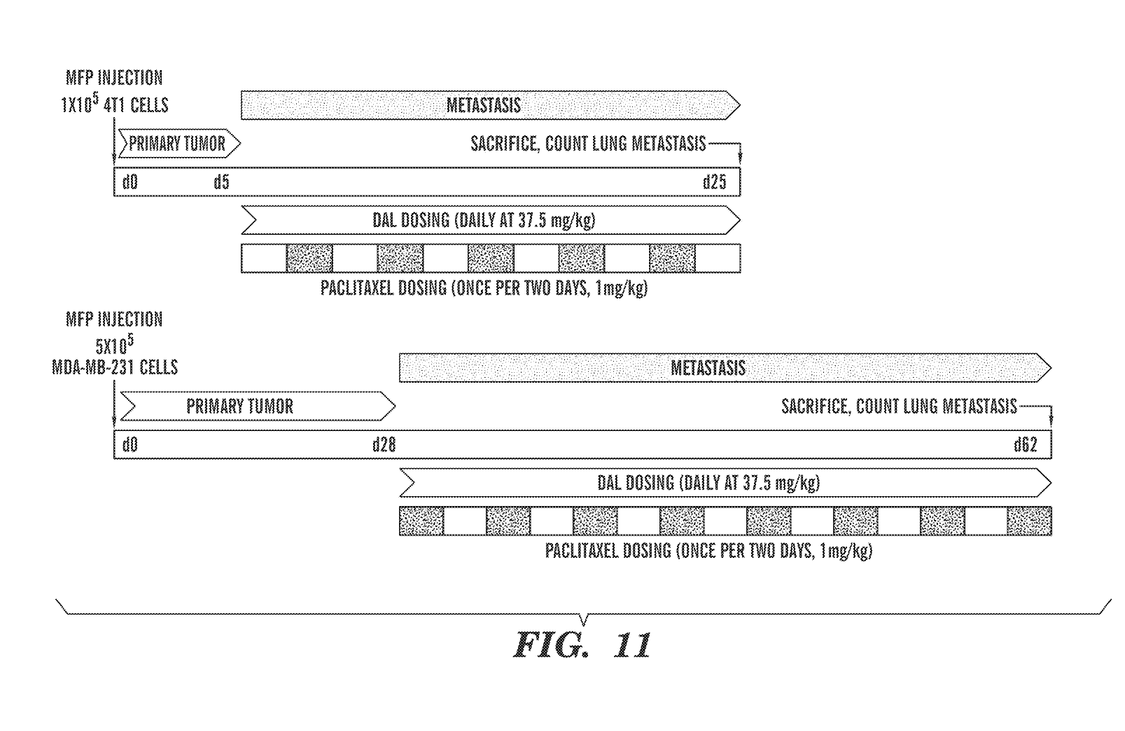

In the clinic successful targeted therapy is often combined with standard chemotherapy in order to achieve maximal and even synergistic effects. Since paclitaxel is a standard treatment for triple-negative tumors, the compound DAL in combination with paclitaxel is also evaluated for its anti-metastatic efficacy for treatment of metastasis. To assess the effects of combining paclitaxel and DAL, 4T1 and MDA-MB-231 breast cancer metastasis models are used. After removal of the primary tumors, the animals are treated with DAL compound (or vehicle) for about 20 days (for 4T1 model) or about 34 days (for MDA-MB-231 model). Tumor bearing mice receive intraperitoneal injections of a combination treatment of DAL and paclitaxel, or paclitaxel alone. For example, in one embodiment, DAL is injected daily in a dose of about 37.5 mg/kg and paclitaxel is injected once every two days in a dose of about 1 mg/kg, as shown in FIG. 11. The effects of combined therapy are evaluated by counting micrometastases and macrometastases. In parallel, additional groups of animals are used to determine the effect of a combination treatment of paclitaxel and DAL on mice survival (e.g., Kaplan-Meier survival assay). It is contemplated that a combined therapy with DAL and paclitaxel can have synergistic effects on repression of metastases.

Different modalities to monitor and track metastasis (e.g., without limitations, whole body imaging by measuring luc/Gluc activity in cancer metastatic cells and systemic blood glucose measurements, and/or intravital microscopy measurements) can also be used, e.g., to establish the optimum time for metastasis development for each murine and human model.

In some embodiments, the growth of the cell lines in vivo prior to and after genetic manipulations can vary slightly. As such, the protocols described herein can be optimized at the time of measuring the primary tumor growth and occurrence of metastasis. For example, if tumor growth is delayed in an animal model, the observation time can be extended to allow detection of metastasis.

### REFERENCES

1. Carmeliet P & Jain RK. Molecular mechanisms and clinical applications and angiogenesis (2011). Nature 473: 1-10.
2. Folkman J (2007) Angiogenesis: an organizing principle for drug discovery? Nat Rev Drug Discov 6(4):273-286.
3. Chung A S, Lee J, & Ferrara N (2010) Targeting the tumour vasculature: insights from physiological angiogenesis. Nature reviews 10(7):505-514.
4. Nagy J A, Chang S H, Dvorak A M, & Dvorak H F (2009) Why are tumour blood vessels abnormal and why is it important to know? Br J Cancer 100(6):865-869.
5. Olsson A K, Dimberg A, Kreuger J, & Claesson-Welsh L (2006) VEGF receptor signalling—in control of vascular function. Nat Rev Mol Cell Biol 7(5):359-371.
6. Bergers G & Hanahan D (2008) Modes of resistance to anti-angiogenic therapy. Nature reviews 8(8):592-603.
7. Jain R K, et al. (2009) Biomarkers of response and resistance to antiangiogenic therapy. Nat Rev Clin Oncol 6(6):327-338.
8. Carmeliet P, et al. (1999) Targeted deficiency or cytosolic truncation of the VE-cadherin gene in mice impairs VEGF-mediated endothelial survival and angiogenesis. Cell 98(2):147-157.
9. Corada M, et al. (1999) Vascular endothelial-cadherin is an important determinant of microvascular integrity in vivo. Proceedings of the National Academy of Sciences of the United States of America 96(17):9815-9820.
10. Jain R K (2005) Normalization of tumor vasculature: an emerging concept in antiangiogenic therapy. Science 307(5706):58-62.
11. Dejana E (2004) Endothelial cell-cell junctions: happy together. Nat Rev Mol Cell Biol 5(4):261-270.
12. Hashizume H, et al. (2000) Openings between defective endothelial cells explain tumor vessel leakiness. Am J Pathol 156(4):1363-1380.
13. Hobbs S K, et al. (1998) Regulation of transport pathways in tumor vessels: role of tumor type and microenvironment. Proceedings of the National Academy of Sciences of the United States of America 95(8):4607-4612.
14. Corada M, et al. (2002) A monoclonal antibody to vascular endothelial-cadherin inhibits tumor angiogenesis without side effects on endothelial permeability. Blood 100(3):905-911.
15. Gumbiner B M (2005) Regulation of cadherin-mediated adhesion in morphogenesis. Nat Rev Mol Cell Biol 6(8):622-634.
16. Borisy G G & Svitkina T M (2000) Actin machinery: pushing the envelope. Curr Opin Cell Biol 12(1):104-112.
17. Li C J, Averboukh L, & Pardee A B (1993) beta-Lapachone, a novel DNA topoisomerase I inhibitor with a mode of action different from camptothecin. J Biol Chem 268(30):22463-22468.
18. Planchon S M, et al. (1995) Beta-lapachone-mediated apoptosis in human promyelocytic leukemia (HL-60) and human prostate cancer cells: a p53-independent response. Cancer research 55(17):3706-3711.
19. Huang C C, Lawson N D, Weinstein B M, & Johnson S L (2003) reg6 is required for branching morphogenesis during blood vessel regeneration in zebrafish caudal fins. Dev Biol 264(1):263-274.
20. Whitehead G G, Makino S, Lien C L, & Keating M T (2005) fgf20 is essential for initiating zebrafish fin regeneration. Science 310(5756):1957-1960.
21. Nechiporuk A & Keating M T (2002) A proliferation gradient between proximal and msxb-expressing distal blastema directs zebrafish fin regeneration. Development 129(11):2607-2617.
22. Brown E, et al. (2003) Dynamic imaging of collagen and its modulation in tumors in vivo using second-harmonic generation. Nat Med 9(6):796-800.
23. Garkavtsev I, et al. (2004) The candidate tumour suppressor protein ING4 regulates brain tumour growth and angiogenesis. Nature 428(6980):328-332.
24. Vakoc B J, et al. (2009) Three-dimensional microscopy of the tumor microenvironment in vivo using optical frequency domain imaging. Nat Med 15(10):1219-1223.
25. Anderson J M & Van Itallie C M (2008) Tight junctions. Curr Biol 18(20):R941-943.
26. Miyoshi J & Takai Y (2005) Molecular perspective on tight-junction assembly and epithelial polarity. Adv Drug Deliv Rev 57(6):815-855.
27. Tzima E (2006) Role of small GTPases in endothelial cytoskeletal dynamics and the shear stress response. Circ Res 98(2):176-185.
28. Tsukita S, Furuse M, & Itoh M (2001) Multifunctional strands in tight junctions. Nat Rev Mol Cell Biol 2(4):285-293.
29. Ridley A J (2006) Rho GTPases and actin dynamics in membrane protrusions and vesicle trafficking. Trends Cell Biol 16(10):522-529.
30. Wojciak-Stothard B & Ridley A J (2002) Rho GTPases and the regulation of endothelial permeability. Vascul Pharmacol 39(4-5):187-199.
31. Reinstein E & Ciechanover A (2006) Narrative review: protein degradation and human diseases: the ubiquitin connection. Ann Intern Med 145(9):676-684.
32. Bence N F, Sampat R M, & Kopito R R (2001) Impairment of the ubiquitin-proteasome system by protein aggregation. Science 292(5521):1552-1555.
33. Wong H K, et al. (2008) Blocking acid-sensing ion channel 1 alleviates Huntington's disease pathology via an ubiquitin-proteasome system-dependent mechanism. Hum Mol Genet. 17(20):3223-3235.
34. He K L, et al. (2008) Endothelial cell annexin A2 regulates polyubiquitination and degradation of its binding partner S100A10/p11. J Biol Chem 283(28):19192-19200.
35. Jain R K, Munn L L, & Fukumura D (2011) Transparent Window Models and Intravital Microscopy: Imaging Gene Expression, Physiological Function and Therapeutic Effects in Tumors. Tumor Models in Cancer Research, ed Teicher BA (Humana Press, New York, N.Y.), pp 641-681.

The references cited herein and throughout the application are incorporated herein by reference in their entirety. Although the foregoing invention has been described in some detail by way of illustration and example for the purposes of clarity of understanding, one skilled in the art will easily ascertain that certain changes and modifications may be practiced without departing from the spirit and scope of the appended claims. All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating or preventing a condition involving abnormal vasculatures in a subject, the method comprising administering to the subject a composition comprising a compound of formula (I) or an analog, derivative, isomer, prodrug, or pharmaceutically acceptable salt thereof, thereby reducing angiogenesis or existing blood vessels in or surrounding a target tissue, wherein the compound of formula (I) has a structure: 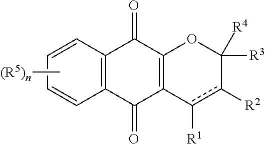
wherein:
R¹ and R² are each independently selected from hydrogen, halogen, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)O R⁶, C(O)R⁶, C(O)OR⁶, SR⁶, or SO₂R⁶, each of which can be optionally substituted;
R³ and R⁴ are each independently selected from hydrogen, alkyl, alkenyl, cyclyl, heterocyclyl, aryl, heteroaryl, each of which can be optionally substituted;
R⁵ is each independently selected from halogen, CN, CF₃, alkyl, alkenyl, cyclyl, heterocyclyl, aryl, heteroaryl, NO₂, OR⁶, OC(O)R⁶, OC(O)OR⁶, N(R⁶)₂, NHC(O)R⁶, NHC(O)OR⁶, C(O)R⁶, C(O)OR⁶, SR⁶, or SO₂R⁶, each of which can be optionally substituted;
R⁶ is independently selected for each occurrence, hydrogen, alkyl, alkenyl, alkynyl, cyclyl, heterocyclyl, aryl, or heteroaryl, each of which can be optionally substituted;
the dotted line between the carbons to which R¹ and R² are bonded represents an optional double bond; and
n is 0, 1, 2, 3, or 4.

2-78. (canceled)

79. The method of claim 1, wherein the composition is administered in pulses.

80. The method of claim 1, wherein the composition is administered via sustained release.

81. The method of claim 80, wherein the composition comprises a delivery system adapted for sustained release of the compound of formula (I).

82. The method of claim 81, wherein the delivery system is selected from the group consisting of polymer-based systems, lipid-based systems, hydrogel release systems, liposome-based systems, phospholipid-based systems, silastic systems, peptide-based systems, implants, stents, transdermal patches, and any combinations thereof.

83. The method of claim 1, wherein the compound of formula (I) is conjugated to a macromolecule.

84. The method of claim 1, wherein the composition is administered to the subject over a period of at least about 2 months.

85. The method of claim 1, wherein the compound of formula (I) is dehydro-alpha-lapachone or an analog, derivative, isomer, prodrug, or pharmaceutically acceptable salt thereof.

86. The method of claim 85, wherein the compound of formula (I) is derived from Tabebuia Avellaneda tree.

87. The method of claim 1, wherein the compound of formula (I) is administered in an effective amount of about 0.001 mg/kg to about 500 mg/kg.

88. The method of claim 1, wherein the subject is determined to have acquired or have a risk for acquiring resistance to at least one VEGF inhibitor.

89. The method of claim 1, wherein the condition involving abnormal vasculatures is a triple-negative breast cancer.

90. The method of claim 1, wherein the condition involving abnormal vasculatures is selected from the group consisting of tuberculosis, autoimmune diseases, wound repair, hypertrophic scar or keloid, neointimal hyperplasia, psoriasis, macular degeneration, age-related macular degeneration (AMD), thyroid hyperplasia, preeclampsia, hemangiomas, rheumatoid arthritis and osteoarthritis, Alzheimer's disease, obesity, pleural effusion, atherosclerosis, endometriosis, diabetic retinopathies, other retinopathies, ocular neovascularizations, edemas, chronic obstructive pulmonary diseases, asthma, cystic fibrosis, transplant rejection, allergic reaction, multiple sclerosis, epithelial infection, and conditions involving or characterized by vascular hyperpermeability, inflammation, angiogenesis or vascular hyperproliferation, but excluding cancer and metastatic tumors.

91. The method of claim 1, wherein the condition involving abnormal vasculatures is an ocular disease or disorder.

92. The method of claim 91, wherein the ocular disease or disorder is selected from the group consisting of age-related macular degeneration (AMD), proliferative diabetic retinopathy (PDR), diabetic macular edema (DME), neovascular glaucoma, corneal neovascularization (trachoma), and pterygium.

93. The method of claim 1, wherein the compound of formula (I) is administered in a combination therapy.

94. The method of claim 93, wherein the combination therapy comprises administering to the subject at least one active agent selected from the group consisting of therapeutic agents, anti-angiogenic or anti-vascular agents, anti-inflammation agents, VEGF inhibitors, antibiotics, anti-viral agents, anti-fungal agents, anti-protozoal agents, hormones, radioactive agents, toxins, anesthetics, and any combinations thereof.

95. The method of claim 1, wherein the compound of formula (I) decreases cellular adhesion.

96. The method of claim 1, wherein the compound of formula (I) inhibits or reduces activity of an immunomodulatory enzyme, thereby increasing immune activity in the subject.

97. The method of claim 96, wherein the immunomodulatory enzyme is indoleamine-pyrrole 2,3-dioxygenase (IDO).
